SEQUENCE LISTING (15 sequences)

SEQ ID NO: 1 - protein, 259 aa, PRD1 bacteriophage
MAKKKPVEKNGLVYKEFQKQVSNLKKAGLIPKTLDVRKVKPTKHYKGLVSKYKDVATGGAKLAAIPNPAVIETLEARGESIIKKGGKAYLKARQQINQRGQIVNPFTVRVTKRGEVVRRYRKTTPEGKPVYITQRELPIKFENMEQWLTELKAAGFQLQPGEQIYFTFNGNYSRRTYTSFDEAFNKFMTYDIIIDAVAGKLKVEDEADLVKSVGFQRISGPEAKAYNRNRIVLPEMQFSQAAKKKYKRRQKRGYGSKGV

SEQ ID NO: 2 - protein, 553 aa, PRD1 bacteriophage
MPRRSRKKVEYKIAAFDFETDPFKHDRIPKPFSWGFYNGEIYKDYWGDDCIEQFIYWLDTIEEPHVIYAHNGGKFDFLFLMKYFRGKLKIVNGRILEVEHGIHKFRDSYAILPVPLAASDEKIEIDYGKMERETREQHKAEILEYLKGDCVTLHKMVSLFIAEFGMRLTIGGTAMNELKQFHPYDPVRKGFDEAMRPFYFGGRCQAFEKGIIEDDIKVYDVNSMYPHAMRNFRHPFSDEFYEANEITEETYFIEWEGENNGAVPVRTKTGLDFNQRSGIFHTSIHEWRAGIDTGTIKPNRIIRTINFTETTTFGAFIDHFFSKRDAAKKAGDLFHNIFYKLILNSSYGKFAQNPENYKEWCITEGGIYLEGYDGEGCEVQEHLDYILWGRPAEMFNYFNVAVAASITGAARSVLLRALAQAERPLYCDTDSIICRDLKNVPLDAYQLGAWDLEATGDKIAIAGKKLYALYAGDNCVKIASKGASLVPRDIGFLMPPDMEPKAAKKVAQQKAKNIGGEKILKVANGGVYDFVNDAPSFKLNGNVQFIKRTIKGT

SEQ ID NO: 3 - protein, 160 aa, PRD1 bacteriophage
MEIVSKLTLKTIGAQPKPHSVKENTALASIYGRVRGKKVGQSTFGDFIKFEGEFEGVNIATGEVFRSGALILPKVLESLLAGAVDGENTVDFAVEIWAKPSEKGNTGYEYGVKPLIEPAASDELAALRNQVKAALPAPAAAGEAAAEAKPAAKAKAKAEA

SEQ ID NO: 4 - protein, 94 aa, PRD1 bacteriophage
MEKQTENTRPECPKAFYFVSIPGDFGQTPFASSLMYGSTALAAVYQVKGAIRVVSEHFDLRFADNGFTPAGVTQAEWLGKLITETFGFRLELFL

SEQ ID NO: 5 - DNA, 3536 bp, synthetic construct
  source 1..3536: mol_type = other DNA
taccccaaattgcgcccctgcaatttgctgacggtttaacggaaggcggaaacatggcgaagaaaaaaccagtagaaaaaaatgggcttgtttataaagagtttcaaaaacaagtttcaaatttgaagaaagccggactaatccctaaaacccttgacgtgcgaaaagtcaagccaacaaaacactataaaggattggtaagcaaatataaagacgttgcaacagggggcgctaaacttgcagcaatccctaaccccgccgttattgaaacgcttgaagcgcggggcgaatccatcattaagaaaggcggcaaggcgtatctgaaagcccgccagcaaataaaccagcgcgggcaaattgtaaacccctttacggttcgcgtaaccaaacgcggcgaagtggtgcgccgctaccgcaagactaccccggaaggcaagcccgtttatatcacgcaacgggaattgcctattaagtttgaaaatatggaacagtggcttactgaattaaaggccgctggttttcaattgcaaccgggcgaacaaatctatttcacttttaacggcaactattcccgccgtacctatacgtcatttgatgaagcgttcaataaatttatgacgtatgacattattattgatgcggtggccggaaaattaaaagtagaagatgaagccgatttagttaagtcggtaggctttcaacgtatcagcggccccgaagccaaggcgtataaccgtaaccgtattgtattgcctgaaatgcaatttagccaagcggctaaaaagaaatacaagcgccgtcaaaaacgcggctatggcagcaagggggtttaagatatgccgcgccgttcccgtaaaaaggtggaatataaaattgccgcctttgactttgaaactgaccctttcaagcatgaccgaatccctaaaccgttttcatggggtttttataatggcgaaatttataaagactattggggcgatgattgcatagaacagtttatttactggctggataccatagaagaaccgcacgttatatacgctcataacggcggcaagtttgattttctttttctcatgaaatactttcgcgggaaattgaaaatagttaatgggcgtattttggaagtagaacacggcatccataaattccgcgatagttatgcaatcctgccggtgccgcttgctgccagcgatgaaaagatagaaattgattatggcaagatggaaagggaaacacgcgaacagcacaaggcggaaattttagaatacctgaaaggcgattgtgtaaccctgcataaaatggtttctttatttattgctgaatttggaatgcgcctaaccataggcggtacggcaatgaatgaattaaaacagttccacccttatgaccctgtgcgcaaaggctttgatgaagccatgcgccccttttattttggcggaaggtgccaagcattcgagaaaggaataattgaagatgatataaaagtttatgatgttaatagtatgtacccccatgctatgcgaaatttccgccatcctttcagcgatgaattttatgaagccaatgaaataacagaagaaacttattttattgaatgggaaggcgagaataacggcgcggtgcctgttaggactaaaacaggtttagactttaatcagcgtagcggcattttccatacgtcaatccatgaatggcgggcgggtattgataccggcacgattaaacctaatcggattataaggacaatcaattttactgaaacaaccactttcggcgcattcattgaccatttctttagcaagcgtgacgctgccaaaaaggcgggtgatttattccacaatattttttacaaactgattttaaatagcagttatgggaagtttgcacaaaaccccgaaaattataaagagtggtgcataacggaaggcggcatttatttagaaggctatgacggcgaagggtgcgaagtacaggaacatttagactatattttatggggtaggcccgctgaaatgtttaattattttaacgtggcagtggcggcaagtattacaggcgcggcccgttccgttttattgcgagcattggcgcaagcggaaaggccgctttattgcgacactgattctattatctgccgtgatttaaaaaatgttccgcttgacgcataccagctaggcgcgtgggatttggaagcaaccggcgataaaatagcgattgccggtaaaaaattatatgcgctttacgctggtgataattgcgttaaaattgcaagtaagggggctagtctggttccgcgtgatattgggtttttaatgcccccggatatggaaccgaaagccgccaaaaaggtagcgcaacaaaaggctaaaaatattggtggcgagaaaattttaaaggtggctaatggcggcgtgtatgattttgtaaatgatgccccgtcatttaagctaaatggcaacgtgcaatttatcaagcgcacaatcaaaggaacataaatcgaaagggcaaacacaaaacccccgccgataacttccacttaactttaaaggtaactatcatggaaatcgtaagcaagctgactctgaaaaccattggcgcacaaccgaagccgcatagcgtaaaagaaaataccgcgctggcttccatctatggccgcgttcgcggtaagaaagttggtcaatccacctttggcgacttcatcaagtttgaaggtgaatttgaaggcgtgaatatcgccactggtgaagtgttccgttccggtgcgctgattctgccgaaggtactggaaagcctgcttgccggtgccgtggatggtgaaaacacggttgattttgcggttgaaatttgggccaagccttccgaaaagggcaacactggttatgaatatggtgtcaagccgctgattgaacccgccgcatcggatgaactggccgcgcttcgcaatcaggttaaggccgcgctgcctgcccctgccgctgccggtgaagccgctgccgaagccaagcccgccgccaaggccaaggccaaggccgaagcctaaacagcgcaccacggccccggctgataccagcgcggggccattctttacccttcaaattcaaaggtaacaaacatggaaaagcaaaccgaaaatacccgccctgaatgcccgaaggcattttattttgtatccattcccggcgactttgggcaaaccccttttgcttcatcgcttatgtatggtagcaccgcgcttgccgctgtttatcaagtcaagggcgctatccgcgtagtaagcgaacattttgatttgcggtttgctgataacggttttacgcctgctggcgttacgcaagctgaatggcttggcaagctgattactgaaactttcggctttcgcttggaactgtttctttaaaccagttttttatttcttgaatttgaaggccgcatgattcccttgcggccttttcttttgcgtatgcttccaccaaatcccgccacgttataaacggataaacaggcgcggcgcattcagttagcaggcttgacgg

SEQ ID NO: 6 - DNA, 4796 bp, synthetic construct
  source 1..4796: mol_type = other DNA
aaacgactgtcctggccgtaaccgacccagcgcccgttgcaccacagatgaaacgccgagttaacgccatcaaaaataattcgcgtctggccttcctgtagccagctttcatcaacattaaatgtgagcgagtaacaacccgtcggattctccgtgggaacaaacggcggattgaccgtaatgggataggtcacgttggtgtagatgggcgcatcgtaaccgtgcatctgccagtttgaggggacgacgacagtatcggcctcaggaagatcgcactccagccagctttccggcaccgcttctggtgccggaaaccaggcaaagcgccattcgccattcaggctgcgcaactgttgggaagggcgatcggtgcgggccacgacggccagtgaatccgtaatcatggccgacatcataacggttctggcaaatattctgaaatgagctgttgacaattaatcatcggctcgtataatgtgtggaattgtgagcggataacaatttaccccaaattgcgcccctgcaatttgctgacggtttaacggaaggcggaaacatggcgaagaaaaaaccagtagaaaaaaatgggcttgtttataaagagtttcaaaaacaagtttcaaatttgaagaaagccggactaatccctaaaacccttgacgtgcgaaaagtcaagccaacaaaacactataaaggattggtaagcaaatataaagacgttgcaacagggggcgctaaacttgcagcaatccctaaccccgccgttattgaaacgcttgaagcgcggggcgaatccatcattaagaaaggcggcaaggcgtatctgaaagcccgccagcaaataaaccagcgcgggcaaattgtaaacccctttacggttcgcgtaaccaaacgcggcgaagtggtgcgccgctaccgcaagactaccccggaaggcaagcccgtttatatcacgcaacgggaattgcctattaagtttgaaaatatggaacagtggcttactgaattaaaggccgctggttttcaattgcaaccgggcgaacaaatctatttcacttttaacggcaactattcccgccgtacctatacgtcatttgatgaagcgttcaataaatttatgacgtatgacattattattgatgcggtggccggaaaattaaaagtagaagatgaagccgatttagttaagtcggtaggctttcaacgtatcagcggccccgaagccaaggcgtataaccgtaaccgtattgtattgcctgaaatgcaatttagccaagcggctaaaaagaaatacaagcgccgtcaaaaacgcggctatggcagcaagggggtttaagatatgccgcgccgttcccgtaaaaaggtggaatataaaattgccgcctttgactttgaaactgaccctttcaagcatgaccgaatccctaaaccgttttcatggggtttttataatggcgaaatttataaagactattggggcgatgattgcatagaacagtttatttactggctggataccatagaagaaccgcacgttatatacgctcataacggcggcaagtttgattttctttttctcatgaaatactttcgcgggaaattgaaaatagttaatgggcgtattttggaagtagaacacggcatccataaattccgcgatagttatgcaatcctgccggtgccgcttgctgccagcgatgaaaagatagaaattgattatggcaagatggaaagggaaacacgcgaacagcacaaggcggaaattttagaatacctgaaaggcgattgtgtaaccctgcataaaatggtttctttatttattgctgaatttggaatgcgcctaaccataggcggtacggcaatgaatgaattaaaacagttccacccttatgaccctgtgcgcaaaggctttgatgaagccatgcgccccttttattttggcggaaggtgccaagcattcgagaaaggaataattgaagatgatataaaagtttatgatgttaatagtatgtacccccatgctatgcgaaatttccgccatcctttcagcgatgaattttatgaagccaatgaaataacagaagaaacttattttattgaatgggaaggcgagaataacggcgcggtgcctgttaggactaaaacaggtttagactttaatcagcgtagcggcattttccatacgtcaatccatgaatggcgggcgggtattgataccggcacgattaaacctaatcggattataaggacaatcaattttactgaaacaaccactttcggcgcattcattgaccatttctttagcaagcgtgacgctgccaaaaaggcgggtgatttattccacaatattttttacaaactgattttaaatagcagttatgggaagtttgcacaaaaccccgaaaattataaagagtggtgcataacggaaggcggcatttatttagaaggctatgacggcgaagggtgcgaagtacaggaacatttagactatattttatggggtaggcccgctgaaatgtttaattattttaacgtggcagtggcggcaagtattacaggcgcggcccgttccgttttattgcgagcattggcgcaagcggaaaggccgctttattgcgacactgattctattatctgccgtgatttaaaaaatgttccgcttgacgcataccagctaggcgcgtgggatttggaagcaaccggcgataaaatagcgattgccggtaaaaaattatatgcgctttacgctggtgataattgcgttaaaattgcaagtaagggggctagtctggttccgcgtgatattgggtttttaatgcccccggatatggaaccgaaagccgccaaaaaggtagcgcaacaaaaggctaaaaatattggtggcgagaaaattttaaaggtggctaatggcggcgtgtatgattttgtaaatgatgccccgtcatttaagctaaatggcaacgtgcaatttatcaagcgcacaatcaaaggaacataaatcgaaagggcaaacacaaaacccccgccgataacttccacttaactttaaaggtaactatcatggaaatcgtaagcaagctgactctgaaaaccattggcgcacaaccgaagccgcatagcgtaaaagaaaataccgcgctggcttccatctatggccgcgttcgcggtaagaaagttggtcaatccacctttggcgacttcatcaagtttgaaggtgaatttgaaggcgtgaatatcgccactggtgaagtgttccgttccggtgcgctgattctgccgaaggtactggaaagcctgcttgccggtgccgtggatggtgaaaacacggttgattttgcggttgaaatttgggccaagccttccgaaaagggcaacactggttatgaatatggtgtcaagccgctgattgaacccgccgcatcggatgaactggccgcgcttcgcaatcaggttaaggccgcgctgcctgcccctgccgctgccggtgaagccgctgccgaagccaagcccgccgccaaggccaaggccaaggccgaagcctaaacagcgcaccacggccccggctgataccagcgcggggccattctttacccttcaaattcaaaggtaacaaacatggaaaagcaaaccgaaaatacccgccctgaatgcccgaaggcattttattttgtatccattcccggcgactttgggcaaaccccttttgcttcatcgcttatgtatggtagcaccgcgcttgccgctgtttatcaagtcaagggcgctatccgcgtagtaagcgaacattttgatttgcggtttgctgataacggttttacgcctgctggcgttacgcaagctgaatggcttggcaagctgattactgaaactttcggctttcgcttggaactgtttctttaaaccagttttttatttcttgaatttgaaggccgcatgattcccttgcggccttttcttttgcgtatgcttccaccaaatcccgccacgttataaacggataaacaggcgcggcgcattcagttagcaggcttgacgggggagaccagaaacaaaaaaacacccgttagggtgttttttcgaaaaggctggtcattgtccactctctaaacgagatacttgacgagcgagttgcatcagagaatctgccaatgcacgaggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcg

SEQ ID NO: 7 - DNA, 131 bp, PRD1 bacteriophage
  source 1..131: mol_type = genomic DNA
ggggatacgtgcccctccccacctacccgcgcccctaacatttttatttccgtctgtcaataccccctgcatccgataggcccgaactatcacaaacggaaaagcgatagcccaaaacactaagccccttt

SEQ ID NO: 8 - DNA, 131 bp, PRD1 bacteriophage
  source 1..131: mol_type = genomic DNA
aaataaatcaaaattttattcctatcgcttttccgtttgtgatagttcgggcctatcggatgcagggggtattgacagacggaaataaaaatgttaggggcgcgggtaggtggggaggggcacgtatcccc

SEQ ID NO: 9 - DNA, 2257 bp, synthetic construct
  source 1..2257: mol_type = other DNA
ggggatacgtgcccctccccacctacccgcgcccctaacatttttatttccgtctgtcaataccccctgcatccgataggcccgaactatcacaaacggaaaagcgatagcccaaaacactaagccccttttcatcatttcatttgtaagcagctgcagggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgctgcagtgggttgatgataccgctgccttactgggtgcattagccagtctgaatgacctgtcacgggataatccgaagtcaaaaaacccctcaagacccgtttagaggccccaaggggttatgctagttattgctcagcggtggcagcagccaactcttaatggtgatgatgatggtggctgcctttatacagttcatccataccgtgggtaatgcccgcggcggtaacaaattccagcagcaccatatgatcacgtttttcattcggatctttgctcagaacgctctgggtgctcagataatgattatccggcagcagcaccgggccatcaccaatcggggtattctgctgataatgatccgccagctgcacgctaccatcttccacgttgtgacggattttaaagttcgctttgatgccatttttctgtttatcggcggtaatatacacgttatggctgttgaaattatattccagtttatgacccagaatgttgccatcttctttaaaatcaatacctttcagttcaatgcggttcaccagggtatcgccttcaaatttaacttccgcacgggttttataggtgccatcatctttgaagctaatggtacgttcctgcacatagccttccggcatcgcgcttttaaagaaatcatggcgtttcatatgatccggatagcggctaaagcactgaacgccataatgcagggtggtcaccagggtcggccacggaaccggcagtttaccggtggtgcaaataaatttcagggtcagtttaccgttggtcgcatcaccttcgccttcgccacgaacgctaaatttatggccattcacatcaccatccagttccaccagaatcggcacaacgccggtaaacagttcttcacctttgctaaccatggttaattcctcctgttagcccaaaaaacgggtatggagaaaggtctgatcacattatacgagccgatgattaattgtcaagcgcaacgcaattaatgcgtgcaatagttatggtgaaaataaatcaaaattttattcctatcgcttttccgtttgtgatagttcgggcctatcggatgcagggggtattgacagacggaaataaaaatgttaggggcgcgggtaggtggggaggggcacgtatcccc

SEQ ID NO: 10 - DNA, 18 bp, synthetic construct
  source 1..18: mol_type = other DNA
ggggatacgtgcccctcc

SEQ ID NO: 11 - DNA, 18 bp, synthetic construct
  source 1..18: mol_type = other DNA
ggaggggcacgtatcccc

SEQ ID NO: 12 - DNA, 131 bp, PR5 bacteriophage
  source 1..131: mol_type = genomic DNA
ggggatacgtgcccctccccacctaccaccgcccctaacatttttatttccgtctgtcaatagcccctgcatccgataggcccgaactatcacaaacggaaaagcgatagcccaaaacactaagccccttt

SEQ ID NO: 13 - DNA, 132 bp, L17 or PR3 bacteriophage
  source 1..132: mol_type = genomic DNA
ggggatacgtgcccctccacccccaccgccaccccctaccaaaataatttccggttgtcaatagccccgcctcatcataggcccgaactatcacaaacggaaaagcgatagcccaaaacactaagccccttt

SEQ ID NO: 14 - DNA, 132 bp, PR4 bacteriophage
  source 1..132: mol_type = genomic DNA
ggggatacgtgcccctccaccgccacccgcaccccctaccaaaattatttccgtctgtcaataccccctgcatccaataggcccgaactatcacaaacggaaaagcgatagcccgaaacactaagccccttt

SEQ ID NO: 15 - DNA, 132 bp, PR772 bacteriophage
  source 1..132: mol_type = genomic DNA
ggggatacgtgcccctccaccgccacccgcaccccctaccaaaataatttccgtctgtcaataccccctgcatccgataggcccgaactatcacaaacggaaaagcgatagcccgaaacactaagccccttt

(19) United States
(12) Patent Application Publication
(10) Pub. No.: US 2026/0327635 A1
(43) Pub. Date: Oct. 22, 2026

(54) METHODS AND PRODUCTS FOR EVOLVING GENES

(71) Applicant: United Kingdom Research and Innovation, Swindon (GB)

(72) Inventors: Jason W. CHIN, Swindon (GB); Rongzhen TIAN, Swindon (GB); Fabian B.H. REHM, Swindon (GB); Jerome F. ZUERCHER, Swindon (GB); Dariusz CZERNECKI, Swindon (GB)

(21) Appl. No.: 19/485,408

(22) PCT Filed: May 23, 2024
(86) PCT No.: PCT/EP2024/064301

§ 371 Date: Nov. 18, 2025

(30) Foreign Application Priority Data
May 23, 2023 (GB) 2307706.8
Aug. 25, 2023 (GB) 2312968.7
Jan. 24, 2024 (GB) 2400921.9

(51) Int. Cl. C12N 15/73; C07K 14/005; C12N 9/12

(52) U.S. Cl. CPC C12N 15/73; C07K 14/005; C12N 9/1252; C12Y 207/07007; C12N 2710/00022; C12N 2795/10322

(57) ABSTRACT

The invention relates to cells comprising orthogonal DNA replication machinery, linear plasmids that may be replicated by said machinery, uses of said cells and said linear plasmids, methods of maintaining linear plasmids in cells, methods of evolving sequences of interest, and methods of making polypeptides or nucleic acids. The invention also relates to error-prone DNA polymerases, nucleic acids encoding said polymerases, and uses thereof.

### FIELD OF THE INVENTION

The invention relates to cells, such as bacterial cells, comprising orthogonal DNA replication machinery, linear plasmids that may be replicated by said machinery, uses of said cells and said linear plasmids, methods of maintaining linear plasmids in cells, methods of evolving sequences of interest, and methods of making polypeptides or nucleic acids. The invention also relates to error-prone DNA polymerases, nucleic acids encoding said polymerases, and uses thereof.

### BACKGROUND OF THE INVENTION

The evolution of new function in living organisms is the result of continuous genomic mutation and selection within a population. This process is slow, and the rate of evolution is fundamentally limited by the critical mutation rate, which sets the error threshold—the number of mutations a genome can tolerate before the organism is non-viable (1). Directed evolution commonly sidesteps the limitation on in vivo mutation rate by generating genetic diversity in vitro (2), but this does not enable the continuous evolution of genes within an organism. The mutation rate of cells can be transiently increased, but high levels of untargeted mutation leads to a catastrophic mutational load on the genome and are unsustainable. Genes inserted in viral genomes can be mutated by iteratively infecting new mutagenic cells (3-6), this approach sidesteps the challenge of increasing the rate of sustained mutation on genes in cells and can be extended to select for some phenotypes (7). However, this strategy is limited to evolving genes that are small enough to be packaged into viruses, limited to selecting phenotypes that can be coupled to infectivity and, selection occurs in cells under conditions of replicative stress, which may further limit the cellular phenotypes that can be explored.

Strategies that direct mutations to specific, targeted DNA sequences within cells—without substantially increasing the genomic mutation rate—offer the possibility of driving accelerated, sustainable-, continuous-, cellular-evolution of target sequences (8-17). Pioneering work has taken advantage of an existing natural linear plasmid, that functions in the yeast cytosol and is replicated by a dedicated DNA polymerase that does not copy the yeast genome, as a natural orthogonal replication system (12, 13). By recombining target genes into this existing linear plasmid system in yeast, and generating mutagenic orthogonal DNA polymerases, a continuous evolution system was developed in this host. This system has been used to evolve metabolic pathways and antibodies, and provided key insights into evolutionary trajectories (12, 13, 18). However, the system cannot be used for engineering bacterial genetic elements, and requires tedious steps to engineer the established replicons in vivo and to cure competing unmodified replicons. Moreover, the doubling time of yeast makes this system theoretically slower than systems based on bacteria. Recent work has shown that target genes can also be recombined into a natural linear plasmid (from a lysogenic phage) in B. thuringiensis, and this system can also be used to generate a mutagenic orthogonal replication system (14). However, there are very limited genetic tools in this organism and the host is not widely used or well characterized. Moreover, current mutagenic orthogonal replication systems, and viral evolution systems, have strong biases, towards certain transversions or transitions, in the mutations they introduce; this biases the evolutionary pathways, and therefore the potential phenotypes, that may be accessed by current methods.

E. coli is the workhorse of molecular biology and is widely used in both fundamental discovery science and industrial production (19). It is the best characterized organism and many of its biochemical pathways have been characterized in detail. It has a rapid doubling time, is a preferred host for gene cloning and protein expression, and a vast repertoire of genetic tools have been developed for this organism over many years. An outstanding challenge over the past decade has been to discover a stable orthogonal replication system that operates in E. coli, and thereby enable accelerated continuous evolution in this host. However, despite substantial effort, no stable orthogonal replication system has been discovered in E. coli.

Continuous directed evolution is a powerful tool to obtain proteins with desired properties. In contrast to standard directed evolution experiments the continuous workflow allows generation of diversity and selection without user interference mimicking a Darwinian process over multiple generations. Currently there are two major systems for continuous directed evolution i) Phage Assisted Continuous Evolution (PACE) and ii) Orthogonal DNA Replication System (OrthoRep) in yeast (see, e.g. Ravikumar et al., Nature Chemical Biology, Vol 10, P175-177, March 2014). While PACE suffers from several limitations (cargo size, selection coupling to phage life cycle, etc), OrthoRep offers a less biased platform. However, the major drawback of OrthoRep is its reliance on yeast which has a slow doubling time compared to bacterial systems.

Attempts have been made to develop an orthogonal system for continuous directed evolution in bacteria, for instance based on the bacteriophage 29, but have not been successful (see, e.g., Kearns et al., “DiversiPhi29: an orthogonal system for the continuous directed evolution of genes in vivo”).

CN115772533A reports a continuous evolution system that makes use of a lysogenic phage that exists in the form of plasmids. This system is limited to Bacillus thuringiensis, which is not a common bacterial host.

There is, therefore, a need for an orthogonal DNA replication system that is suitable for use in more appropriate host cells.

### SUMMARY OF THE INVENTION

In a first aspect, there is provided a cell comprising orthogonal DNA replication machinery, wherein the orthogonal DNA replication machinery is from or derived from a lytic phage of the Tectiviridea family or a lytic φ29-like virus of the Podoviridae family.

The cell may be a bacterial cell, such as an E. coli cell. One or more non-orthogonal episomes that are capable of being replicated by the endogenous DNA polymerase may encode all components of the orthogonal DNA replication machinery. At least one, a plurality, or all components of the orthogonal DNA replication machinery may be encoded by the cell genome. The orthogonal DNA replication machinery may be capable of replicating an orthogonal episome that does not comprise at least one, a plurality, or all components of the orthogonal DNA replication machinery. The orthogonal DNA replication machinery may replicate DNA with a lower fidelity than the cell's endogenous DNA replication machinery.

The orthogonal DNA replication machinery may be from or derived from a Tectiviridae family member. The orthogonal DNA replication machinery may be from or derived from any of phage PRD1, phage PR3, phage PR4, phage PR5, phage L17, phage PR722, and phage BCE1. The orthogonal DNA replication machinery may comprise a gene encoding a Podoviridae or Tectiviridae terminal protein (TP) and a gene encoding an orthogonal DNA polymerase (ODNAP). The orthogonal DNA replication machinery may comprise a gene encoding a Podoviridae or Tectiviridae terminal protein (TP), a gene encoding an orthogonal DNA polymerase (ODNAP), a gene encoding a Podoviridae or Tectiviridae single-stranded DNA-binding protein (SSB), and optionally a gene encoding a Podoviridae or Tectiviridae double-stranded DNA-binding protein (DSB). The ODNAP may be a Podoviridae or Tectiviridae ODNAP or an engineered Podoviridae or Tectiviridae ODNAP.

The cell may comprise sequence encoding a protein that is at least 80%, 85%, 90%, 95%, 97%, 98%, 99%, or 100% similar or identical to any one, two, three, or four of SEQ ID NOs: 1, 2, 3, and 4. At least one, a plurality, or all components of the orthogonal DNA replication machinery may be encoded by a single operon. The cell may comprise a gene encoding Gam protein and may express said protein.

The cell may comprise an episome that is capable of being replicated by the orthogonal DNA replication machinery. The episome may comprise a 5′ inverted terminal repeat (ITR) and a 3′ ITR, wherein the 5′ ITR and the 3′ ITR are compatible with the orthogonal DNA replication machinery. The episome may comprise a 5′ ITR that is at least 80%, 85%, 90%, 95%, 97%, 98%, 99%, or 100% identical to SEQ ID NO: 7, or a truncation thereof, and/or a 3′ ITR that is at least 80%, 85%, 90%, 95%, 97%, 98%, 99%, or 100% identical to SEQ ID NO: 8, or a truncation thereof. The episome may comprise a 5′ ITR that is at least 80%, 85%, 90%, 95%, 97%, 98%, 99%, or 100% identical to SEQ ID NO: 10 and/or a 3′ ITR that is at least 80%, 85%, 90%, 95%, 97%, 98%, 99%, or 100% identical to SEQ ID NO: 11. The episome may be a linear plasmid and may comprise a sequence-of-interest.

The cell may comprise a linear plasmid of the second aspect or a circular nucleic acid molecule of the sixth aspect.

In a second aspect, there is provided a linear plasmid comprising a 5′ ITR and a 3′ ITR, each of which is compatible with DNA replication machinery from or derived from a lytic phage of the Tectiviridea family or a lytic φ29-like virus of the Podoviridae family.

In an embodiment, the 5′ ITR and 3′ ITR are compatible with PRD1 bacteriophage DNA replication machinery. The 5′ ITR may be at least 80%, 85%, 90%, 95%, 97%, 98%, 99%, or 100% identical to SEQ ID NO: 7, or a truncation thereof and/or the 3′ ITR may be at least 80%, 85%, 90%, 95%, 97%, 98%, 99%, or 100% identical to SEQ ID NO: 8, or a truncation thereof. The episome may comprise a 5′ ITR that is at least 80%, 85%, 90%, 95%, 97%, 98%, 99%, or 100% identical to SEQ ID NO: 10 and/or a 3′ ITR that is at least 80%, 85%, 90%, 95%, 97%, 98%, 99%, or 100% identical to SEQ ID NO: 11. The plasmid may not encode one, two, three, or four of a TP, a DNA polymerase, an SSB, and optionally a DSB. The plasmid may comprise a sequence-of-interest. The sequence-of-interest may not be a sequence endogenous to Podoviridae or Tectiviridae family bacteriophages.

In a third aspect, there is provided a method of maintaining a linear plasmid, wherein the method comprises: i) providing a cell according to the first aspect or transferring a linear plasmid according to the second aspect to a cell according to the first aspect; and ii) incubating the cell under conditions conducive to growth. The transferring of a linear plasmid may be the excision of a linear plasmid of the second aspect from a circular nucleic acid molecule of the sixth aspect within a cell of the first aspect.

In a fourth aspect, there is provided use of a cell according to the first aspect, a linear plasmid according to the second aspect, or a circular plasmid of the sixth aspect for evolving a sequence-of-interest.

In an embodiment, there is provided a method of evolving a sequence-of-interest, wherein the method comprises: i) providing a cell according to the first aspect or transferring a linear plasmid according to the second aspect to a cell according to the first aspect, wherein the episome or linear plasmid comprises a sequence-of-interest and wherein the orthogonal DNA replication machinery replicates DNA with a lower fidelity than the cell's endogenous DNA replication machinery; ii) incubating the cell under conditions conducive to growth; and iii) exposing the cell to a selection condition. The transferring of a linear plasmid may be the excision of a linear plasmid of the second aspect from a circular nucleic acid molecule of the sixth aspect within a cell of the first aspect. The method may optionally comprise iv) identifying the sequence of a sequence-of-interest from a cell meeting the selection condition. The method may optionally comprise v) making a polypeptide or nucleic acid encoded by the identified sequence-of-interest.

In a fifth aspect, there is provided a method of making a polypeptide or nucleic acid, wherein the method comprises: i) providing a sequence-of-interest identified by a use or method of the fourth aspect, and ii) producing a polypeptide or nucleic acid according to said sequence.

In a sixth aspect, there is provided a circular nucleic acid molecule comprising the sequence of a linear plasmid of the second aspect.

In a seventh aspect, there is provided a DNA polymerase comprising any of the mutations disclosed herein.

In an eight aspect, there is provided a nucleic acid sequence encoding a DNA polymerase of the seventh aspect.

In a ninth aspect, there is provided use of a DNA polymerase of the seventh aspect.

### BRIEF DESCRIPTION OF THE DRAWINGS

FIG. 1. An illustrative embodiment referred to as “Escherichia coli Orthogonal DNA Replication System” (EcORep). a) A schematic illustrating EcORep. b) An exemplary operon and linear plasmid (LP) arrangement. EcORep was constructed based on the linear genome and its replication machinery of E. coli phage PRD1 from the Tectiviridae family. To establish the system, first the four essential genes from the phage genome for orthogonal replication were organised in one operon and integrated in the E. coli genome. Various promoters were tested for controlling the operon. Then the linear plasmid was designed based on the structure of the phage genome. c), d), and e) The linear plasmid was electroporated into cells encoding the LP replication machinery. We confirmed the existence of the LP based on phenotype (antibiotic resistance and GFP fluorescence), genotype (PCR verification, next-generation sequencing) and extraction of the LP (with or without protease K). For the strain that expressing LP replication machinery under the control of an inducible IPTG promoter and harbouring the LP, the GFP expression level increases with increasing IPTG concentrations, which is also indirect evidence of a change in LP copy number. f) Testing the expression level of GFP encoded by LP when the PRD1 gene cluster was controlled by different promoters.

FIG. 2. Testing the stability of the EcORep system. a) Illustration of the linear plasmid and the circular plasmid. Both plasmids express the same genes using the same sequence. For LP, we tested the stability of it when the PRD1 gene cluster was controlled by 8 different promoters. b) Illustration of the plasmid stability test procedure and results. The cultures of the strains were serially diluted at 1/1000 (approximately 2¹⁰) and cultured for 12 h both with or without antibiotic selection. Flow cytometry was used to determine the proportion of cells expressing green fluorescent protein (GFP) and then to further represent the proportion of cells with plasmids. Gen, generations. We found the LP to be stably maintained over ~90 generations with antibiotics, which is much more stable than the circle plasmids with a ColE1 replication origin.

FIG. 3. Establishing a synthetic orthogonal replicon in E. coli. (A) PRD1 undergoes uncontrolled replication upon infecting E. coli and rapidly lyses host cells. Constructing a synthetic operon enables controlled replication of an orthogonal replicon. TP, terminal protein; O-DNAP, orthogonal DNA polymerase; SSB, single-stranded DNA binding protein; DSB, double-stranded DNA binding protein; ITR, inverted terminal repeat (in yellow). (B) We combined genes for replication of the orthogonal replicon to generate a synthetic replication operon. (C) Orthogonal replicons can be established by electroporating into E. coli cells harboring a genomic synthetic replication operon. The linear Kan^{R}-GFP replicon consists of flanking 110 bp ITR, a kanamycin resistance gene and a GFP gene. (D) Efficiency of establishing orthogonal replicons by electroporating 3 μg Kan^{R}-GFP PCR product into 100 μL (~10⁹ cells). Expression of gam, SSB and DSB genes from a helper plasmid increased efficiency. (n=3, error bars are ±s.d.) (E) Extraction of the Kan^{R}-GFP orthogonal replicon from cells. Proteinase K addition was needed to remove the terminal proteins. The control is a PCR product. (F) Essentiality of genes in the synthetic replication operon for establishing the orthogonal replicon. (G) A 16.5 kb orthogonal replicon. Shown is Illumina sequencing read coverage.

FIG. 4. Orthogonal replicons are stably maintained for hundreds of generations. (A) Stability of the Kan^{R}-GFP orthogonal replicon over 300 generations with or without kanamycin, as assessed by maintenance of GFP fluorescence via flow cytometry. The synthetic replication operon was under the control of a PdnaKJpromoter. (B) The inverted terminal repeat origins were iteratively truncated to establish a minimal origin for an orthogonal replicon. A single 18 bp repeat is shown in orange and the rest of the inverted terminal repeat in yellow. (C) Stability over 100 generations of the truncated orthogonal replicons shown in (B) in the presence of kanamycin, as assessed by maintenance of GFP fluorescence via flow cytometry. (D) Multiple distinct orthogonal replicons were sequentially transformed and co-maintained under selection. (E) Stability, over 100 generations, of two or three co-maintained orthogonal replicons, as shown in (D), in the presence of the corresponding antibiotics, as assessed by maintenance of GFP fluorescence via flow cytometry. For the doubly transformed cells, O-RA corresponds to an orthogonal replicon carrying Amp^{R}. For all experiments n=3, data are shown as mean±s.d.

FIG. 5. Control of orthogonal replicon copy number over a 465-fold range. (A) Control of the orthogonal replicon copy number is achieved by inducing expression of the synthetic replication operon via IPTG addition, or by downregulating its expression via an arabinose inducible dCas9 targeted to the IPTG-responsive Ptac promoter. (B) Orthogonal replicon copy number, as determined by qPCR, and GFP fluorescence (normalized to OD₆₀₀) were measured at different arabinose or IPTG concentrations. (C) Correlation between Kan^{R}-GFP orthogonal replicon copy number and GFP fluorescence. Data from (B) was replotted for (C). For all experiments n=3, data are shown as mean±s.d.

FIG. 6. Mutagenic orthogonal DNA polymerases selectively mutate the orthogonal replicon, but not the genome. Determination of genomic or orthogonal replicon mutation rate (p, s.p.b.) for the O-DNAP and its engineered variants. The mutation rate was measured after 10 generations with fluctuation tests. For assessment of the orthogonal replicon mutation rate, we used an orthogonal replicon-encoded Cm^{R} gene with a TAG stop codon at position 38 and the O-DNAP variants were expressed from genomically integrated synthetic replication operons. For assessment of the genome mutation rate, we used a genomically-encoded Cm^{R} gene with a TAG stop codon at position 38 and the O-DNAP variants were expressed from p15A plasmids via rhamnose induction. For all experiments n=12, data are shown as mean±upper/lower 95% bounds.

FIG. 7. Accelerated continuous evolution of a 150-fold increase in tigecycline resistance in 12 days. (A) Analysis of an evolved pool (after 14 passages) of cells carrying the Kan^{R}-TetA orthogonal replicon. Shown is the pool for replicate 10 performed with the N71D O-DNAP; FIG. 20 shows other replicates. (B) AlphaFold2 model of TetA. Gradient indicates the mutational frequency of each residue. (C) Validation of evolved tetA on a ColE1 plasmid. Shown is Mut_3 from the replicate 10 pool; FIG. 22 shows other mutants. Either the EM7 promoter or the evolved promoter (pMut_3) were used to drive expression. WT tetA or a previously reported mutant were assessed for comparison. For all experiments n=4, data are shown as mean±s.d.

FIG. 8. Accelerated continuous evolution of a 10³-fold increase in GFP fluorescence in less than five days. (A) To select for brighter variants of a Kan^{R}-GFP orthogonal replicon, we iteratively isolated the brightest 0.1% of cells via fluorescence-activated cell sorting. Replicate 12 from the Y127A O-DNAP is shown; FIG. 25 shows other replicates. (B) Structure of GFP (2B3P). Gradient indicates the mutational frequency of each residue. (C) Validation of evolved GFP and/or evolved promoter variants on a ColE1 plasmid. PMut_1 and Mut_1 were obtained with the N71D O-DNAP, PMut_2 and Mut_2 were obtained with the Y127A O-DNAP. FIG. 27 shows other mutants. For all experiments n=4, data are shown as mean±s.d.

FIG. 9. Phylogenetic analysis of Tectiviridae family phages that prey on E. coli. (A) Phylogenetic tree. (B) Alignment of the left inverted terminal repeat of Tectiviridae family phages. The first 18 bp are conserved. The sequences shown are SEQ ID NO: 7 (PRD1) and SEQ ID NOs: 12 to 15.

FIG. 10. The helper plasmid is readily cured after establishing the orthogonal replicon. (A) Establishing an orthogonal replicon is enhanced by a helper plasmid encoding Gam and the PRD1 SSB and DSB genes. After the replicon is established, the helper plasmid is readily cured. (B) Colony PCRs that amplify from the helper plasmid backbone were used to assess whether the plasmid was cured after plating without selection for helper plasmid maintenance and the addition of 1 mg mL⁴ streptomycin for negative selection against the rpsL gene on the helper plasmid backbone. All seven colonies were cured of the helper plasmid.

FIG. 11. Flow cytometry gating strategy of the orthogonal replicon when the synthetic replication operon is controlled by different promoters. (A) Flow cytometry gating strategy for non-fluorescent negative control cells. (B) Flow cytometry gating strategy for fluorescent positive control cells. These were the cells obtained immediately after establishing a Kan^{R}-GFP replicon (as shown as generation 0 with antibiotic maintenance in FIG. 2A).

FIG. 12. Copy number of minimal orthogonal replicons. Orthogonal replicon copy number, as determined by qPCR, and GFP fluorescence (normalized to OD₆₀₀) were measured for the origin-truncated Kan^{R}-GFP replicons. (n=4 for fluorescence and n=3 for qPCR-determined copy number, data are shown as mean±s.d.)

FIG. 13. Control of orthogonal replicon copy number via induction of the synthetic replication operon. (A) GFP fluorescence (normalized to OD₆₀₀) from orthogonal replicon- or circular plasmid-encoded (ColE1, 40-50 copies per cell) GFP at different IPTG concentrations; in both the orthogonal replicon and the circular plasmid the GFP is expressed from a constitutive Ptac promoter. The Ptac-lac operon that controls the synthetic replication operon is IPTG inducible. (n=4, data are shown as mean±s.d.) (B) Orthogonal replicon copy number, as determined by qPCR, at different IPTG concentrations. (n=3, data are shown as mean±s.d.)

FIG. 14. Optimizing dCas9-mediated control of orthogonal replicon copy number. (A) Control of the orthogonal replicon copy number is achieved by inducing expression of the synthetic replication operon via IPTG addition, or by downregulating its expression via an arabinose inducible dCas9 targeted to the IPTG-responsive Ptac promoter. (B) Assessment of three sgRNAs to guide dCas9 to the IPTG-responsive Ptac promoter, and their capacity to reduce Kan^{R}-GFP orthogonal replicon copy number as determined by the proportion of kanamycin susceptible cells. (C) Assessment of three sgRNAs to guide dCas9 and their capacity to reduce Kan^{R}-GFP orthogonal replicon copy number as determined by GFP fluorescence at a range of IPTG or arabinose concentrations. sgRNA 2 was selected due to its concentration-dependent control of orthogonal replicon copy number.

FIG. 15. Different promoters for the synthetic replication operon lead to different orthogonal replicon copy numbers. Kan^{R}-GFP orthogonal replicon copy number, as determined by qPCR, and GFP fluorescence (normalized to OD₆₀₀) were measured for synthetic replication operons under the control of different promoters.

FIG. 16. Controlling orthogonal replicon copy number via expression of TP and O-DNAP from a plasmid. Control of orthogonal replicon copy number is achieved by inducing expression of the synthetic replication operon via IPTG addition, or by downregulating its expression via an arabinose inducible dCas9 targeted to the IPTG-responsive Ptac promoter. A plasmid (p15A origin) expressing both the TP and DNAP under rhamnose control facilitates increased orthogonal replicon copy number following suppression of copy number by dCas9-mediated repression of the genomically-encoded synthetic replication operon. Orthogonal replicon copy number, as determined by qPCR, and GFP fluorescence (normalized to OD₆₀₀) were measured for cells where orthogonal replicon copy number was suppressed via addition of arabinose and a range of rhamnose concentrations were added to overexpress TP (A) or DNAP (B), or a combination of both (C). Only co-expression of TP and DNAP was able to increase orthogonal replicon copy number following copy number reduction via dCas9. (D) Orthogonal replicon copy number, as determined by qPCR, and GFP fluorescence (normalized to OD₆₀₀) were measured for the TP and DNAP co-expression system shown in (C) with different combinations of IPTG, rhamnose and arabinose, as indicated. For all experiments n=3, data are shown as mean±s.d.

FIG. 17. Mutation rates and orthogonal replicon copy numbers for all O-DNAP mutants. (A) AlphaFold2-predicted model of PRD1 DNAP, displaying the typical Klenow fold of family B DNA polymerases. Residues mutated in error-prone polymerase variants (D17A, T201, H70R, N71D, D76A, Y127A, L341R) are shown in stick representation, labelled and highlighted in pink. Six of them concentrate around the 3′-5′ exonuclease catalytic site; L341R is located in the fingers subdomain. (B) Determination of orthogonal replicon mutation frequency. The proportion of chloramphenicol-resistant cells was measured for an orthogonal replicon, replicated by the indicated O-DNAP mutant, carrying a CmR gene with a TAG stop codon at position 38. Cells were plated after 10 generations of growth in culture. (n=4, data are shown as mean±s.d.) (C) Determination of orthogonal replicon copy number, via qPCR, as maintained by the indicated O-DNAP mutants. (n=3, data are shown as mean±s.d.) (D) The number of mutations per generation per base pair (mutation rate p1, s. p. b.) was calculated from (A) and the mean values from (B) for each DNAP mutant. (n=4, data are shown as mean±s.d.)

FIG. 18. Mutational spectra for the N71D and Y127A O-DNAP mutants. (A) Determination of the orthogonal replicon mutation rate (p, s.p.b.) for the O-DNAP and its engineered variants. The O-DNAPs were expressed from p15A plasmids via rhamnose induction and we repressed expression of the genomically-encoded WT O-DNAP using dCas9. The mutation rate was measured after 10 generations with fluctuation tests using an orthogonal replicon-encoded Cm^{R} gene with a TAG codon at position 38. (n=12, data are shown as mean±upper/lower 95% bounds). (B) Determination of the mutational spectra for the N71D and Y127A O-DNAP mutants.

FIG. 19. Overview of the tetA accelerated continuous evolution for tigecycline resistance. (A) Cells were passaged in 24-well plates once the OD₆₀₀ reached 0.5 or higher. O-replicon copy numbers for the two O-DNAP mutants with or without rhamnose induction are indicated. We iteratively increased tetracycline 50 concentration at each passage, as shown in (B). We then spotted each of the 24 pools (resulting from the 12 replicates with the N71D and Y127A O-DNAP mutants) on agar plates containing tigecycline concentrations up to 150 μg ml⁻¹ and also diluted each pool on to agar plates containing 35 μg ml⁻¹ tigecycline to pick single colonies for sequencing and sub-cloning; all agar plates lacked rhamnose. We note that this evolution was conducted without the dCas9-mediated repression of genomically-integrated WT O-DNAP expression.

FIG. 20. Tigecycline and tetracycline resistance of pools of cells harboring evolved tetA orthogonal replicons. After 14 passages of mutagenic replication, via the N71D (replicates 1-12) or Y127A (replicates 1-12) O-DNAP mutants, with increasing concentrations of tigecycline, pools of evolved cells were spotted on the indicated tigecycline or tetracycline concentrations. Cells carrying an unevolved wild-type tetA replicon were included as a control. The identical images for replicate 10 are also shown in FIG. 7A.

FIG. 21. Observed mutations in the accelerated continuous evolution of tetA. (A) Frequency of the indicated promoter and 5′ UTR mutations. (B) Frequency of the synonymous mutations. (C) Frequency of the non-synonymous mutations. For (A-C), five colonies from 12 replicates for each of two O-DNAPs (120 colonies total) of the evolution were sequenced. Only mutations that occurred at a frequency of 4% or higher are shown.

FIG. 22. Validation of evolved tetA variants. Cells harbouring a plasmid (ColE1 origin) with the evolved tetA variants, wild type tetA, or a previously reported mutant, under control of an EM7 promoter or the corresponding evolved promoter, were spotted on a range of tigecycline or tetracycline concentrations, as indicated. The identical images for Mut_3 are shown FIG. 7C.

FIG. 23. Comparison of our WT GFP and sfGFP fluorescence (normalized to OD₆₀₀) under the control of a rhamnose-inducible promoter. n=3, data are shown as mean±s.d.; individual data points are shown as dots.

FIG. 24. Overview of the GFP accelerated continuous evolution for brighter fluorescing cells. O-replicon copy numbers for the two O-DNAP mutants with or without rhamnose and arabinose induction are indicated.

FIG. 25. Overview of the parallel accelerated continuous evolution for GFP fluorescence. The GFP fluorescence before and after each of the three selection rounds is shown for all 12 replicates for the evolved in parallel. Identical data for replicate 12 from the Y127A O-DNAP evolution is shown in FIG. 8A.

FIG. 26. Observed mutations in the accelerated continuous evolution of GFP. (A) Frequency of the indicated promoter and 5′ UTR mutations. (B) Frequency of the synonymous mutations. (C) Frequency of the non-synonymous mutations. For (A-C), five colonies from 12 replicates for each of two O-DNAPs (120 colonies total) of the evolution were sequenced. Only mutations that occurred at a frequency of 4% or higher are shown. The two most frequent mutations were L43M (39.2%) and H66L (36.7%), and the occurrence of these mutations was mutually exclusive. Whereas the H66L mutation directly converts the chromophore to L₆₆-Y₆₇-G₆₈, which has previously been described to display four- to sixfold greater green fluorescence than the canonical GFP chromophore (S₆₆-Y₆₇-G₆₈), the alternative L43M mutation has not been described and appears to be compensatory—the L43 sidechain points into the beta barrel, towards the chromophore, and thus its substitution to M might enable interactions that could culminate in improved chromophore maturation or stability.

FIG. 27. Testing the evolved promoter and GFP variants. Promoter or GFP variants, or a combination of both, were cloned into ColE1 plasmid backbones and the culture fluorescence, normalized to OD₆₀₀, was measured. Some of the data is also shown in FIG. 8C. (n=4, data are shown as mean±s.d.; individual data points are shown as dots)

FIG. 28. Design of a system for in vivo cleavage of circular DNA to generate an orthogonal episome.

FIG. 29. ODNAP mutants with higher error-rates.

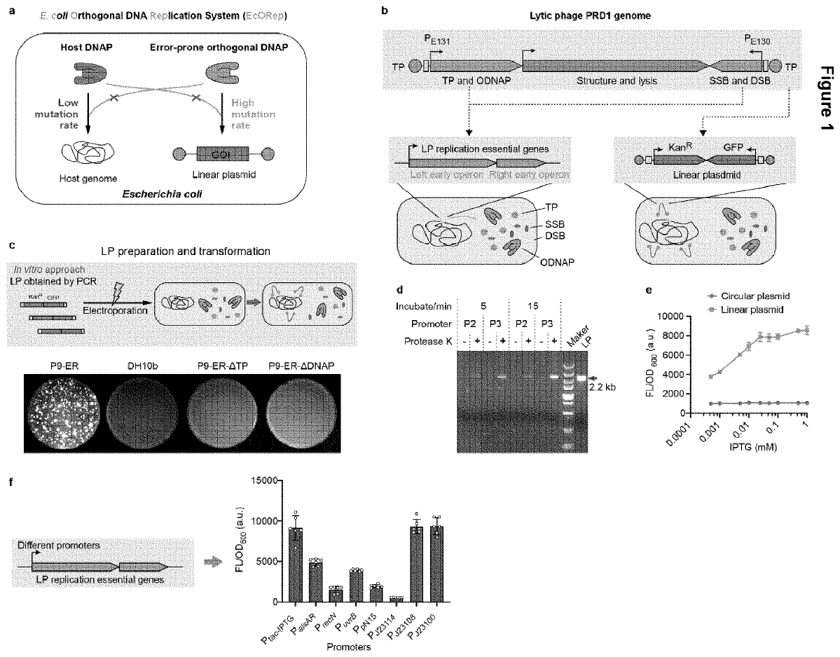

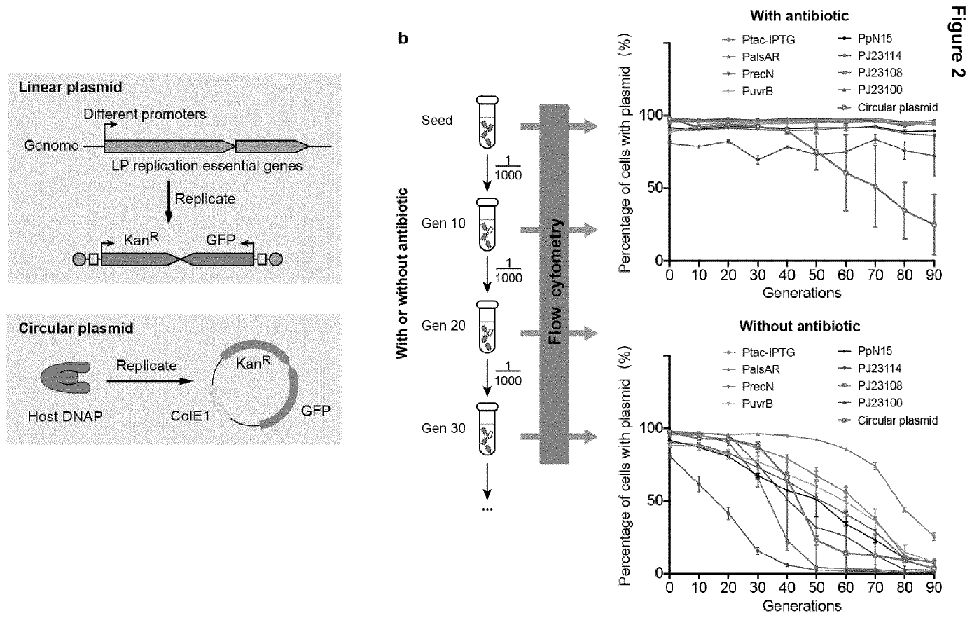

### DETAILED DESCRIPTION

Provided herein is a self-contained orthogonal DNA replication system. The inventors have discovered that lytic phages from the Podoviridae or Tectiviridea family comprise replication machinery that can be used to maintain an episome in host cells, wherein the episome does not need to encode the proteins that form the replication machinery. Thus, the inventors demonstrate that one or more, or all, components of the orthogonal replication machinery may be encoded by the host cell, for instance the host cell's genome, rendering the cell capable of maintaining orthogonal episomes. The inventors demonstrate that this system is functional in bacterial cells.

This finding provides a system that allows continuous directed evolution techniques to be applied to a sequence-of-interest without affecting the endogenous genes of the host.

Thus, in a first aspect, there is provided a cell comprising orthogonal DNA replication machinery, wherein the orthogonal DNA replication machinery is from or derived from a lytic phage of the Tectiviridea family or a lytic φ29-like virus of the Podoviridae family.

Orthogonal DNA replication machinery is not capable of replicating DNA that is endogenous to the host cell or is less capable of replicating DNA that is endogenous to the host cell compared to the host DNA replication machinery, but is capable of replicating DNA that is compatible with the orthogonal DNA replication machinery.

The reduced ability to replicate DNA that is endogenous to the host cell may mean that, when active in a host cell, error-prone orthogonal DNA replication machinery does not introduce mutations into the host cell genome at a significant rate. For instance, the host genome mutation rate may be less than 10%, less than 5%, less than 2%, less than 1%, less than 0.5%, or less than 0.1% of the mutation rate for DNA that is compatible with the orthogonal DNA replication machinery. The reduced ability to replicate DNA that is endogenous to the host cell may mean that, when active in a host cell, error-prone orthogonal DNA replication machinery does not introduce mutations into the host cell genome. The reduced ability to replicate DNA that is endogenous to the host cell may mean that, when active in a host cell, the orthogonal DNA replication machinery is responsible for less than 10%, less than 5%, less than 2%, less than 1%, less than 0.5%, or less than 0.1% of replication of endogenous DNA.

The cell of the first aspect may be a bacterial cell. For example, the bacterial cell may a proteobacteria with P, N, or W incompatibility plasmids. The bacterial cell may be from the genus or family Acinetobacter, Enterobacteria, Pseudomonas, Vibrio, or Escherichia. In a particular embodiment, the host cell is an E. coli cell. Suitable E. coli cells include K-12, MG1655, BL21, BL21(DE3), AD494, Origami, HMS174, BLR(DE3), HMS174(DE3), Tuner(DE3), Origami2(DE3), Rosetta2(DE3), Lemo21(DE3), NiCo21(DE3), T7 Express, Shuffle Express, C41(DE3), C43(DE3), and m15 pREP4 or derivatives thereof (Rosano, G. L. and Ceccarelli, E. A., 2014. Frontiers in microbiology, 5, p. 172). In particular, the cell may be MG1655 or BL21, or a derivative thereof. MG1655 is considered as the wild type strain of E coli. The GenBank ID of genomic sequence of this strain is U00096. BL21 is widely available commercially. For example, it can be purchased from New England BioLabs with catalog number C2530H. In an example, the cell is not a Bacillus thuringiensis cell.

The cell of the first aspect is a viable cell. The cell does not comprise genes that encode the sufficient lytic protein components of the phage from which the orthogonal DNA replication machinery is derived to result in cell lysis.

In some embodiments, the cell does not comprise any genes that encode lytic protein components of the phage from which the orthogonal DNA replication machinery is derived. In a particular embodiment, the cell does not comprise any genes that encode structural or lytic protein components of the phage from which the orthogonal DNA replication machinery is derived.

One or more components of the orthogonal DNA replication machinery may be encoded by a non-orthogonal episome within the cell (i.e. an episome that can be maintained by the endogenous DNA replication machinery of the host cell but not by the orthogonal DNA replication machinery). For instance, at least one, a plurality, or all components of the orthogonal DNA replication machinery may be encoded by the genome of the cell. This provides a cell that contains the necessary machinery to replicate compatible orthogonal DNA. The cell may comprise one or more non-orthogonal plasmid, or other non-genomic episome, that encodes at least one, a plurality, or all components of the orthogonal DNA replication machinery. Such plasmids may be referred to as “helper plasmids”. In some embodiments, the components of the orthogonal DNA replication machinery are split between the genome of the cell and one or more non-orthogonal non-genomic episomes. For instance, one or more components of the orthogonal DNA replication machinery may be encoded by the cell genome and one or more components of the orthogonal DNA replication machinery may be encoded by one or more helper plasmid.

The orthogonal DNA replication machinery may be encoded within a single operon. Thus, the gene or genes of the orthogonal DNA replication machinery may be operably linked to a single promoter. The genes encoding the orthogonal DNA replication machinery may be referred to as recombinant genes. These genes are derived from phage but are encoded on non-orthogonal episomes within the cell, i.e. episomes that cannot be replicated by the wild-type phage DNA polymerase.

The term “episome” has its ordinary meaning in the art, for example any accessory extrachromosomal replicating genetic element that can exist either autonomously or can become integrated with the chromosome.

Orthogonal DNA replication systems are suitable for evolving sequences because they allow target sequences to be replicated with a lower fidelity than the genes endogenous to the host. Thus, the target sequences are mutated at a higher frequency than the endogenous genes. Therefore, in an embodiment, the orthogonal DNA replication machinery replicates DNA with a lower fidelity than the host cell's endogenous DNA replication machinery. This means that the orthogonal DNA replication machinery is more error prone when compared to the endogenous DNA replication machinery. Techniques to measure and compare the fidelity of replication by DNA polymerases are known in the art.

As discussed herein, the inventors have identified that DNA replication machinery of $29-like lytic phages from the Podoviridae family or lytic phages of the Tectiviridea family is suitable for the creation of orthogonal DNA replication systems. The term “lytic phage”, as used herein, refers to bacteriophages that only have a lytic cycle and do not have a lysogenic cycle. Thus, the term “lytic phage” does not encompass lysogenic phages and does not encompass temperate phages with a “plasmidial” prophage state. Publications describing phages include: Meijer et al. (Phi29 family of phages. Microbiol. Mol. Biol. Rev. 65, 87-261, 2001), Saren et al. (A Snapshot of viral evolution from genome analysis of the Tectiviridae family. J. Mol. Bio. 350, 427-440, 2005), and Gillis et al. (Phages preying on Bacillus anthracis, Bacillus cereus, and Bacillus thuringiensis. Past, present and future.

Viruses 6, 2623-2672, 2014), each of which is incorporated herein by reference.

Examples of lysogenic phages that are not encompassed by the term “lytic phage” include the phages GA-1, GILO1, GIL02, GIL16, Bam35, MZTP02, AP50, Wip1, TP21, TP21-L, and TP21-H. Thus, the orthogonal DNA replication machinery comprised by cells of the first aspect is not derived from lysogenic phages such as GA-1, GILO1, GIL02, GIL16, Bam35, MZTP02, AP50, Wip1, TP21, TP21-L, or TP21-H.

In a particular embodiment, the orthogonal DNA replication machinery is from or derived from a Tectiviridae family member. In a preferred embodiment, the orthogonal DNA replication machinery is from or derived from any of phage PRD1, phage PR3, phage PR4, phage PR5, phage L17, phage PR722, and phage BCE1.

The bacteriophages from which the orthogonal replication machinery of the invention is derived comprise two genes that together provide DNA replication machinery capable of maintaining an orthogonal episome within the cell. These two genes are a gene encoding a terminal protein (TP) and a gene encoding an DNA polymerase. As discussed further herein, embodiments where the cell comprises only these two genes are particularly relevant when the orthogonal episome is provided as a complex that already comprises the TP. The genes may be naturally occurring genes or may comprise one or more substitutions, insertions, or deletions. The genes may have at least 80%, 85%, 90%, 95%, 97%, 98%, 99%, or 100% similarity or identity to a naturally occurring gene from a 029-like lytic phage from the Podoviridae family or a lytic phage from the Tectiviridea family. The genes may have at least 80%, 85%, 90%, 95%, 97%, 98%, 99%, or 100% similarity or identity to a naturally occurring gene from a lytic phage that is a member of the Tectiviridae family. The genes may have at least 80%, 85%, 90%, 95%, 97%, 98%, 99%, or 100% similarity or identity to a naturally occurring gene from any of phage PRD1, phage PR3, phage PR4, phage PR5, phage L17, phage PR722, or phage BCE1.

The bacteriophages from which the orthogonal replication machinery of the invention is derived comprise three genes that together form the components of the DNA replication machinery for establishing an orthogonal episome (as mentioned above, fewer genes are required to maintain an already established episome). These are a gene encoding a TP, a gene encoding an DNA polymerase, and a gene encoding a single-stranded DNA-binding protein (SSB). The bacteriophages also include an optional further component that may form part of the orthogonal replication machinery; a gene encoding double-stranded DNA-binding protein (DSB). The genes may be naturally occurring genes or may comprise one or more substitutions, insertions, or deletions. The genes may have at least 80%, 85%, 90%, 95%, 97%, 98%, 99%, or 100% similarity or identity to a naturally occurring gene from a φ29-like lytic phage from the Podoviridae family or a lytic phage from the Tectiviridea family. The genes may have at least 80%, 85%, 90%, 95%, 97%, 98%, 99%, or 100% similarity or identity to a naturally occurring gene from a lytic phage that is a member of the Tectiviridae family. The genes may have at least 80%, 85%, 90%, 95%, 97%, 98%, 99%, or 100% similarity or identity to a naturally occurring gene from any of phage PRD1, phage PR3, phage PR4, phage PR5, phage L17, phage PR722, or phage BCE1. Thus, a Podoviridae or Tectiviridae TP, Podoviridae or Tectiviridae SSB, or Podoviridae or Tectiviridae DSB as referred to herein encompasses the natural proteins and variants of said natural proteins that retain the appropriate DNA replication function.

As used herein, the term “SSB” refers to a protein from the orthogonal DNA replication machinery of a lytic phage of the Tectiviridea family or a lytic φ29-like virus of the Podoviridae family, wherein the SSB corresponds to the PRD1 phage protein P19. The SSB need not necessarily function as a single-stranded DNA-binding protein, for instance it may be capable of also serving as a double-stranded DNA-binding protein (e.g. it may have a dual function in the manner of PRD1 phage protein P19).

As used herein, the term “DSB” refers to a protein from the orthogonal DNA replication machinery of a lytic phage of the Tectiviridea family or a lytic φ29-like virus of the Podoviridae family, wherein the DSB corresponds to the PRD1 phage protein P12. The DSB need not necessarily function as a double-stranded DNA-binding protein, for instance it may be capable of also serving as a single-stranded DNA-binding protein (e.g. it may have a dual function in the manner of PRD1 phage protein P12).

Thus, the SSB and the DSB need only have the property of binding to DNA. They are identifiable by the skilled person as two of the four components of DNA replication machinery from the aforementioned phages and due to their correspondence with P19 and P12 of PRD1.

As discussed in Examples 17 and 18, the SSB is optional, particularly for embodiments where the orthogonal episome is delivered as a complex with the TP. As discussed in the Examples, the DSB is optional and not strictly necessary for maintenance of an orthogonal episome.

In examples, the cell of the first aspect comprises a gene encoding a Podoviridae or Tectiviridae TP. In particular examples, the cell of the first aspect comprises a gene encoding a Podoviridae or Tectiviridae TP, a gene encoding a Podoviridae or Tectiviridae SSB, and optionally a gene encoding a Podoviridae or Tectiviridae DSB. The cell also comprises a gene encoding an orthogonal DNA polymerase (ODNAP), which may be a Podoviridae or Tectiviridae DNA polymerase from a lytic phage as discussed herein. The ODNAP may be from or derived from any of phage PRD1, phage PR3, phage PR4, phage PR5, phage L17, phage PR722, or phage BCE1. The DNA polymerase may be a naturally occurring or engineered polymerase. For instance, the orthogonal DNA polymerase may be modified so as to increase the error rate or to render the DNA polymerase error prone. The orthogonal DNA polymerase may have a mutation rate of at least 10⁻⁹, 10⁻⁸, 10⁻⁷, 10⁻⁶, or 10⁻⁵ substitutions per base (s.p.b.). The gene encoding the DNA polymerase may be a naturally occurring gene or may comprise one or more substitutions, insertions, or deletions. The gene may have at least 80%, 85%, 90%, 95%, 97%, 98%, 99%, or 100% similarity or identity to a naturally occurring gene from a member of the Podoviridae or Tectiviridae family or a gene from any of phage PRD1, phage PR3, phage PR4, phage PR5, phage L 17, phage PR722, or phage BCE1. Thus, a Podoviridae or Tectiviridae DNA polymerase as referred to herein encompasses the natural polymerase and variants of said natural polymerase that retain the appropriate DNA replication function. The cell of the first aspect may comprise more than one ODNAP. For instance, the cell may comprise two ODNAPs with different mutation rates. The cell may comprise a wild-type ODNAP and a mutated error-prone ODNAP. The ODNAPs may be inducible under different conditions and so it is possible to alter the culture conditions to determine which ODNAP is active. Illustrative examples of inducible ODNAPs are provided in the experimental Examples section.

The SSB and optionally DSB may be encoded by a non-orthogonal episome. The SSB and optionally DSB may be encoded by a plasmid that can be maintained by the DNA polymerase endogenous to the cell. The SSB and optionally the DSB may be overexpressed. An overexpressed SSB or DSB may be under the control of an inducible promoter.

In particular examples, the cell of the first aspect does not comprise any of lytic or structural genes associated with a member of the Podoviridae or Tectiviridae family. For instance, the cell of the first aspect may comprise no Podoviridae or Tectiviridae family genes other than the TP and ODNAP genes and optionally inverted terminal repeats (ITRs). In another example, the cell of the first aspect may comprise no Podoviridae or Tectiviridae family genes other than the TP, SSB, optionally DSB, and ODNAP genes and optionally inverted terminal repeats (ITRs).

In a particular embodiment, the orthogonal DNA replication machinery is from or derived from PRD1 bacteriophage. Therefore, in a particular example, the cell of the first aspect comprises a gene encoding a PRD1 bacteriophage TP. In another particular example, the cell of the first aspect comprises a gene encoding a PRD1 bacteriophage TP, a gene encoding a PRD1 bacteriophage SSB, and optionally a gene encoding a PRD1 bacteriophage DSB. The genes may be naturally occurring genes or may comprise one or more substitutions, insertions, or deletions. The genes may have at least 80%, 85%, 90%, 95%, 97%, 98%, 99%, or 100% similarity or identity to a naturally occurring gene from a PRD1 bacteriophage. Thus, a PRD1 bacteriophage TP, PRD1 bacteriophage SSB, or PRD1 bacteriophage DSB as referred to herein encompasses the natural proteins and variants of said natural proteins that retain the appropriate DNA replication function. The bacterial cell also comprises an ODNAP, which may be a PRD1 bacteriophage DNA polymerase. The DNA polymerase may be a naturally occurring or engineered polymerase. For instance, the orthogonal DNA polymerase may be modified so as to increase the error rate or to render the DNA polymerase error prone. The orthogonal DNA polymerase may have a mutation rate of at least 10⁻⁹, 10⁻⁸, 10⁻⁷, 10⁻⁶, or 10⁻⁵ s.p.b. The gene encoding the DNA polymerase may be a naturally occurring gene or may comprise one or more substitutions, insertions, or deletions. The gene may have at least 80%, 85%, 90%, 95%, 97%, 98%, 99%, or 100% similarity or identity to a naturally occurring PRD1 bacteriophage ODNAP gene. Thus, a PRD1 bacteriophage DNA polymerase as referred to herein encompasses the natural polymerase and variants of said natural polymerase that retains the appropriate DNA replication function.

In particular examples, the cell of the first aspect does not comprise any lytic or structural genes derived from PRD1. PRD1 has a linear double stranded genome encodes at least 25 gene products from five annotated operons under the control of eight annotated promoters and terminators. The ends of the linear genome are composed of inverted terminal repeats that form the binding site for the TP and function as origins of replication. The early operons contain the genes responsible for replication of the PRD1 genome. The left early operon encodes the TP and the DNAP, and the right early operon encodes phage single strand and double strand DNA binding proteins (SSB and DSB, respectively). The central operons contain the genes that encode the remaining structural and lytic protein components of PRD1. In some embodiments, the cell of the first aspect comprises no PRD1 genes other than the two, three, or four genes mentioned in the preceding paragraphs and optionally ITRs.

An example of a sequence of a PRD1 TP is provided below.

(SEQ ID NO: 1)
MAKKKPVEKNGLVYKEFQKQVSNLKKAGLIPKTLDVRKVKPTKHYKGLV
SKYKDVATGGAKLAAIPNPAVIETLEARGESIIKKGGKAYLKARQQINQ
RGQIVNPFTVRVTKRGEVVRRYRKTTPEGKPVYITQRELPIKFENMEQW
LTELKAAGFQLQPGEQIYFTFNGNYSRRTYTSFDEAFNKFMTYDIIIDA
VAGKLKVEDEADLVKSVGFQRISGPEAKAYNRNRIVLPEMQFSQAAKKK
YKRRQKRGYGSKGV

An example of a sequence of a PRD1 ODNAP is provided below.

(SEQ ID NO: 2)
MPRRSRKKVEYKIAAFDFETDPFKHDRIPKPFSWGFYNGEIYKDYWGDD
CIEQFIYWLDTIEEPHVIYAHNGGKFDFLFLMKYFRGKLKIVNGRILEV
EHGIHKFRDSYAILPVPLAASDEKIEIDYGKMERETREQHKAEILEYLK
GDCVTLHKMVSLFIAEFGMRLTIGGTAMNELKQFHPYDPVRKGFDEAMR
PFYFGGRCQAFEKGIIEDDIKVYDVNSMYPHAMRNFRHPFSDEFYEANE
ITEETYFIEWEGENNGAVPVRTKTGLDFNQRSGIFHTSIHEWRAGIDTG
TIKPNRIIRTINFTETTTFGAFIDHFFSKRDAAKKAGDLFHNIFYKLIL
NSSYGKFAQNPENYKEWCITEGGIYLEGYDGEGCEVQEHLDYILWGRPA
EMFNYFNVAVAASITGAARSVLLRALAQAERPLYCDTDSIICRDLKNVP
LDAYQLGAWDLEATGDKIAIAGKKLYALYAGDNCVKIASKGASLVPRDI
GFLMPPDMEPKAAKKVAQQKAKNIGGEKILKVANGGVYDFVNDAPSFKL
NGNVQFIKRTIKGT

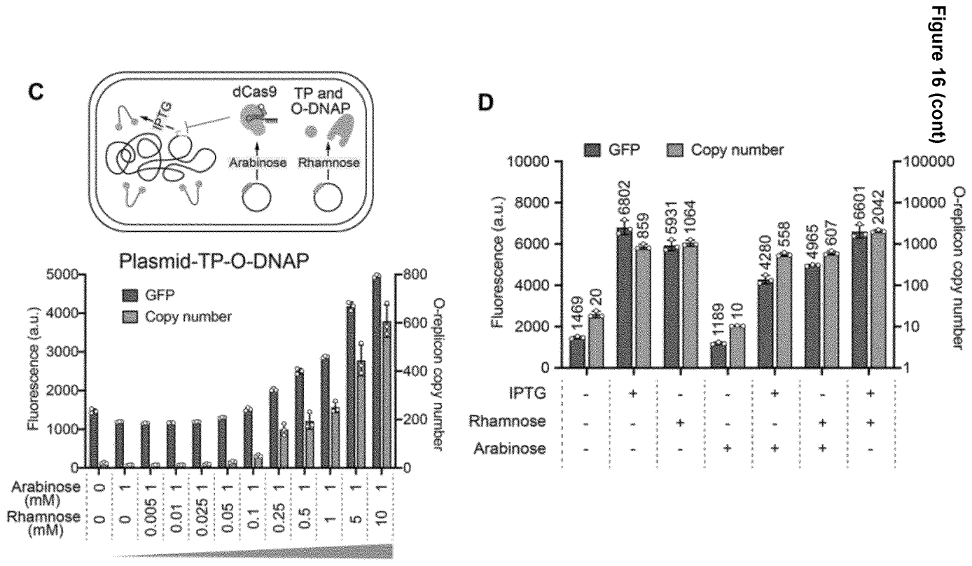

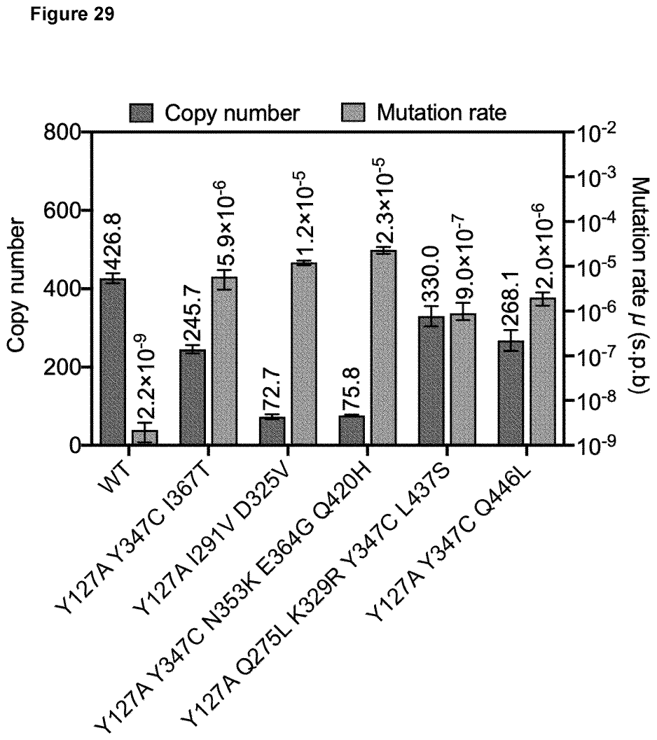

The ODNAP may be an error-prone ODNAP. For instance, the ODNAP may be mutated to disrupt the proofreading exonuclease activity. Examples of mutations that may be applied to SEQ ID NO: 2 include T20I, N71D, N71D & L341R, H70R, D17A, D76A, D17A & D76A, D17A & D76A & L341R, and/or Y127A (see FIG. 17). Other examples include Y347C, 1367T, 1291V, D325V, N353K, E364G, Q420H, Q275L, K329R, L437S, and Q446L (see FIG. 29). In the screen of Example 19, the mutations Y347C, D325V, K329R, and N353K were highly enriched, and thus any one or any combination of these mutations may be applied to SEQ ID NO: 2. Examples of sets of mutations that may be applied are: i) Y127A, Y347C, and 1367T; ii) Y127A, 1291V, and D325V; iii) Y127A, Y347C, N353K, E364G, and Q420H; iv) Y127A, Q275L, K329R, Y347C, and L437S; or v) Y127A, Y347C, and Q446L.

The ODNAP may be an error-prone polymerase according to SEQ ID NO: 2 and comprising any one, or any combination, of D17A, T201, H70R, N71D, D76A, Y127A, and L341R. The ODNAP may be an error-prone polymerase according to SEQ ID NO: 2 and comprising any one, or any combination, of D17A, T20I, H70R, N71D, D76A, Y127A, Q275L, 1291V, D325V, K329R, L341R, Y347C, N353K, E364G, 1367T, Q420H, L437S, and Q446L. The ODNAP may be an error-prone polymerase according to SEQ ID NO: 2 and comprising any one, or any combination, of Y347C, D325V, K329R, and N353K. Particularly exemplified ODNAPs include those comprising N71D or Y127A, those according to SEQ ID NO: 2 with N71D or Y127A, or those in FIG. 29.

An example of a sequence of a PRD1 DSB is provided below.

(SEQ ID NO: 3)
MEIVSKLTLKTIGAQPKPHSVKENTALASIYGRVRGKKVGQSTFGDFIK
FEGEFEGVNIATGEVFRSGALILPKVLESLLAGAVDGENTVDFAVEIWA
KPSEKGNTGYEYGVKPLIEPAASDELAALRNQVKAALPAPAAAGEAAAE
AKPAAKAKAKAEA

An example of a sequence of a PRD1 SSB is provided below.

(SEQ ID NO: 4)
MEKQTENTRPECPKAFYFVSIPGDFGQTPFASSLMYGSTALAAVYQVKG
AIRVVSEHFDLRFADNGFTPAGVTQAEWLGKLITETFGFRLELFL

In a particular example, the bacterial cell of the first aspect comprises one or more genes encoding proteins that are at least 80%, 85%, 90%, 95%, 97%, 98%, 99%, or 100% similar or identical to any one, two, or three of SEQ ID NOs: 1, 2, and 4. The cell may comprise a gene encoding a protein that is at least 80%, 85%, 90%, 95%, 97%, 98%, 99%, or 100% similar or identical to SEQ ID NO: 2, and comprising any of the mutations disclosed herein (e.g. as discussed above). The cell may also comprise a gene encoding a protein that is at least 80%, 85%, 90%, 95%, 97%, 98%, 99%, or 100% similar or identical to SEQ ID NO: 3. In another example, the bacterial cell of the first aspect comprises a gene encoding a protein that is at least 80%, 85%, 90%, 95%, 97%, 98%, 99%, or 100% similar or identical to any one, two, three, or four of SEQ ID NOs: 1, 2, 3, and 4.

In embodiments comprising only a TP and an ODNAP, the TP and/or the ODNAP may be encoded by a non-orthogonal episome within the cell. For instance, at least one or both of the TP and the ODNAP may be encoded by the genome of the cell. This provides a cell that contains the necessary machinery to replicate compatible orthogonal DNA. The cell may comprise one or more non-orthogonal plasmid, or other non-genomic episome, that encodes the TP and/or the ODNAP. Such plasmids may be referred to as “helper plasmids”. In some embodiments, the TP and the ODNAP are split between the genome of the cell and one or more non-orthogonal non-genomic episomes. The TP and the ODNAP may be encoded within a single operon. Thus, in an embodiment, the cell is a bacterial cell, which may be E. coli, wherein the bacterial cell genome comprises an operon encoding the TP and ODNAP.

One, two, or all three of the TP, SSB, and ODNAP may be encoded on a non-orthogonal episome within the cell (i.e. an episome that can be maintained by the endogenous DNA replication machinery of the host cell). One, two, or all three of the TP, SSB, and ODNAP may be encoded by the genome of the cell. The cell may be a bacterial cell comprising a genome encoding one, two, or all three of the TP, SSB, and ODNAP. One, two, or all three of the TP, SSB, and ODNAP may be encoded by a single operon. Thus, in an embodiment, the cell is a bacterial cell, which may be E. coli, wherein the bacterial cell genome comprises an operon encoding the TP, SSB, and ODNAP. In a particular embodiment, the operon comprises, from 5′ to 3′, the genes for TP, ODNAP, and SSB.

One, two, three, or all four of the TP, SSB, DSB, and ODNAP may be encoded on a non-orthogonal episome within the cell (i.e. an episome that can be maintained by the endogenous DNA replication machinery of the host cell). One, two, three, or all four of the TP, SSB, DSB, and ODNAP may be encoded by the genome of the cell.

The cell may be a bacterial cell comprising a genome encoding one, two, three, or all four of the TP, SSB, DSB, and ODNAP. One, two, three, or all four of the TP, SSB, DSB, and ODNAP may be encoded by a single operon.

Thus, in an embodiment, the cell is a bacterial cell, which may be E. coli, wherein the bacterial cell genome comprises an operon encoding the TP, SSB, DSB, and ODNAP. In a particular embodiment, the operon comprises, from 5′ to 3′, the genes for TP, ODNAP, DSB, and SSB.

The genes may be codon optimised for the host cell. As an example, the cell may comprise a gene cluster encoding a TP, an ODNAP, optionally a DSB, and optionally an SSB, each of which has been optimised for the host.

A purely illustrative example of a PRD1 gene cluster encoding a TP, ODNAP, DSB, and an SSB is provided below. It encodes a TP (557 . . . 1336), a DNA polymerase (1340 . . . 3001), a DSB (3064 . . . 3564), and a SSB (3619 . . . 3903).

(SEQ ID NO: 5)
taccccaaattgcgcccctgcaatttgctgacggtttaacggaaggcggaaacatggcgaagaaaaaaccagtaga
aaaaaatgggcttgtttataaagagtttcaaaaacaagtttcaaatttgaagaaagccggactaatccctaaaacc
cttgacgtgcgaaaagtcaagccaacaaaacactataaaggattggtaagcaaatataaagacgttgcaacagggg
gcgctaaacttgcagcaatccctaaccccgccgttattgaaacgcttgaagcgcggggcgaatccatcattaagaa
aggcggcaaggcgtatctgaaagcccgccagcaaataaaccagcgcgggcaaattgtaaacccctttacggttcgc
gtaaccaaacgcggcgaagtggtgcgccgctaccgcaagactaccccggaaggcaagcccgtttatatcacgcaac
gggaattgcctattaagtttgaaaatatggaacagtggcttactgaattaaaggccgctggttttcaattgcaacc
gggcgaacaaatctatttcacttttaacggcaactattcccgccgtacctatacgtcatttgatgaagcgttcaat
aaatttatgacgtatgacattattattgatgcggtggccggaaaattaaaagtagaagatgaagccgatttagtta
agtcggtaggctttcaacgtatcagcggccccgaagccaaggcgtataaccgtaaccgtattgtattgcctgaaat
gcaatttagccaagcggctaaaaagaaatacaagcgccgtcaaaaacgcggctatggcagcaagggggtttaagat
atgccgcgccgttcccgtaaaaaggtggaatataaaattgccgcctttgactttgaaactgaccctttcaagcatg
accgaatccctaaaccgttttcatggggtttttataatggcgaaatttataaagactattggggcgatgattgcat
agaacagtttatttactggctggataccatagaagaaccgcacgttatatacgctcataacggcggcaagtttgat
tttctttttctcatgaaatactttcgcgggaaattgaaaatagttaatgggcgtattttggaagtagaacacggca
tccataaattccgcgatagttatgcaatcctgccggtgccgcttgctgccagcgatgaaaagatagaaattgatta
tggcaagatggaaagggaaacacgcgaacagcacaaggcggaaattttagaatacctgaaaggcgattgtgtaacc
ctgcataaaatggtttctttatttattgctgaatttggaatgcgcctaaccataggcggtacggcaatgaatgaat
taaaacagttccacccttatgaccctgtgcgcaaaggctttgatgaagccatgcgccccttttattttggcggaag
gtgccaagcattcgagaaaggaataattgaagatgatataaaagtttatgatgttaatagtatgtacccccatgct
atgcgaaatttccgccatcctttcagcgatgaattttatgaagccaatgaaataacagaagaaacttattttattg
aatgggaaggcgagaataacggcgcggtgcctgttaggactaaaacaggtttagactttaatcagcgtagcggcat
tttccatacgtcaatccatgaatggcgggcgggtattgataccggcacgattaaacctaatcggattataaggaca
atcaattttactgaaacaaccactttcggcgcattcattgaccatttctttagcaagcgtgacgctgccaaaaagg
cgggtgatttattccacaatattttttacaaactgattttaaatagcagttatgggaagtttgcacaaaaccccga
aaattataaagagtggtgcataacggaaggcggcatttatttagaaggctatgacggcgaagggtgcgaagtacag
gaacatttagactatattttatggggtaggcccgctgaaatgtttaattattttaacgtggcagtggcggcaagta
ttacaggcgcggcccgttccgttttattgcgagcattggcgcaagcggaaaggccgctttattgcgacactgattc
tattatctgccgtgatttaaaaaatgttccgcttgacgcataccagctaggcgcgtgggatttggaagcaaccggc
gataaaatagcgattgccggtaaaaaattatatgcgctttacgctggtgataattgcgttaaaattgcaagtaagg
gggctagtctggttccgcgtgatattgggtttttaatgcccccggatatggaaccgaaagccgccaaaaaggtagc
gcaacaaaaggctaaaaatattggtggcgagaaaattttaaaggtggctaatggcggcgtgtatgattttgtaaat
gatgccccgtcatttaagctaaatggcaacgtgcaatttatcaagcgcacaatcaaaggaacataaatcgaaaggg
caaacacaaaacccccgccgataacttccacttaactttaaaggtaactatcatggaaatcgtaagcaagctgact
ctgaaaaccattggcgcacaaccgaagccgcatagcgtaaaagaaaataccgcgctggcttccatctatggccgcg
ttcgcggtaagaaagttggtcaatccacctttggcgacttcatcaagtttgaaggtgaatttgaaggcgtgaatat
cgccactggtgaagtgttccgttccggtgcgctgattctgccgaaggtactggaaagcctgcttgccggtgccgtg
gatggtgaaaacacggttgattttgcggttgaaatttgggccaagccttccgaaaagggcaacactggttatgaat
atggtgtcaagccgctgattgaacccgccgcatcggatgaactggccgcgcttcgcaatcaggttaaggccgcgct
gcctgcccctgccgctgccggtgaagccgctgccgaagccaagcccgccgccaaggccaaggccaaggccgaagcc
taaacagcgcaccacggccccggctgataccagcgcggggccattctttacccttcaaattcaaaggtaacaaaca
tggaaaagcaaaccgaaaatacccgccctgaatgcccgaaggcattttattttgtatccattcccggcgactttgg
gcaaaccccttttgcttcatcgcttatgtatggtagcaccgcgcttgccgctgtttatcaagtcaagggcgctatc
cgcgtagtaagcgaacattttgatttgcggtttgctgataacggttttacgcctgctggcgttacgcaagctgaat
ggcttggcaagctgattactgaaactttcggctttcgcttggaactgtttctttaaaccagttttttatttcttga
atttgaaggccgcatgattcccttgcggccttttcttttgcgtatgcttccaccaaatcccgccacgttataaacg
gataaacaggcgcggcgcattcagttagcaggcttgacgg

In an example, a bacterial cell of the first aspect comprises an operon comprising a nucleic acid sequence that is at least 80%, 85%, 90%, 95%, 97%, 98%, 99%, or 100% identical to SEQ ID NO: 5.

The operon may be designed according to the methods disclosed in WO2023285596 or Dunkelmann et al. (A 68-codon genetic code to incorporate four distinct non-canonical amino acids enabled by automated orthogonal mRNA design. Nat. Chem. 13, 1110-1117 (2021). https://doi.org/10.1038/s41557-021-00764-5), each of which is herein incorporated by reference. These documents disclose methods that enable the design of suitable synthetic ribosome binding sites. For instance, WO2023285596 discloses a method of designing an operon comprising at least two exogenous open reading frames for expression in a host cell. Thus, one, two, three, or all four of the TP, SSB, DSB, and ODNAP may be encoded by a single operon and the operon may be have been optimised according to the techniques disclosed in the aforementioned documents. Such techniques may not be relevant where the phage is naturally compatible with the host cell.

The operon may comprise an inducible promoter operably linked to the genes; illustrative examples include the Parabinose promoter and the PIPTG promoter, or variants thereof. The operon may comprise a constitutive endogenous promoter operably linked to the genes; an illustrative example is the PdnaKJ promoter or a variant thereof. The operon may comprise a LexA-controlled promoter operably linked to the genes; illustrative examples include the PrecN promoter and the PuvrB promoter, or variants thereof. The operon may comprise a self-regulating promoter; an illustrative example is the PpN15 or a variant thereof. The operon may comprise an Anderson promoter; an illustrative example is the PJ23114 promoter or a variant thereof.

A purely illustrative example of an operon encoding a TP, ODNAP, DSB, and an SSB is provided below. The below operon includes the PRD1 gene cluster of SEQ ID NO: 5 (504 . . . 4039). It also comprises a region encoding a beta-D-galactosidase (1 . . . 406), a promoter that is a hybrid between the trp and lac UV5 promoters (449 . . . 477), a lac operator (485 . . . 501), and a DNA-binding transcriptional repressor (lacI). As the skilled person would 50 appreciate, these features are exemplary and not limiting. In this example, the lac repressor binds to the lac operator to inhibit transcription in E. coli. This inhibition can be relieved by adding lactose or isopropyl-beta-D-thiogalactopyranoside (IPTG).

(SEQ ID NO: 6)
aaacgactgtcctggccgtaaccgacccagcgcccgttgcaccacagatgaaacgccgagttaacgccatcaaaaa
taattcgcgtctggccttcctgtagccagctttcatcaacattaaatgtgagcgagtaacaacccgtcggattctc
cgtgggaacaaacggcggattgaccgtaatgggataggtcacgttggtgtagatgggcgcatcgtaaccgtgcatc
tgccagtttgaggggacgacgacagtatcggcctcaggaagatcgcactccagccagctttccggcaccgcttctg
gtgccggaaaccaggcaaagcgccattcgccattcaggctgcgcaactgttgggaagggcgatcggtgcgggccac
gacggccagtgaatccgtaatcatggccgacatcataacggttctggcaaatattctgaaatgagctgttgacaat
taatcatcggctcgtataatgtgtggaattgtgagcggataacaatttaccccaaattgcgcccctgcaatttgct
gacggtttaacggaaggcggaaacatggcgaagaaaaaaccagtagaaaaaaatgggcttgtttataaagagtttc
aaaaacaagtttcaaatttgaagaaagccggactaatccctaaaacccttgacgtgcgaaaagtcaagccaacaaa
acactataaaggattggtaagcaaatataaagacgttgcaacagggggcgctaaacttgcagcaatccctaacccc
gccgttattgaaacgcttgaagcgcggggcgaatccatcattaagaaaggcggcaaggcgtatctgaaagcccgcc
agcaaataaaccagcgcgggcaaattgtaaacccctttacggttcgcgtaaccaaacgcggcgaagtggtgcgccg
ctaccgcaagactaccccggaaggcaagcccgtttatatcacgcaacgggaattgcctattaagtttgaaaatatg
gaacagtggcttactgaattaaaggccgctggttttcaattgcaaccgggcgaacaaatctatttcacttttaacg
gcaactattcccgccgtacctatacgtcatttgatgaagcgttcaataaatttatgacgtatgacattattattga
tgcggtggccggaaaattaaaagtagaagatgaagccgatttagttaagtcggtaggctttcaacgtatcagcggc
cccgaagccaaggcgtataaccgtaaccgtattgtattgcctgaaatgcaatttagccaagcggctaaaaagaaat
acaagcgccgtcaaaaacgcggctatggcagcaagggggtttaagatatgccgcgccgttcccgtaaaaaggtgga
atataaaattgccgcctttgactttgaaactgaccctttcaagcatgaccgaatccctaaaccgttttcatggggt
ttttataatggcgaaatttataaagactattggggcgatgattgcatagaacagtttatttactggctggatacca
tagaagaaccgcacgttatatacgctcataacggcggcaagtttgattttctttttctcatgaaatactttcgcgg
gaaattgaaaatagttaatgggcgtattttggaagtagaacacggcatccataaattccgcgatagttatgcaatc
ctgccggtgccgcttgctgccagcgatgaaaagatagaaattgattatggcaagatggaaagggaaacacgcgaac
agcacaaggcggaaattttagaatacctgaaaggcgattgtgtaaccctgcataaaatggtttctttatttattgc
tgaatttggaatgcgcctaaccataggcggtacggcaatgaatgaattaaaacagttccacccttatgaccctgtg
cgcaaaggctttgatgaagccatgcgccccttttattttggcggaaggtgccaagcattcgagaaaggaataattg
aagatgatataaaagtttatgatgttaatagtatgtacccccatgctatgcgaaatttccgccatcctttcagcga
tgaattttatgaagccaatgaaataacagaagaaacttattttattgaatgggaaggcgagaataacggcgcggtg
cctgttaggactaaaacaggtttagactttaatcagcgtagcggcattttccatacgtcaatccatgaatggcggg
cgggtattgataccggcacgattaaacctaatcggattataaggacaatcaattttactgaaacaaccactttcgg
cgcattcattgaccatttctttagcaagcgtgacgctgccaaaaaggcgggtgatttattccacaatattttttac
aaactgattttaaatagcagttatgggaagtttgcacaaaaccccgaaaattataaagagtggtgcataacggaag
gcggcatttatttagaaggctatgacggcgaagggtgcgaagtacaggaacatttagactatattttatggggtag
gcccgctgaaatgtttaattattttaacgtggcagtggcggcaagtattacaggcgcggcccgttccgttttattg
cgagcattggcgcaagcggaaaggccgctttattgcgacactgattctattatctgccgtgatttaaaaaatgttc
cgcttgacgcataccagctaggcgcgtgggatttggaagcaaccggcgataaaatagcgattgccggtaaaaaatt
atatgcgctttacgctggtgataattgcgttaaaattgcaagtaagggggctagtctggttccgcgtgatattggg
tttttaatgcccccggatatggaaccgaaagccgccaaaaaggtagcgcaacaaaaggctaaaaatattggtggcg
agaaaattttaaaggtggctaatggcggcgtgtatgattttgtaaatgatgccccgtcatttaagctaaatggcaa
cgtgcaatttatcaagcgcacaatcaaaggaacataaatcgaaagggcaaacacaaaacccccgccgataacttcc
acttaactttaaaggtaactatcatggaaatcgtaagcaagctgactctgaaaaccattggcgcacaaccgaagcc
gcatagcgtaaaagaaaataccgcgctggcttccatctatggccgcgttcgcggtaagaaagttggtcaatccacc
tttggcgacttcatcaagtttgaaggtgaatttgaaggcgtgaatatcgccactggtgaagtgttccgttccggtg
cgctgattctgccgaaggtactggaaagcctgcttgccggtgccgtggatggtgaaaacacggttgattttgcggt
tgaaatttgggccaagccttccgaaaagggcaacactggttatgaatatggtgtcaagccgctgattgaacccgcc
gcatcggatgaactggccgcgcttcgcaatcaggttaaggccgcgctgcctgcccctgccgctgccggtgaagccg
ctgccgaagccaagcccgccgccaaggccaaggccaaggccgaagcctaaacagcgcaccacggccccggctgata
ccagcgcggggccattctttacccttcaaattcaaaggtaacaaacatggaaaagcaaaccgaaaatacccgccct
gaatgcccgaaggcattttattttgtatccattcccggcgactttgggcaaaccccttttgcttcatcgcttatgt
atggtagcaccgcgcttgccgctgtttatcaagtcaagggcgctatccgcgtagtaagcgaacattttgatttgcg
gtttgctgataacggttttacgcctgctggcgttacgcaagctgaatggcttggcaagctgattactgaaactttc
ggctttcgcttggaactgtttctttaaaccagttttttatttcttgaatttgaaggccgcatgattcccttgcggc
cttttcttttgcgtatgcttccaccaaatcccgccacgttataaacggataaacaggcgcggcgcattcagttagc
aggcttgacgggggagaccagaaacaaaaaaacacccgttagggtgttttttcgaaaaggctggtcattgtccact
ctctaaacgagatacttgacgagcgagttgcatcagagaatctgccaatgcacgaggagaggcggtttgcgtattg
ggcgccagggtggtttttcttttcaccagtgagacgggcaacagctgattgcccttcaccgcctggccctgagaga
gttgcagcaagcggtccacgctggtttgccccagcaggcgaaaatcctgtttgatggtggttaacggcgggatata
acatgagctgtcttcggtatcgtcgtatcccactaccgagatatccgcaccaacgcgcagcccggactcggtaatg
gcgcgcattgcgcccagcgccatctgatcgttggcaaccagcatcgcagtgggaacgatgccctcattcagcattt
gcatggtttgttgaaaaccggacatggcactccagtcgccttcccgttccgctatcggctgaatttgattgcgagt
gagatatttatgccagccagccagacgcagacgcgccgagacagaacttaatgggcccgctaacagcgcgatttgc
tggtgacccaatgcgaccagatgctccacgcccagtcgcgtaccgtcttcatgggagaaaataatactgttgatgg
gtgtctggtcagagacatcaagaaataacgccggaacattagtgcaggcagcttccacagcaatggcatcctggtc
atccagcg

There is provided herein a cell, such as a bacterial cell that is optionally an E. coli cell, comprising a gene encoding a Tectiviridae TP and a gene encoding an ODNAP. There is provided herein a cell, such as a bacterial cell that is optionally an E. coli cell, expressing a Tectiviridae TP and an ODNAP. For example, the Tectiviridae family member may be any one of phage PRD1, phage PR3, phage PR4, phage PR5, phage L17, phage PR722, and phage BCE1. In some embodiments, the genes are recombinant genes and are present on one or more non-orthogonal (i.e. endogenously replicated) episomes.

There is provided herein a cell, such as a bacterial cell that is optionally an E. coli cell, comprising a gene encoding a PRD1 bacteriophage TP and a gene encoding an ODNAP. There is provided herein a cell, such as a bacterial cell that is optionally an E. coli cell, expressing a PRD1 bacteriophage TP and an ODNAP. The genes may be any as described herein. In some embodiments, the genes are recombinant genes and are present on one or more non-orthogonal (i.e. endogenously replicated) episomes.

There is provided herein a cell, such as a bacterial cell that is optionally an E. coli cell, comprising a gene encoding a Tectiviridae TP, a gene encoding an ODNAP, a gene encoding a Tectiviridae SSB, and optionally a gene encoding a Tectiviridae DSB. There is provided herein a cell, such as a bacterial cell that is optionally an E. coli cell, expressing a Tectiviridae TP, an ODNAP, a Tectiviridae SSB, and optionally a Tectiviridae DSB. For example, the Tectiviridae family member may be any one of phage PRD1, phage PR3, phage PR4, phage PR5, phage L17, phage PR722, and phage BCE1. In some embodiments, the genes are recombinant genes and are present on one or more non-orthogonal (i.e. endogenously replicated) episomes.

There is provided herein a cell, such as a bacterial cell that is optionally an E. coli cell, comprising a gene encoding a PRD1 bacteriophage TP, a gene encoding an ODNAP, a gene encoding a PRD1 bacteriophage SSB, and optionally a gene encoding a PRD1 bacteriophage DSB. There is provided herein a cell, such as a bacterial cell that is optionally an E. coli cell, expressing a PRD1 bacteriophage TP, an ODNAP, a PRD1 bacteriophage SSB, and optionally a PRD1 bacteriophage DSB. The genes may be any as described herein. In some embodiments, the genes are recombinant genes and are present on one or more non-orthogonal (i.e. endogenously replicated) episomes.

The inventors demonstrate in Examples 17 and 18 that the orthogonal episome, once established (for instance, in cells comprising a TP, ODNAP, SSB, and optionally a DSB) can be extracted in a manner that maintains the bound proteins. This complex can then be transferred to new cells. The new cells only require the TP and the ODNAP to maintain the orthogonal episome and they do not require the SSB or the DSB. Thus, in an embodiment the cell comprising a gene encoding a Podoviridae or Tectiviridae TP and a gene encoding an ODNAP and does not comprise (or does not express or is not required to express) a gene encoding a Podoviridae or Tectiviridae SSB or DSB. In another embodiment the cell comprising a gene encoding a PRD1 TP and a gene encoding an ODNAP does not comprise (or does not express or is not required to express) a gene encoding a PRD1 SSB or PRD1 DSB.

The TP and/or the ODNAP may be encoded by a non-orthogonal episome within the cell. For instance, at least one or both of the TP and the ODNAP may be encoded by the genome of the cell. This provides a cell that contains the necessary machinery to replicate compatible orthogonal DNA. The cell may comprise one or more non-orthogonal plasmid, or other non-genomic episome, that encodes the TP and/or the ODNAP. In some embodiments, the TP and the ODNAP are split between the genome of the cell and one or more non-orthogonal non-genomic episomes. The TP and the ODNAP may be encoded within a single operon. The genes encoding the TP and the ODNAP may be referred to as recombinant genes. These genes are derived from phage but are encoded on non-orthogonal episomes within the cell, i.e. episomes that cannot be replicated by the wild-type phage DNA polymerase.

The TP and the ODNAP may be any as described herein. For example, in some embodiments, the TP and/or the ODNAP are from or derived from a Tectiviridae family member. In an embodiment, the TP and/or the ODNAP are from or derived from any of phage PRD1, phage PR3, phage PR4, phage PR5, phage L17, phage PR722, and phage BCE1. In a particular embodiment, the TP and the ODNAP are from phage PRD1. The genes may have at least 80%, 85%, 90%, 95%, 97%, 98%, 99%, or 100% similarity or identity to a naturally occurring gene. The TP and/or ODNAP genes may have at least 80%, 85%, 90%, 95%, 97%, 98%, 99%, or 100% similarity or identity to a naturally occurring gene from a PRD1 bacteriophage. The DNA polymerase may be a naturally occurring or engineered polymerase, as discussed for the first aspect. For instance, the orthogonal DNA polymerase may be modified to increase the error rate or to render the DNA polymerase error prone. The orthogonal DNA polymerase may have a mutation rate of at least 10⁻⁹, 10⁻⁸, 10⁻⁷, 10⁻⁶, or 10⁻⁵ s.p.b. The cell may comprise one or more genes encoding proteins that are at least 80%, 85%, 90%, 95%, 97%, 98%, 99%, or 100% similar or identical to SEQ ID NO: 1 and/or SEQ ID NO: 2. The cell may comprise a gene encoding a protein that is at least 80%, 85%, 90%, 95%, 97%, 98%, 99%, or 100% similar or identical to SEQ ID NO: 2, and comprising any of the mutations disclosed herein.

In particular examples, the cells do not comprise any of lytic or structural genes associated with a member of the Podoviridae or Tectiviridae family, as discussed above. For instance, the cell of these embodiments may comprise no Podoviridae or Tectiviridae family genes other than the TP and ODNAP genes and optionally inverted terminal repeats (ITRs). In particular examples, the cells of these embodiments do not comprise any lytic or structural genes derived from PRD1. In some embodiments, the cell of these embodiments comprise no PRD1 genes other than the TP and ODNAP and optionally ITRs.

Any of the cells of the first aspect may comprise a gene encoding Gam protein from the lambda phage. The cell of the first aspect may be a bacterial cell, such as an E. coli cell, that comprises a gene encoding Gam protein from the lambda phage. The cell of the first aspect may be a bacterial cell, such as an E. coli cell, that expresses Gam protein from the lambda phage. The Gam protein may be a wild-type protein or derivative that protects linear double-stranded DNA from degradation. The Gam protein may be a wild-type protein or derivative that inhibits host nucleases RecBCD and/or sbcCD.

The Gam protein may be encoded on a non-orthogonal episome, such as the cell's genome or a plasmid. The Gam protein may be overexpressed. An overexpressed Gam protein may be under the control of an inducible promoter.

The cell of the first aspect may overexpress Gam, SSB, and DSB. The cell of the first aspect may overexpress Gam, PRD1 SSB, and PRD1 DSB.

The cell of the first aspect is capable of replicating orthogonal DNA that is compatible with the orthogonal DNA replication machinery. As such, one or more episomes compatible with the orthogonal DNA replication machinery may be present within the cell. Such episomes may be referred to as “orthogonal episomes”.

The cell of the first aspect may comprise at least one, at least two, or at least three orthogonal episomes. The cell of the first aspect may comprise only one, only two, or only three orthogonal episomes.

Thus, in an embodiment, the cell of the first aspect comprises an episome that is capable of being replicated by the orthogonal replication machinery. In embodiments where the cell comprises DNA replication machinery from a bacteriophage of the Podoviridae or Tectiviridae family, the episome comprises a 5′ inverted terminal repeat (ITR) and a 3′ ITR, wherein the 5′ ITR and the 3′ ITR are compatible with Podoviridae or Tectiviridae DNA replication machinery, respectively. In embodiments where the cell comprises DNA replication machinery from a PRD1 bacteriophage, the episome comprises a 5′ ITR and a 3′ ITR, wherein the 5′ ITR and the 3′ ITR are compatible with PRD1 bacteriophage DNA replication machinery. The episome may be a linear plasmid. The orthogonal episome may not encode one, two, three, or all four of a TP, a DNA polymerase, an SSB, and a DSB. In particular embodiments, the episome does not comprise sequences of the bacteriophage genome other than the ITRs.

The orthogonal episome may comprise a sequence-of-interest. For instance, a gene to which it is desirable to apply continuous directed evolution techniques. The gene may encode a protein or polypeptide for which particular functionality, or an increase or decrease in functionality, is desired. The sequence-of-interest may have a function in its own right. For instance, the sequence-of-interest may be a promoter.

Purely illustrative examples that feature in the experimental Examples section include an antibiotic resistance gene, a fluorescent protein, and a promoter. However, the invention is not limited to these illustrations and, as will be apparent to the skilled person, the invention is compatible with any sequence-of-interest for which a change in functionality, efficacy, or similar may be brought about by mutations.

In a particular embodiment, the episome is a linear plasmid comprising: a 5′ ITR, a sequence-of-interest, and a 3′ ITR, wherein the 5′ ITR and the 3′ ITR are compatible with PRD1 bacteriophage DNA replication machinery.

The linear plasmid may lack 5′ phosphorylation. For instance, the linear plasmid may have been generated using non-phosphorylated primers. Linear plasmids lacking 5′ phosphorylation are associated with an improvement, as shown in the experimental Examples section.

The orthogonal episome may comprise a marker. For instance, a selectable marker. The marker may be a positive marker such that cells comprising the orthogonal episome may be identified or selected, or so that selection pressure can be applied to a population of cells to increase the retention of the orthogonal episome.

In an example, a 5′ ITR compatible with PRD1 bacteriophage DNA replication machinery is as below.

(SEQ ID NO: 7)
GGGGATACGTGCCCCTCCCCACCTACCCGCGCCCCTAACATTTTTATTT
CCGTCTGTCAATACCCCCTGCATCCGATAGGCCCGAACTATCACAAACG
GAAAAGCGATAGCCCAAAACACTAAGCCCCTTT

The 5′ ITR may be truncated. In an example, the 5′ ITR may comprise at least the first 18, 19, 20, 21, 22, 23, 24, 25, 30, 40, 50, 60, 70, 100, or 110 bases of SEQ ID NO: 7.

In an example, a truncated 5′ ITR compatible with PRD1 bacteriophage DNA replication machinery is as below.

(SEQ ID NO: 10)
GGGGATACGTGCCCCTCC

The 5′ ITR may be or may comprise SEQ ID NO: 10. The 5′ ITR may comprise from 18 to 131 base pairs of SEQ ID NO: 7 and comprise SEQ ID NO: 10. The 5′ ITR may be a truncation of SEQ ID NO: 7 that is from 18 to 130, 18 to 110, 18 to 60, or 18 to 40 base pairs in length and is truncated from the 3′ end of SEQ ID NO: 7 (i.e. includes SEQ ID NO: 10).

In an example, a 3′ ITR compatible with PRD1 bacteriophage DNA replication machinery is as below.

(SEQ ID NO: 8)
AAATAAATCAAAATTTTATTCCTATCGCTTTTCCGTTTGTGATAGTTCG
GGCCTATCGGATGCAGGGGGTATTGACAGACGGAAATAAAAATGTTAGG
GGCGCGGGTAGGTGGGGAGGGGCACGTATCCCC

The 3′ ITR may be truncated. In an example, the 3′ ITR may comprise at least the last 18, 19, 20, 21, 22, 23, 24, 25, 30, 40, 50, 60, 70, 100, or 110 bases of SEQ ID NO: 8.

In an example, a truncated 3′ ITR compatible with PRD1 bacteriophage DNA replication machinery is as below.

(SEQ ID NO: 11)
GGAGGGGCACGTATCCCC

The 3′ ITR may be or may comprise SEQ ID NO: 11. The 3′ ITR may comprise from 18 to 131 base pairs of SEQ ID NO: 8 and comprise SEQ ID NO: 11. The 3′ ITR may be a truncation of SEQ ID NO: 8 that is from 18 to 130, 18 to 110, 18 to 60, or 18 to 40 base pairs in length and is truncated from the 5′ end of SEQ ID NO: 8 (i.e. includes SEQ ID NO: 11).

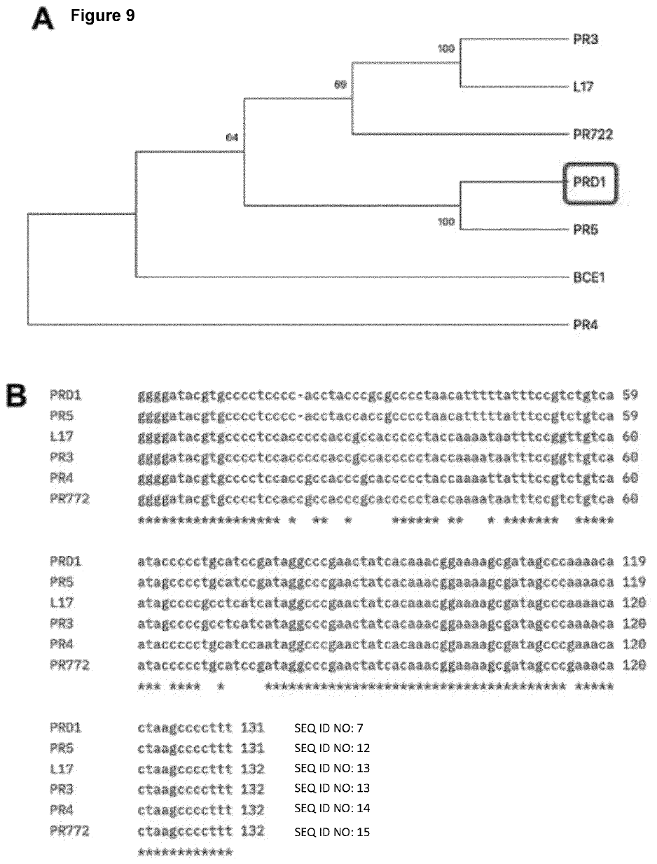

The inventors have found that the 18-base-pair ITRs retain functionality and thus may be used in the linear plasmids of the invention (see the Examples section). The inventors have also found that the first 18 base pairs are conserved amongst Tectiviridae family phages (see FIG. 9). Thus, ITRs according to SEQ ID NOs: 10 and 11 may be used in conjunction with non-PRD1 ODNAPs that are derived from members of the Tectiviridae family.

In a particular example, the linear plasmid comprises a 5′ ITR that is at least 80%, 85%, 90%, 95%, 97%, 98%, 99%, or 100% identical to SEQ ID NO: 7 or 10 and/or a 3′ ITR that is at least 80%, 85%, 90%, 95%, 97%, 98%, 99%, or 100% identical to SEQ ID NO: 8 or 11. The linear plasmid may comprise a truncation of SEQ ID NO: 7 and/or SEQ ID NO: 8 that is capable of being replicated by the orthogonal DNA replication machinery. In a particular example, the linear plasmid comprises a 5′ ITR that is at least 80%, 85%, 90%, 95%, 97%, 98%, 99%, or 100% identical to a truncation of SEQ ID NO: 7 and/or a 3′ ITR that is at least 80%, 85%, 90%, 95%, 97%, 98%, 99%, or 100% identical to a truncation of SEQ ID NO: 8, and wherein the linear plasmid is capable of being replicated by the orthogonal DNA replication machinery.

Other examples of 5′ ITRs that are suitable for use with the invention are displayed in FIG. 9B. Each of the 5′ ITRs are listed in the figure as relevant to a phage (e.g. PR5, L17, etc) and so may be relevant to a linear plasmid for maintenance in a cell comprising orthogonal replication machinery derived from said phage. The linear plasmid may also include the corresponding 3′ ITR. FIG. 9 shows that the ITRs are conserved and so may be compatible more broadly, for instance ITRs according to SEQ ID NO: 10 and SEQ ID NO: 11 would be compatible with replication machinery derived from each of PRD1, PR5, L17, PR3, PR4, and PR772 because these sequences are conserved.

A purely illustrative example of linear plasmid is provided below. This linear plasmid comprises the 5′ and 3′ ITRs of SEQ ID NOs: 7 and 8. It also comprises a Kan^{R} gene that confers resistance to kanamycin in bacteria (151 . . . 1187 wherein the protein coding region is 272 . . . 1087), a transcription terminator for bacteriophage T7 RNA polymerase (1188 . . . 1235), a sequence encoding sfGPF (1266 . . . 2009), a 6×His affinity tag, and a promoter that is a hybrid between the trp and lac UV5 promoters (2127 . . . 2257). As the skilled person would appreciate, these features are exemplary and not limiting.

(SEQ ID NO: 9)
ggggatacgtgcccctccccacctacccgcgcccctaacatttttattt
ccgtctgtcaataccccctgcatccgataggcccgaactatcacaaacg
gaaaagcgatagcccaaaacactaagccccttttcatcatttcatttgt
aagcagctgcagggaaagccacgttgtgtctcaaaatctctgatgttac
attgcacaagataaaaatatatcatcatgaacaataaaactgtctgctt
acataaacagtaatacaaggggtgttatgagccatattcaacgggaaac
gtcttgctcgaggccgcgattaaattccaacatggatgctgatttatat
gggtataaatgggctcgcgataatgtcgggcaatcaggtgcgacaatct
atcgattgtatgggaagcccgatgcgccagagttgtttctgaaacatgg
caaaggtagcgttgccaatgatgttacagatgagatggtcagactaaac
tggctgacggaatttatgcctcttccgaccatcaagcattttatccgta
ctcctgatgatgcatggttactcaccactgcgatccccgggaaaacagc
attccaggtattagaagaatatcctgattcaggtgaaaatattgttgat
gcgctggcagtgttcctgcgccggttgcattcgattcctgtttgtaatt
gtccttttaacagcgatcgcgtatttcgtctcgctcaggcgcaatcacg
aatgaataacggtttggttgatgcgagtgattttgatgacgagcgtaat
ggctggcctgttgaacaagtctggaaagaaatgcataagcttttgccat
tctcaccggattcagtcgtcactcatggtgatttctcacttgataacct
tatttttgacgaggggaaattaataggttgtattgatgttggacgagtc
ggaatcgcagaccgataccaggatcttgccatcctatggaactgcctcg
gtgagttttctccttcattacagaaacggctttttcaaaaatatggtat
tgataatcctgatatgaataaattgcagtttcatttgatgctcgatgag
tttttctaatcagaattggttaattggttgctgcagtgggttgatgata
ccgctgccttactgggtgcattagccagtctgaatgacctgtcacggga
taatccgaagtcaaaaaacccctcaagacccgtttagaggccccaaggg
gttatgctagttattgctcagcggtggcagcagccaactcTTAATGGTG
ATGATGATGGTGGCTGCCTTTATACAGTTCATCCATACCGTGGGTAATG
CCCGCGGCGGTAACAAATTCCAGCAGCACCATATGATCACGTTTTTCAT
TCGGATCTTTGCTCAGAACGCTCTGGGTGCTCAGATAATGATTATCCGG
CAGCAGCACCGGGCCATCACCAATCGGGGTATTCTGCTGataATGATCC
GCCAGCTGCACGCTACCATCTTCCACGTTGTGACGGATTTTAAAGTTCG
CTTTGATGCCATTTTTCTGTTTATCGGCGGTAATATACACGTTATGGCT
GTTGAAATTATATTCCAGTTTATGACCCAGAATGTTGCCatcTTCTTTA
AAATCAATACCTTTCAGTTCAATGCGGTTCACCAGGGTATCGCCTTCAA
ATTTAACTTCCGCACGGGTTTTATAGGTGCCATCATCTTTGAAGCTAAT
GGTACGTTCCTGCACATAGCCTTCCGGCATCGCGCTTTTAAAGAAATCA
TGGCGTTTCATATGATCCGGATAGCGGCTAAAGCACTGAACGCCATAat
gCAGGGTGGTCACCAGGGTCGGCCACGGAACCGGCAGTTTACCGGTGGT
GCAAATAAATTTCAGGGTCAGTTTACCgttGGTCGCATCACCTTCGCCT
TCGCCACGAACGCTAAATTTATGGCCATTCACATCACCATCCAGTTCCA
CCAGAATCGGCACAACGCCGGTAAACAGTTCTTCACCTTTgctAACCAT
GGTTAATTCCTCCTGTTAGCCCAAAAAACGGGTATGGAGAAAGGTCTGA
TCACATTATACGAGCCGATGATTAATTGTCAAgcgcaacgcaattaatg
cgtgcaatagttatggtgaaaataaatcaaaattttattcctatcgctt
ttccgtttgtgatagttcgggcctatcggatgcagggggtattgacaga
cggaaataaaaatgttaggggcgcgggtaggtggggggggcacgtatcc
cc

The inventors provide evidence herein that that orthogonal episome may be maintained at a wide range of copy numbers. For instance, the inventors modulate the copy number from 2.5 copies per cell to 1,166 copies per cell. Thus, the cell of the first aspect, which may be a bacterial cell such as an E. coli cell, may comprise at least 2.5 copies, at least 10 copies, at least 50 copies, at least 100 copies, at least 250 copies, at least 500 copies, at least 1000 copies, or at least 1100 copies of the orthogonal episome per cell.

In a particular embodiment, the cell of first aspect is a bacterial cell that is optionally an E. coli cell, that comprises:
a gene encoding a PRD1 bacteriophage TP and a gene encoding an error-prone PRD1-compatible ODNAP; and
a linear plasmid comprising: a 5′ ITR, a sequence-of-interest, and a 3′ ITR, wherein the 5′ ITR and the 3′ ITR are compatible with the error-prone PRD1-compatible ODNAP.

The orthogonal episome may be delivered to the cell of the first aspect in a circular nucleic acid molecule, such as a circular episome. Thus, the cell of the first aspect may comprise a circular nucleic acid molecule comprising sequences that are capable of being replicated by the orthogonal DNA replication machinery. The sequences that are capable of being replicated by the orthogonal DNA replication machinery may be excised from the circular nucleic acid molecule, for instance the excision may take place within the cell. This may be referred to as in vivo excision or in vivo cutting. And so, the cell of the first aspect may comprise a circular nucleic acid molecule, wherein the circular nucleic acid molecule comprises the sequence of a linear plasmid as disclosed herein.

The circular nucleic acid molecule may be a circular episome, and the episome may be any suitable for use with the cell of the first aspect. For instance, the circular episome may be an episome that is compatible with E. coli.

The circular episome may be a bacterial artificial chromosome (BAC). The circular episome may be a circular plasmid. In an embodiment, the cell of the first aspect comprises a circular plasmid or a BAC comprising sequence that, once excised, is any of the linear plasmids disclosed herein.

The circular nucleic acid molecule may comprise at least one site that can be cleaved to excise the orthogonal sequence. For instance, the linear plasmid may be circularised at a single point that can be cleaved to linearise the orthogonal episome. The circular nucleic acid molecule may comprise two sites that can be cleaved to excise the orthogonal sequence. For instance, the circular nucleic acid molecule, such as a circular episome, may contain the sequence of a linear plasmid that is flanked by sites that can be cleaved to excise the linear plasmid.

In embodiments with one site for cleavage, the site may be positioned between a 5′ ITR and a 3′ ITR as defined herein. Thus, cleavage of the site can result in a linear plasmid comprising, in order, a 5′ ITR, a sequence-of-interest, and a 3′ ITR.

In embodiments with two sites for cleavage, the sites may be positioned adjacent to a 5′ ITR and a 3′ ITR as 50 defined herein. Thus, cleavage of the site can result in a linear plasmid comprising, in order, a 5′ ITR, a sequence-of-interest, and a 3′ ITR.

The cleavage may be inducible. Thus, the cell may comprise the circular nucleic acid molecule and cleavage may be induced under specific conditions.

In some embodiments, the sites that can be cleaved are target sites for an endonuclease or endonucleases. For instance, the sites may be targeted by RNA-guided endonucleases, such as CRISPR-Cas. However, the sites for cleavage need not be particularly limited, as long as they can be specifically cut.

The cell may comprise sequence encoding an endonuclease or endonucleases suitable for excising the orthogonal sequences from the circular nucleic acid molecule. The cell may comprise sequence encoding molecules suitable for guiding an endonuclease to a cleavage site, such as guide RNA. The endonuclease and/or guide molecules may be inducibly expressed, e.g. to allow for excision of the orthogonal sequences under particular conditions or at a desired time. The endonuclease and/or guide molecules may be encoded by the genome of the cell or by other episomes within the cell, such as a plasmid. Excision may be induced by the transfer of an episome encoding one or more components of the cleavage machinery to the cell.

Embodiments comprising circular nucleic acid molecules can be advantageous due to the easier manipulation of circular nucleic acid molecules compared to linear plasmids. For instance, a circular episome can be used to deliver a large linear plasmid, which may be hundreds of kilobases in size.

As discussed herein, the inventors provide herein an orthogonal DNA replication system suitable for use in cells such as bacterial cells (e.g. E. coli). In a second aspect, there is provided a linear plasmid comprising a 5′ ITR and a 3′ ITR, each of which is compatible with DNA replication machinery from or derived from a lytic phage of the Tectiviridea family or a lytic φ29-like virus of the Podoviridae family.

The linear plasmid may be as disclosed in connection with the first aspect. For instance, see illustrative sequences SEQ ID NOs: 7, 8, 9, 10, and 11.

The linear plasmid may be an artificial plasmid and so not naturally occurring. The linear plasmid may be isolated from the natural conditions. The linear plasmid may be an unnatural or engineered linear plasmid. The linear plasmid may comprise a sequence-of-interest. For instance, the sequence-of-interest may be a sequence to which it is desirable to apply continuous directed evolution techniques. The sequence-of-interest may be as discussed in relation to the first aspect. The sequence-of-interest may be a sequence that is not endogenous to any Podoviridae bacteriophage. The sequence-of-interest may be a sequence that is not endogenous to any Tectiviridae bacteriophage. The sequence-of-interest may be a sequence that is not endogenous to a PRD1 bacteriophage

The linear plasmid may comprise a marker. For instance, a selectable marker. The marker may be a positive marker such that cells comprising the linear plasmid may be identified or selected, or so that selection pressure can be applied to a population of cells to increase the retention of the linear plasmid.

The Podoviridae or Tectiviridae DNA replication machinery may be DNA replication machinery from any one of the lytic phages discussed herein. The DNA replication machinery may be from or derived from any of phage PRD1, phage PR3, phage PR4, phage PR5, phage L17, phage PR722, and phage BCE1. The DNA replication machinery may be as discussed in relation to the first aspect of the invention. The linear plasmid may not encode one, two, three, or all four of a TP, a DNA polymerase, an SSB, and a DSB from said DNA replication machinery.

In particular embodiments, the linear plasmid does not comprise sequences of the bacteriophage genome other than the ITRs.

In a preferred embodiment, the 5′ ITR and 3′ ITR are compatible with PRD1 bacteriophage DNA replication 50 machinery. In particular, the ITRs may be compatible with replication machinery comprising PRD1 proteins: TP, ODNAP, SSB, and optionally DSB, or variants thereof. The linear plasmid may not encode one, two, three, or all four of a TP, a DNA polymerase, an SSB, and a DSB. In particular embodiments, the linear plasmid does not comprise sequences of the bacteriophage genome other than the ITRs.

In a particular example, the linear plasmid comprises a 5′ ITR that is at least 80%, 85%, 90%, 95%, 97%, 98%, 99%, or 100% identical to SEQ ID NO: 7 or 10 and/or a 3′ ITR that is at least 80%, 85%, 90%, 95%, 97%, 98%, 99%, or 100% identical to SEQ ID NO: 8 or 11. The linear plasmid may comprise a truncation of SEQ ID NO: 7 and/or SEQ ID NO: 8 that is capable of being replicated by the orthogonal DNA replication machinery. In a particular example, the linear plasmid comprises a 5′ ITR that is at least 80%, 85%, 90%, 95%, 97%, 98%, 99%, or 100% identical to a truncation of SEQ ID NO: 7 and/or a 3′ ITR that is at least 80%, 85%, 90%, 95%, 97%, 98%, 99%, or 100% identical to a truncation of SEQ ID NO: 8, and wherein the linear plasmid is capable of being replicated by the orthogonal DNA replication machinery. The ITRs discussed in relation to cells of the first aspect may be applied to the linear plasmid of the second aspect.

In a particular embodiment, there is provided a linear plasmid comprising: a 5′ ITR, a sequence-of-interest, and a 3′ ITR, wherein the 5′ ITR and the 3′ ITR are compatible with PRD1 bacteriophage DNA replication machinery.

The cell of the first aspect and linear plasmid of the second aspect may be used in methods of maintaining a linear plasmid in a cell. Thus, in a third aspect, there is provided a method of maintaining a linear plasmid, wherein the method comprises:
i) providing a cell according to the first aspect that comprises an episome capable of being replicated by the orthogonal replication machinery, or transferring a linear plasmid according to the second aspect to a cell according to the first aspect; and
ii) incubating the cell under conditions conducive to growth.

In particular, the cell may any cell as discussed in relation to the first aspect and may comprise any features as discussed in relation to the first aspect. For instance, the cell may be a bacterial cell such as an E. coli cell.

The linear plasmid may lack 5′ phosphorylation. For instance, the linear plasmid may have been generated using non-phosphorylated primers. Thus, the method may comprise the generation of the linear plasmid using non-phosphorylated primers.

The linear plasmid may be transferred to a bacterial cell by electroporation. Alternatively, the linear plasmid may be transferred to a bacterial cell as a part of a circular nucleic acid and the linear plasmid may be excised by in vivo cutting. For instance, the cell may express an endonuclease, such as CRISPR-Cas, capable of specifically excising the linear plasmid from the circular nucleic acid. The circular nucleic acid molecule and the nature of the excision may be any as defined herein, for instance as discussed for the first aspect. Particular examples include episomes such as plasmids or BACs that are suitable for use with E. coli.

In an embodiment, the cell comprises, or is the recipient of, a circular nucleic acid molecule comprising the sequence of the linear plasmid. The linear plasmid can be excised from the circular nucleic acid molecule within the cell. For instance, an endonuclease or endonucleases within the cell may specifically cut the circular nucleic acid molecule to excise the linear plasmid. This excision may be inducible, e.g. the endonuclease or associated components—such as guide RNAs—may be inducibly expressed. Excision may be induced by the transfer of an episome encoding one or more components of the cleavage machinery to the cell.

Thus, in an embodiment, the method of the third aspect may comprise obtaining a cell comprising a circular nucleic acid molecule that comprises the sequence of the linear plasmid, and inducing excision of said linear plasmid. This step may comprise the induction of expression of cleavage machinery capable of excising the linear plasmid or the transfer of one or more components of said cleavage machinery. In such embodiments, the step of transferring a linear plasmid according to the second aspect to a cell according to the first aspect may be referred to as generating a linear plasmid according to the second aspect within a cell according to the first aspect or excising a linear plasmid according to the second aspect from a circular nucleic acid molecule within a cell according to the first aspect.

The linear plasmid may be transferred to the cell as part of a complex. The transfer may be by electroporation.

The linear plasmid for transfer may have been extracted from a cell by using techniques that do not disrupt any bound proteins. For instance, the linear plasmid for transfer may have been extracted from a cell using a technique that does not remove the bound TPs. To avoid the removal of the TPs, proteinase treatments may be avoided. Standard miniprep protocols for the purification of plasmids are suitable (e.g. QIAprep Spin Miniprep Kit, QIAGEN). Thus, the transferring of a linear plasmid according to the second aspect to a cell according to the first aspect may be the transfer of a complex comprising a TP and a linear plasmid according to the second aspect.

This is particularly relevant to embodiments that involve a cell comprising a TP and an ODNAP but not comprising an SSB or DSB.

In embodiments that involve a bacterial cell comprising genes encoding a PRD1 TP and a PRD1-compatible ODNAP, a complex comprising PRD1 TPs bound to a linear plasmid may be transferred to the bacterial cell.

In an embodiment, the method of the third aspect may comprise:
transferring a complex to a cell according to the first aspect, wherein the complex comprises TPs bound to a linear plasmid according to the second aspect; and
incubating the cell under conditions conducive to growth.

The incubation of the cell under conditions conducive to growth is exposure of the cell to conditions that maintain viability of the cell and allow for DNA replication. Such conditions are known in the art. The conditions may be selective conditions, e.g. for bacterial cells an antibiotic may be present. The conditions may select for cells that comprise the linear plasmid. For instance, the linear plasmid may comprise a marker gene or a resistance gene and the incubation of the cell may include conditions relevant to the marker gene or resistance gene.

Alternatively, the linear plasmid may comprise a sequence-of-interest for which is it anticipated that mutations to said sequence could increase the fitness of the cell during incubation. The incubation conditions may be such to enable only sub-optimal growth of the cell when comprising an unmutated sequence-of-interest. Further examples of growth and selection are provided in relation to the fourth aspect and are also applicable to the third aspect.

In a particular embodiment, there is provided a method of maintaining a linear plasmid, wherein the method comprises: i) transferring a linear plasmid to a bacterial cell, and ii) incubating the bacterial cell under conditions conducive to growth, wherein the linear plasmid comprises a 5′ ITR and a 3′ ITR, each of which is compatible with Podoviridae DNA replication machinery associated with a φ29-like lytic phage or Tectiviridae DNA replication machinery associated with a lytic phage, and wherein the bacterial cell comprises orthogonal DNA replication machinery from or derived from a Podoviridae φ29-like lytic phage or Tectiviridae lytic phage. In particular, the bacterial cell may express a Tectiviridae TP, a Tectiviridae ODNAP, a Tectiviridae SSB, and optionally a Tectiviridae DSB, or variants thereof. The orthogonal DNA replication machinery may be from or derived from any of phage PRD1, phage PR3, phage PR4, phage PR5, phage L17, phage PR722, and phage BCE1.

In another embodiment, there is provided a method of maintaining a linear plasmid, wherein the method comprises: i) transferring a linear plasmid to a bacterial cell, and ii) incubating the bacterial cell under conditions conducive to growth, wherein the linear plasmid comprises a 5′ ITR and a 3′ ITR, each of which is compatible with PRD1 bacteriophage DNA replication machinery, and wherein the bacterial cell comprises orthogonal DNA replication machinery from or derived from PRD1 bacteriophage. In particular the bacterial cell may express a PRD1 bacteriophage TP, a PRD1 bacteriophage ODNAP, a PRD1 bacteriophage SSB, and optionally a PRD1 bacteriophage DSB, or variants thereof.

In an example, the linear plasmid comprises a 5′ ITR that is at least 80%, 85%, 90%, 95%, 97%, 98%, 99%, or 100% identical to SEQ ID NO: 7 or 10 and/or a 3′ ITR that is at least 80%, 85%, 90%, 95%, 97%, 98%, 99%, or 100% identical to SEQ ID NO: 8 or 11. The linear plasmid may comprise a truncation of SEQ ID NO: 7 and/or SEQ ID NO: 8 that is capable of being replicated by the orthogonal DNA replication machinery. In a particular example, the linear plasmid comprises a 5′ ITR that is at least 80%, 85%, 90%, 95%, 97%, 98%, 99%, or 100% identical to a truncation of SEQ ID NO: 7 and/or a 3′ ITR that is at least 80%, 85%, 90%, 95%, 97%, 98%, 99%, or 100% identical to a truncation of SEQ ID NO: 8, and wherein the linear plasmid is capable of being replicated by the orthogonal DNA replication machinery.

The inventors provide herein a method of evolving a sequence-of-interest that makes use of the orthogonal DNA replication systems disclosed herein. Thus, in a fourth aspect, there is provided use of a cell according to the first aspect, a linear plasmid according to the second aspect, or a circular nucleic acid molecule of the sixth aspect for evolving a sequence-of-interest.

In an embodiment, there is provided a method of evolving a sequence-of-interest, wherein the method comprises:
i) providing a cell according to the first aspect that comprises an episome capable of being replicated by the orthogonal replication machinery, or transferring a linear plasmid according to the second aspect to a cell according to the first aspect, wherein the episome or linear plasmid comprises a sequence-of-interest and wherein the orthogonal DNA replication machinery replicates DNA with a lower fidelity than the cell's endogenous DNA replication machinery;
ii) incubating the cell under conditions conducive to growth; and
iii) exposing the cell to a selection condition.

The cell may be any cell as discussed in relation to the first aspect and may comprise any features as discussed in relation to the first aspect. For instance, the cell may be a bacterial cell such as an E. coli cell. The linear plasmid may comprise any features as discussed in relation to the first or second aspects. The linear plasmid may be transferred to the cell as discussed for the third aspect. For instance, the linear plasmid may be transferred by electroporation or may be excised from a circular nucleic acid molecule. A complex comprising the linear plasmid and TPs may be transferred to the cell.

As discussed in relation to the first aspect, replication of the linear plasmid may have a mutation rate of at least 10⁻⁹, 10⁻⁸, 10⁻⁷, 10⁻⁶, or 10^{31 5} s.p.b.

The incubation of the cell under conditions conducive to growth is exposure of the cell to conditions that maintain viability of the cell and allow for DNA replication. Such conditions are known in the art and are discussed in relation to the third aspect.

Exposing the cell to a selection condition may be exposure of the cell to conditions that prevent or reduce the growth of cells in the absence of certain characteristics. The selection condition may be present during the incubation of the cell under conditions conducive to growth, and hence these steps may be concurrent. In particular, the selection conditions may be such to enable only sub-optimal growth of the cell when comprising an unmutated sequence-of-interest. The selection conditions may be such that the fitness of the cell is increased as the sequence-of-interest is mutated in a manner that leads to the acquisition of a particular function or that leads to decrease or increase in a particular function. As a purely illustrative example, the sequence-of-interest may be a potential antibiotic resistance gene, and hence the selection condition may be an amount of antibiotic that allows 50 sub-optimal growth of bacterial cells comprising an unmutated antibiotic resistance gene but would allow improved growth of bacterial cells comprising a more effective antibiotic resistance gene. The selection condition may be applied after incubation of the cells under conditions conducive to growth for a period of time. The cells may be incubated and selected iteratively, for instance the cells may be incubated, selected, incubated, and then selected.

Another purely illustrative example of a selection condition and a sequence-of-interest is where the sequence-of-interest encodes an acyl-tRNA synthetase. The selection condition may be that the cell comprises a gene that confers reduced fitness or slower growth upon the cell in the presence of an unmutated acyl-tRNA synthetase.

The gene may allow improved fitness or growth of the cell if the acyl-tRNA synthetase is mutated to acquire a new characteristic or to improve a characteristic, such as the ability to charge a particular tRNA with a particular substrate.

Alternatively, the selection condition may comprise sorting based on the expression level of a marker gene. For instance, the sequence-of-interest may encode a promoter operably linked to GFP. Cells with an above threshold level of GFP, or with a high level of GFP within the culture, may be sorted and retained. Potentially such cells may be re-entered into the method of evolving a sequence-of-interest. Such techniques are also compatible with other sequences-of-interest, for instance GFP gene may be arranged to be decoded only if an enzyme, such as an acyl-tRNA, is mutated to acquire a new characteristic or to improve a characteristic. The sequence-of-interest may, itself, act as a marker gene and may acquire or increase marker function during the evolving process.

The above examples are purely illustrative, and the invention is compatible with a wide range of sequences-of-interest, selection conditions, and desired functionality. These functionalities include enzyme activity, ribozyme activity, binding properties of proteins or aptamers, promoter or enhancer activity, capability of a nucleic acid or polypeptide to be bound by a known binding-molecule, etc.

The method of the fourth aspect may further comprise identifying the sequence of a sequence-of-interest from a cell meeting the selection condition. For instance, a cell that has an above-threshold level of fitness in the presence of the selection condition, a cell for which the selection condition is an above-threshold expression of a marker, or a cell for which the selection condition is an above-threshold detectable property or functionality. Such cells may be isolated and sequence information for the sequence-of-interest may be obtained.

The method may then comprise the making of a nucleic acid or polypeptide according to said identified sequence.

In an embodiment, there is provided a method of evolving a sequence-of-interest, wherein the method comprises:
i) providing a bacterial cell comprising a linear plasmid,
   wherein the linear plasmid comprises a 5′ ITR, a sequence-of-interest, and a 3′ ITR, wherein the 5′ and 3′ ITR are compatible with DNA replication machinery from or derived from a lytic phage of the Tectiviridea family or a lytic φ29-like virus of the Podoviridae family, and
   wherein the bacterial cell comprises orthogonal DNA replication machinery from or derived from the phage;
ii) incubating the bacterial cell under conditions conducive to growth; and
iii) exposing the bacterial cell to a selection condition.

The bacterial cell may express a Podoviridae or Tectiviridae TP and a Podoviridae or Tectiviridae ODNAP, or variants thereof. In particular, the bacterial cell may express a Podoviridae or Tectiviridae TP, a Podoviridae or Tectiviridae ODNAP, a Podoviridae or Tectiviridae SSB, and optionally a Podoviridae or Tectiviridae DSB, or variants thereof. The bacterial cell may express a Tectiviridae TP and a Tectiviridae ODNAP, or variants thereof.

The bacterial cell may express a Tectiviridae TP, a Tectiviridae ODNAP, and a Tectiviridae SSB, or variants thereof. In particular, the bacterial cell may express a Tectiviridae TP, a Tectiviridae ODNAP, a Tectiviridae SSB, and a Tectiviridae DSB, or variants thereof. In examples, the Tectiviridae family member may be any one 50 of phage PRD1, phage PR3, phage PR4, phage PR5, phage L17, phage PR722, and phage BCE1.

In an embodiment, there is provided a method of evolving a sequence-of-interest, wherein the method comprises:
i) providing a bacterial cell comprising a linear plasmid,
   wherein the linear plasmid comprises a 5′ ITR, a sequence-of-interest, and a 3′ ITR, wherein the 5′ and 3′ ITR are compatible with PRD1 bacteriophage DNA replication machinery, and
   wherein the bacterial cell comprises orthogonal DNA replication machinery from or derived from PRD1 bacteriophage;
ii) incubating the bacterial cell under conditions conducive to growth; and
iii) exposing the bacterial cell to a selection condition.

The bacterial cell may express a PRD1 bacteriophage TP and a PRD1 bacteriophage ODNAP, or variants thereof. The bacterial cell may express a PRD1 bacteriophage TP, a PRD1 bacteriophage ODNAP, and a PRD1 bacteriophage SSB, or variants thereof. the bacterial cell may express a PRD1 bacteriophage TP, a PRD1 bacteriophage ODNAP, a PRD1 bacteriophage SSB, and a PRD1 bacteriophage DSB, or variants thereof. In particular, the bacterial cell may express a PRD1 bacteriophage TP, a PRD1 bacteriophage ODNAP, a PRD1 bacteriophage SSB, and optionally a PRD1 bacteriophage DSB, or variants thereof.

In a fifth aspect, there is provided a method of making a polypeptide or nucleic acid, wherein the method comprises: i) providing a sequence-of-interest identified by a method of the fourth aspect, and ii) producing a polypeptide or nucleic acid according to said sequence.

In a sixth aspect, there is provided a circular nucleic acid molecule comprising the sequence of a linear plasmid of the second aspect.

The circular nucleic acid molecule may be a circular episome. The circular episome may be any episome suitable for use with the cell of the first aspect. For instance, the circular episome may be an episome that is compatible with E. coli. The circular episome may be a bacterial artificial chromosome (BAC). The circular episome may be a circular plasmid.

The sequence of the linear plasmid may be excised from the circular nucleic acid molecule and the excision may take place within a cell. Thus, the circular nucleic acid molecule may be configured so that the linear plasmid can be excised in vivo.

The circular nucleic acid molecule may comprise at least one site that can be cleaved to excise the linear plasmid.

For instance, the linear plasmid may be circularised at a single point that can be cleaved to linearise the linear plasmid. The circular nucleic acid molecule may comprise two sites that can be cleaved to excise the orthogonal sequence. For instance, the circular nucleic acid molecule may contain the sequence of a linear plasmid that is flanked by sites that can be cleaved to excise the linear plasmid. In embodiments with one site for cleavage, the site may be positioned between a 5′ ITR and a 3′ ITR as defined herein. In embodiments with two sites for cleavage, the sites may be positioned adjacent to a 5′ ITR and a 3′ ITR as defined herein. Thus, cleavage of the site can result in a linear plasmid comprising, in order, a 5′ ITR, a sequence-of-interest, and a 3′ ITR.

In a particular embodiment, there is provided a circular episome suitable for use with E. coli, wherein the circular episome comprises the sequence of a linear plasmid of the second aspect, and wherein the circular episome is configured such that the linear plasmid may be excised by an endonuclease or endonucleases.

In some embodiments, the sites that can be cleaved are target sites for an endonuclease or endonucleases. For instance, the sites may be targeted by RNA-guided endonucleases, such as CRISPR-Cas. However, the sites for cleavage need not be particularly limited, as long as they can be specifically cut.

In a seventh aspect, there is provided a DNA polymerase comprising any of the mutations disclosed herein.

The DNA polymerase may be at least 80%, 85%, 90%, 95%, 97%, 98%, 99%, or 100% similar or identical to SEQ ID NO: 2 and comprise any of the mutations disclosed herein.

The DNA polymerase may be at least 80%, 85%, 90%, 95%, 97%, 98%, 99%, or 100% similar or identical to SEQ ID NO: 2 and comprise any one, or any combination, of D17A, T201, H70R, N71D, D76A, Y127A, Q275L, 1291V, D325V, K329R, L341R, Y347C, N353K, E364G, 1367T, Q420H, L437S, and Q446L.

The DNA polymerase may be at least 80%, 85%, 90%, 95%, 97%, 98%, 99%, or 100% similar or identical to SEQ ID NO: 2, and comprise any one, or any combination, of D17A, T201, H70R, N71D, D76A, Y127A, and L341R.

The DNA polymerase may be at least 80%, 85%, 90%, 95%, 97%, 98%, 99%, or 100% similar or identical to SEQ ID NO: 2, and comprise any one, or any combination, of Y347C, 1367T, 1291V, D325V, N353K, E364G, Q420H, Q275L, K329R, L437S, and Q446L.

The DNA polymerase may be at least 80%, 85%, 90%, 95%, 97%, 98%, 99%, or 100% similar or identical to SEQ ID NO: 2 and comprise any one, or any combination, of Y347C, D325V, K329R, and N353K.

The DNA polymerase may be at least 80%, 85%, 90%, 95%, 97%, 98%, 99%, or 100% similar or identical to SEQ ID NO: 2 and comprise any one the following mutations or sets of mutations: T20I, N71D, N71D & L341R, H70R, D17A, D76A, D17A & D76A, D17A & D76A & L341R, and Y127A.

The DNA polymerase may be at least 80%, 85%, 90%, 95%, 97%, 98%, 99%, or 100% similar or identical to SEQ ID NO: 2 and comprise any one the following mutations or sets of mutations: i) Y127A, Y347C, and 1367T; ii) Y127A, 1291V, and D325V; iii) Y127A, Y347C, N353K, E364G, and Q420H; iv) Y127A, Q275L, K329R, Y347C, and L437S; or v) Y127A, Y347C, and Q446L.

The DNA polymerase may be capable of replicating a linear plasmid. The DNA polymerase may be capable of replicating a linear plasmid of the second aspect. The DNA polymerase may be error prone. The DNA polymerase may have an error rate as discussed for the first aspect or for the Examples.

In an eight aspect, there is provided a nucleic acid sequence encoding a DNA polymerase of the seventh aspect.

In a ninth aspect, there is provided use of a DNA polymerase of the seventh aspect.

In an embodiment, the DNA polymerase is used in a method of maintaining a linear plasmid, as discussed in relation to the third aspect. In an embodiment, the DNA polymerase is used in a method of evolving a sequence-of-interest, as discussed in relation to the fourth aspect.

Sequence comparisons can be conducted with the aid of readily available sequence comparison programs. These publicly and commercially available computer programs can calculate sequence identity between two or more sequences.

The skilled technician will appreciate how to calculate the percentage identity between two nucleic sequences or two amino acid sequences. In order to calculate the percentage identity, an alignment of the two sequences must first be prepared, followed by calculation of the sequence identity value. The percentage identity for two sequences may take different values depending on: (i) the method used to align the sequences, for example, the Needleman-Wunsch algorithm (e.g. as applied by Needle (EMBOSS) or Stretcher (EMBOSS), the Smith-Waterman algorithm (e.g. as applied by Water (EMBOSS)), or the LALIGN application (e.g. as applied by Matcher (EMBOSS); and (ii) the parameters used by the alignment method, for example, local versus global alignment, the matrix used, and the parameters applied to gaps. In a particular embodiment, the sequence identities disclosed herein may be calculated based on a global alignment of the relevant feature.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length-dependent. Therefore, the shorter a pair of sequences is, the higher the sequence identity one may expect to occur by chance.

A calculation of percentage identities between two nucleic acid sequences may then be calculated from such an alignment as (N/T)*100, where N is the number of positions at which the sequences share an identical residue, and T is the total number of positions compared including gaps but excluding overhangs.

The sequence alignment may be a pairwise sequence alignment. Suitable services include Needle (EMBOSS), Stretcher (EMBOSS), Water (EMBOSS), Matcher (EMBOSS), LALIGN, or GeneWise. In an example, the identity between two amino acid sequences may be calculated using the service Needle (EMBOSS) set to the default parameters, e.g. matrix (BLOSUM62), gap open (10), gap extend (0.5), end gap penalty (false), end gap open (10), and end gap extend (0.5). In another example, the identity between two amino acid sequences may be calculated using the service Matcher (EMBOSS) set to the default parameters, e.g. matrix (BLOSUM62), gap open (14), gap extend (4), alternative matches (1). In an example, the identity between two nucleic acid sequences may be calculated using the service Needle (EMBOSS) set to the default parameters, e.g. matrix (DNAfull), gap open (10), gap extend (0.5), end gap penalty (false), end gap open (10), and end gap extend (0.5). In another example, the identity between two nucleic acid sequences may be calculated using the service Matcher (EMBOSS) set to the default parameters, e.g. matrix (DNAfull), gap open (16), gap extend (4), alternative matches (1).

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made to the Examples, which are not intended to limit the invention in any way.

### EXAMPLES

### Abstract

The evolution of new function in living organisms is slow, and fundamentally limited by their error threshold—the number of mutations a genome can tolerate before the organism is non-viable. Here we boot up a synthetic, stable orthogonal replicon in E. coli, the workhorse of molecular biology, using selected genetic elements from a lytic phage which replicates uncontrollably and lyses cells in less than 60 minutes. The orthogonal replicon is not copied by host DNA polymerases, but is selectively copied by an orthogonal DNA polymerase (O-DNAP), which does not copy the genome. The orthogonal replicon is composed of linear double stranded DNA, can carry diverse cargo of at least 16.5 kb, and simply requires 18 bp flanking sequences that function as origins of replication. Orthogonal replicons can be generated by PCR and booted up by electroporation into cells. We control the copy number of orthogonal replicons over a 465-fold range and show that they can be stably maintained for hundreds of generations, as necessary for continuous evolution in cells. We design mutant O-DNAPs that increase the mutation rate of the orthogonal replicon by two to four orders of magnitude. Importantly, these O-DNAPs stably 50 increase the mutation rate on the orthogonal replicon above the critical error threshold for the E. coli genome, but do not mutate the genome; moreover, they have remarkably even mutagenic signatures, which favours unbiased evolutionary trajectories. We demonstrate the utility of our system for accelerated continuous evolution by rapidly evolving cellular phenotypes in multiple replicates. We evolve the tetracycline resistance gene to realize a 150-fold increase in resistance to tigecycline, 74-fold higher than previously reported mutants, in 12 days. And, we evolve a 10³-fold increase in GFP fluorescence in less than five days.

### Example 1—EcORep

An orthogonal replication system in Escherichia coli (EcORep) could provide a platform that combines the advantages from both currently dominant continuous evolution platforms: i) unbiased cargo propagation and selection and ii) fast generation times.

Here we use genetic elements from a lytic phage, which undergoes uncontrolled replication and lyses cells, to generate a stable orthogonal replication system in E. coli. We identify the genes necessary for in vivo replication of the lytic phage and separate these from the genes that encode proteins responsible for cell lysis. We add the phage replication genes, in a regulatable synthetic replication operon, to the genome of E. coli and show that we can boot up an orthogonal replication system in the resulting cells; remarkably, we can achieve this by simply transforming the cells with an orthogonal replicon (composed of linear double stranded DNA bearing the terminal repeat sequence derived from the phage genome that act as orthogonal origins of replication). We improve the efficiency with which we boot up the orthogonal replication system 680-fold, and enable linear replicons of at least 16.5 kb to be established in vivo. We demonstrate that we can control the copy number of the orthogonal replicon over a 465-fold range, by controlling the expression of the synthetic replication operon. We show that the system is stable, and can be maintained for hundreds of generations, as necessary for continuous evolution in cells. We design mutant O-DNA polymerases that increase the mutation rate by two to four orders of magnitude. Importantly, these O-DNA polymerases stably increase the mutation rate on the orthogonal replicon above the critical error threshold for the E. coli, without mutating the genome; they also have remarkably even mutagenic signatures. We demonstrate the utility of our system by rapidly evolving cellular phenotypes. We evolve the tetracycline resistance gene to realize a 150-fold increase in resistance to tigecycline, 74-fold higher than a previously reported mutant, in 12 days. And, we evolve a 10³-fold increase in GFP fluorescence in less than five days.

Herein is provided the EcORep system based on the linear genome and the replication machinery of E. coli phage PRD1 from the Tectiviridae family. The EcORep system relies on the PRD1-derived orthogonal linear plasmid (LP) that requires four proteins organised in one operon for its replication: an orthogonal DNA polymerase (ODNAP), a terminal protein (TP), a single-stranded DNA-binding protein (SSB), and a double-stranded DNA-binding protein (DSB). The terminal protein is covalently linked to each 5′-end of the LP.

Since PRD1 is a lytic phage, the replication of its genome is uncontrolled. In the EcORep system, however, stable replication of the LP is required. We therefore tested different promoters for controlled expression of the replication components by integration of the operon in the E. coli genome. The final construct encoding the LP replication machinery controlled by an inducible IPTG promoter was then electroporated as PCR product into cells. We confirmed stable in vivo replication of LP based on phenotype (antibiotic resistance and GFP fluorescence), genotype (PCR verification) and extraction of the LP. The copy number will be tested using Q-PCR, and it is expected that the LP copy number will change under different concentrations of IPTG.

With the technology at hand, any target sequence can be stably replicated and expressed independently from the E. coli genome.

For a continuous directed evolution system in vivo, diversification of the target sequence in each replication cycle is required. This may be achieved by engineering ODNAP to exhibit lower replication fidelity. As proof-of-principle, we envision exemplifying the utility of this system by directed evolution of an orthogonal acyl-tRNA synthetase. Positive and negative selection can be achieved through addition of non-canonical amino acids and induction of selection pressure. Future applications are far-reaching from genome engineering to the directed evolution of medicinally relevant molecules such as nanobodies.

### Example 2—Stability of EcORep

To test the stability of the EcORep system, cells will be passaged and the stability tested through flow cytometry. The cultures of the strains were serially diluted at 1/1000 (approximately 2¹⁰) and cultured for 12 h both with or without antibiotic selection. Flow cytometry was used to determine the proportion of cells expressing GFP and then to further represent the proportion of cells with plasmids. At the same time, the commonly used circular plasmid with ColE1 replication origin were used as controls. We found the LP is much more stable than the circle plasmids in this case.

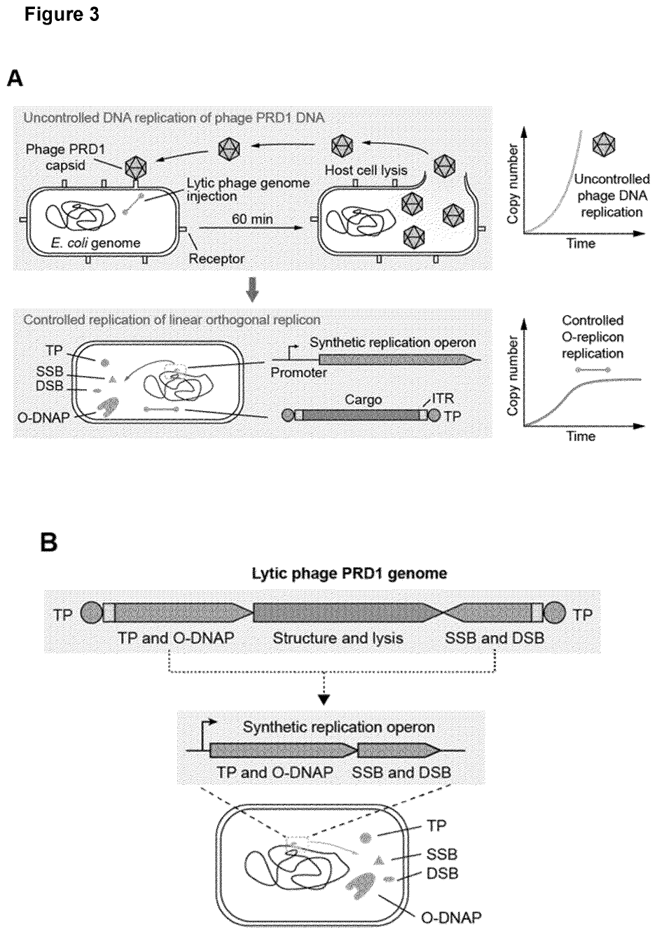

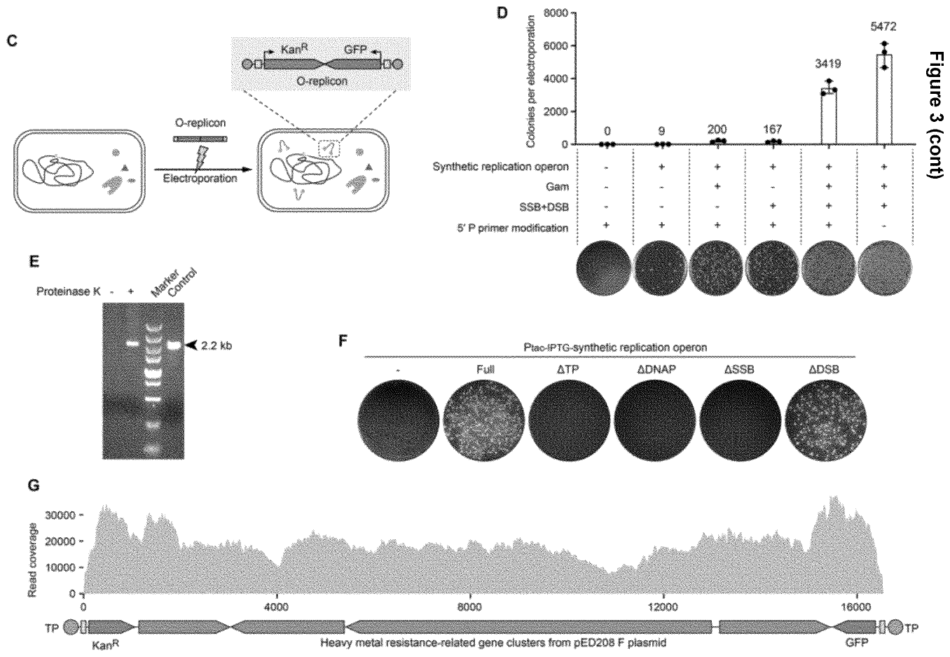

Example 3—Booting up a synthetic linear DNA replication system in vivo PRD1 is a lytic phage that infects E. coli, undergoes uncontrolled replication, and lyses cells in 60 minutes (FIG. 3A, FIG. 9) (20). Its linear double stranded genome encodes at least 25 gene products from five annotated operons under the control of eight annotated promoters and terminators (21). The ends of the linear genome are composed of inverted terminal repeats that form the binding site for the terminal protein (TP) and function as origins of replication (FIG. 3B) (22). The early operons contain the genes responsible for replication of the PRD1 genome.

The left early operon encodes the TP and the DNAP, and the right early operon encodes phage single strand and double strand DNA binding proteins (SSB and DSB, respectively). The central operons contain the genes that encode the remaining structural and lytic protein components of PRD1.

To generate a synthetic system for the controlled replication of a linear replicon (FIG. 3A), we separated the four genes that we hypothesized might be essential for in vivo replication of the PRD1 genome from the structural and lytic genes and combined them into a single synthetic replication operon, controlled by an IPTG inducible promoter (PtacJPTG) (FIG. 3B). We hypothesized that this synthetic replication operon might be sufficient to direct the replication of any linear double stranded DNA flanked by PRD1 terminal repeat sequences in E. coli, without leading to the uncontrolled replication and cell lysis mediated by the parent phage. We integrated the synthetic replication operon into the genome of E. coli to create a strain primed for replicating a linear replicon composed of linear double stranded DNA flanked by PRD1 terminal repeat sequences. We induced expression of the synthetic replication operon in these cells and made them electrocompetent.

We created a Kan^{R}-GFP linear replicon composed of a kanamycin resistance gene and a GFP gene under the control of constitutive promoters, flanked by 110 bp PRD1 inverted terminal repeat sequences on each end; the sequence was amplified by PCR using 5′ phosphorylated primers (FIG. 3C). We electroporated this replicon into E. coli bearing the synthetic replication operon in their genome and plated the cells on agar plates containing IPTG, to express the operon, and kanamycin, to maintain the replicon. We obtained a small number of colonies (9±4) per 100 μL of competent cells that grew on kanamycin and exhibited GFP fluorescence; consistent with the linear replicon being present in cells (FIG. 3D). We did not observe growth on kanamycin when the linear replicon was electroporated into cells that did not contain the synthetic replication operon; this demonstrated that the synthetic replication operon is necessary for the maintenance of the linear replicon (FIG. 3D).

To explicitly demonstrate the presence of the linear replicon in cells, we lysed the cells and performed a standard plasmid extraction protocol. Upon treating the extract with proteinase K, to remove the covalently bound TP from the ends of the linear replicon, we directly visualized a band corresponding to the linear replicon double stranded DNA on agarose gel electrophoresis (FIG. 3E). Taken together, our experiments demonstrated that we have created a linear replicon that requires the synthetic replication operon for its maintenance and replication.

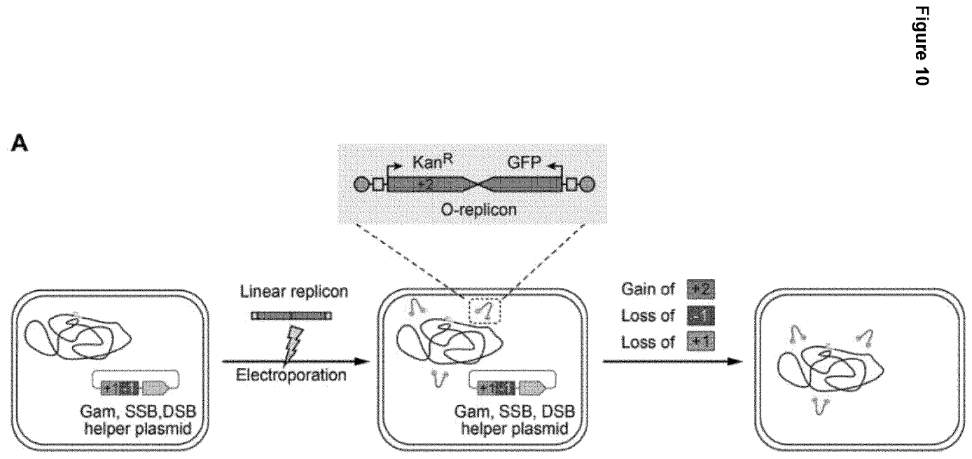

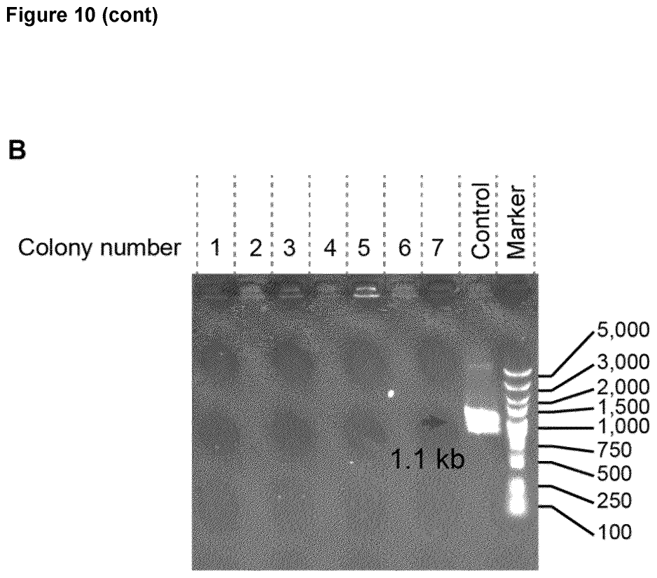

As we obtained a small number of colonies per 100 μL of competent cells, we investigated strategies to increase the efficiency with which we could boot up in vivo replication of the linear replicon. We hypothesized that expression of the Gam protein from the lambda phage, that inhibits host nucleases (RecBCD and sbcCD) and thereby protects linear double stranded DNA from degradation (23), might increase the efficiency with which we can boot up in vivo replication of the linear replicon. We found that expression of Gam in cells increased the number of colonies recovered approximately 20-fold, and we also found that overexpression of plasmid-encoded PRD1 SSB and DSB increased colony formation comparably to Gam (FIG. 3D). Overexpressing Gam, PRD1 SSB and DSB together increased the number of colonies 370-fold with respect to the original system, and using non-phosphorylated primers with overexpressed Gam, SSB and DSB, increased the number of colonies 680-fold with respect to the original system (FIG. 3D). The helper plasmids used to express Gam, SSB and DSB were easily cured from cells once the in vivo replication of the linear replicon was established (FIG. 10).

We electroporated the Kan^{R}-GFP linear replicon into E. coli cells transformed with a single-copy plasmids bearing a synthetic replication operon in which one of each of the four genes in the operon (encoding the TP, DNAP, SSB and DSB) was disrupted (FIG. 3F). These experiments demonstrated that the DNAP, TP and SSB, but not the DSB, were necessary for maintenance of the linear replicon. As the linear replicon is replicated by the PRD1-derived DNAP, but not the host DNAPs, we refer to it as an orthogonal replicon.

Using the most efficient transformation system we booted up in vivo replication for a 16.5 kb orthogonal replicon (FIG. 3G). This demonstrates that we can use the system for large cargos that are beyond the scope of viral systems.

### Example 4—Orthogonal Replicon is Stably Inherited

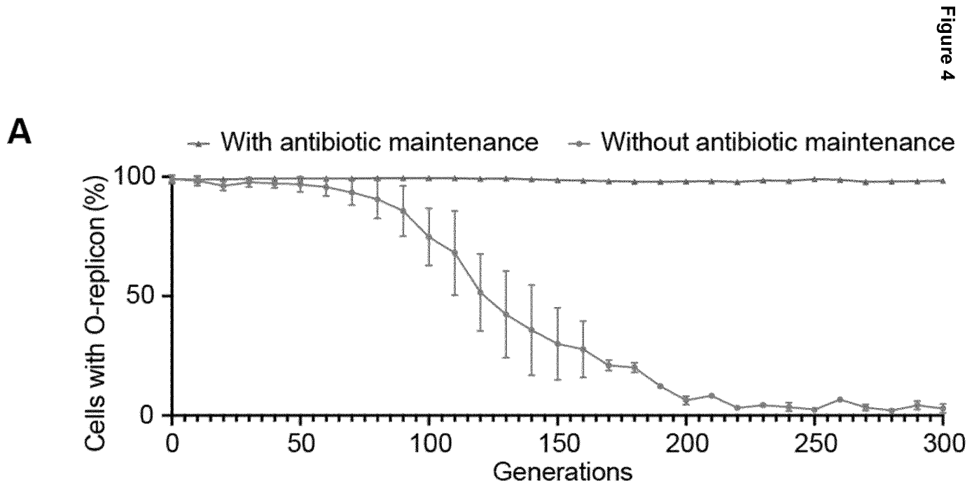

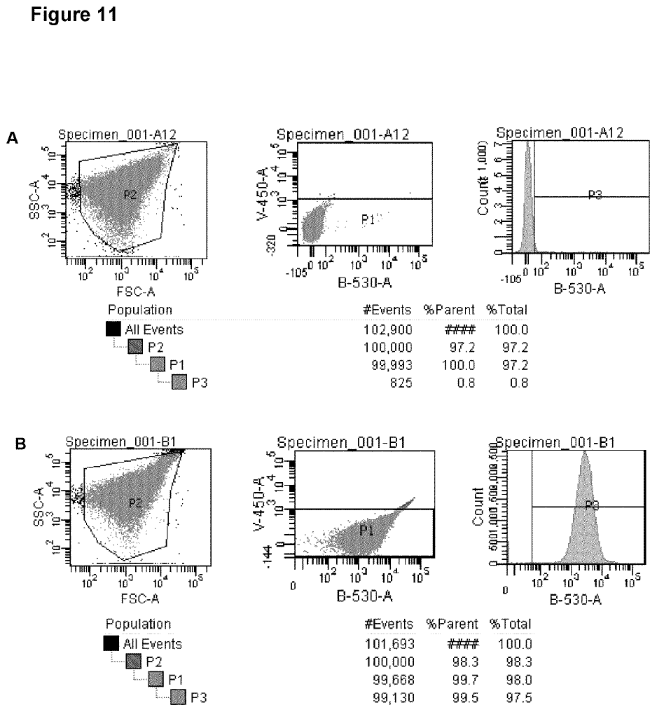

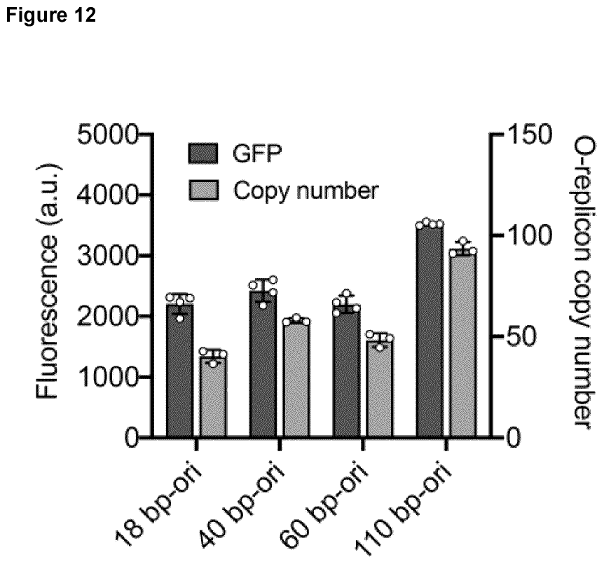

To investigate the stability of the orthogonal replicon through many cell divisions, we followed the percentage of cells that maintain the Kan^{R}-GFP orthogonal replicon, as judged by GFP fluorescence in FACS, over 100 generations (FIG. 4A). We did so for cells where the genomically integrated synthetic replication operon was under the control of different promoters (FIG. 4B, FIG. 11). In the presence of kanamycin, the Kan^{R}-GFP orthogonal replicon was stably maintained for 100 generations (FIG. 4B, FIG. 11). In the absence of kanamycin, GFP fluorescence was maintained for approximately 50 generations before it began to decay (FIG. 4B). These experiments demonstrated that the orthogonal replicon can be stably maintained in cells for many generations, as required for directed evolution using orthogonal replication systems.

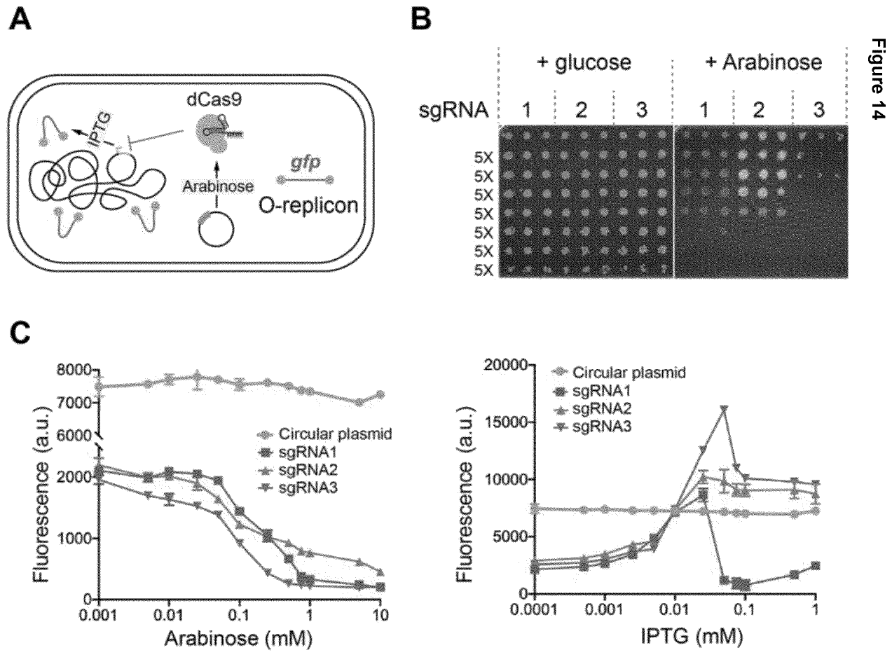

Example 5—Defining minimal origins of replication for the orthogonal replicon To establish the minimal origin length required for booting up an orthogonal replicon, we prepared linear DNA with iteratively truncated inverted terminal repeats (FIG. 3C). We found that replicons with 60, 40 or 18 bp truncated inverted terminal repeats could readily be booted up and maintained under selection for at least 100 generations (FIG. 3D, FIG. 14). Linear DNA bearing 10 bp of the inverted terminal repeats did not enable booting up of the replicon. An alignment of the left origin of the PRD1 phage to the left origins of other Tectiviridae phages that prey on E. coli revealed this minimal 18 bp sequence to be conserved (FIG. 9B).

Example 6—Maintaining multiple distinct orthogonal replicons simultaneously To test whether multiple distinct orthogonal replicons could be maintained in the same cell simultaneously, we sequentially booted up orthogonal replicons carrying different selection 30 markers (Kan^{R}, Cm^{R}, Amp^{R} and/or Tet^{R}) (FIG. 4E). We found that at least three orthogonal replicons could be maintained simultaneously, under selection, for at least 100 generations (FIG. 4F). This may enable the directed evolution of multi-gene pathways without the requirement for these genes to be on the same contiguous stretch of DNA.

### Example 7—Controlling Orthogonal Replicon Copy Number

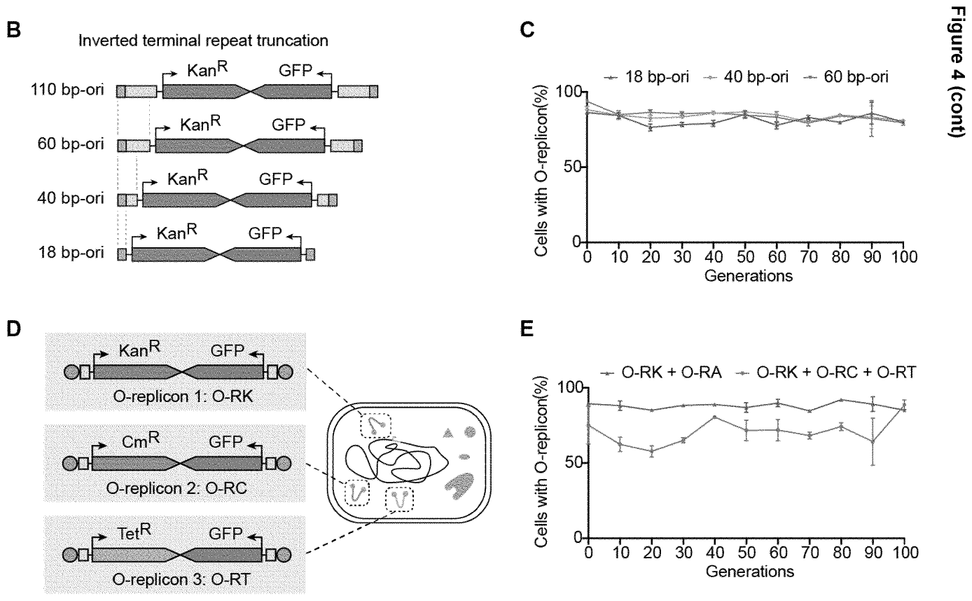

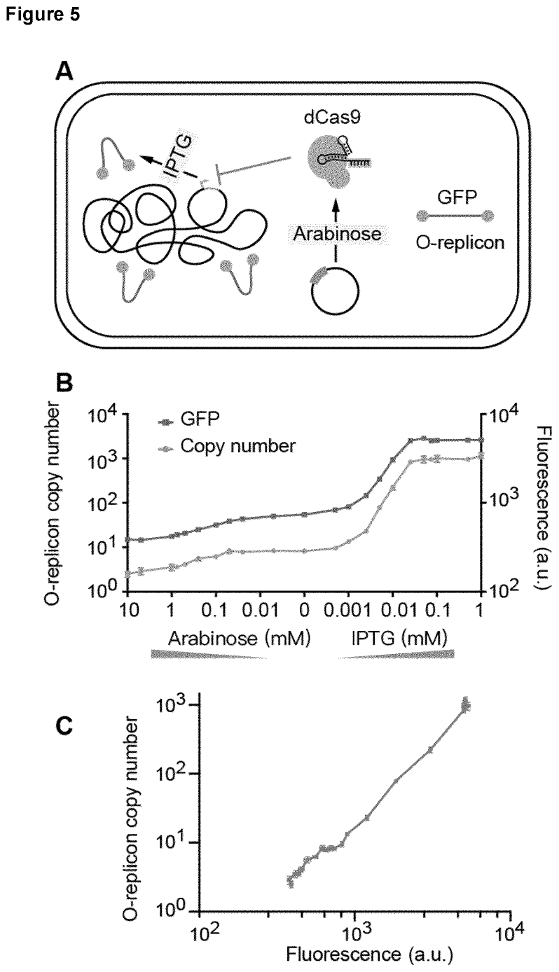

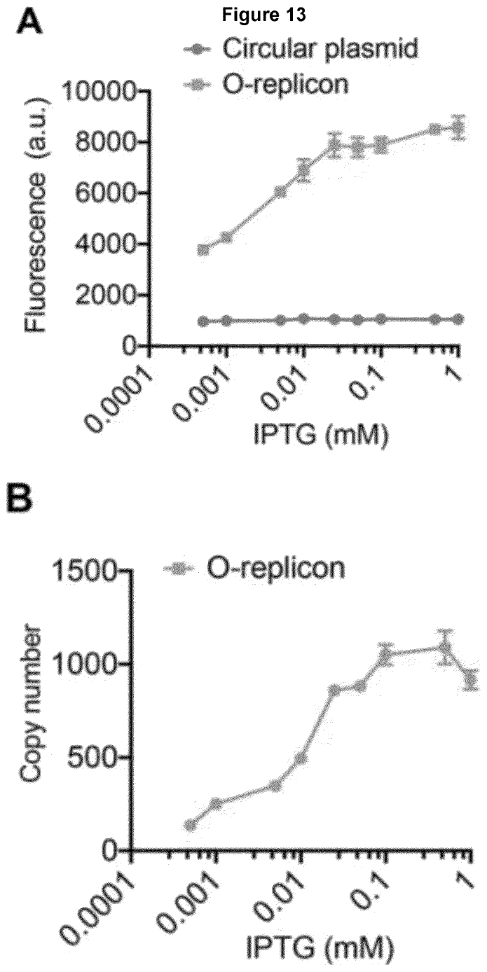

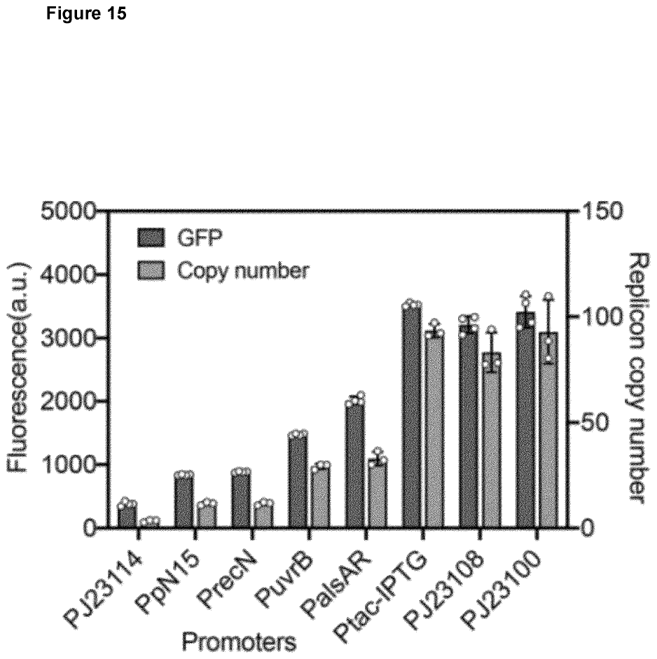

Next, we modulated the copy number of an orthogonal replicon (expressing GFP from a constitutive promoter) in cells containing the IPTG inducible synthetic replication operon (FIG. 5A, FIG. 13). Cells also contained an arabinose inducible dCas9 targeted to repress the IPTG responsive Ptac promoter on the synthetic replication operon (FIG. 14). By addition of arabinose or IPTG to cells—we modulated the copy number of the orthogonal replicon over 465-fold, from 2.5 copies per cell to 1,166 copies per cell (FIG. 5B). We observed an increase in fluorescence, resulting from GFP expression, with increasing orthogonal replicon copy number (FIG. 5C). In additional experiments where we varied the identity of the promoter used to drive expression of the synthetic replication operon; we again observed an increase in fluorescence with increasing orthogonal replicon copy number (FIG. 15). In all cases, we further validated the precise orthogonal replicon copy numbers via qPCR. These experiments demonstrated that we can regulate the copy number of the orthogonal replicon, and therefore gene expression from the orthogonal replicon, over a wide dynamic range.

Example 8—Error-prone ODNAP For a continuous directed evolution system in vivo, diversification of the target sequence in each replication cycle is required. This may be achieved by engineering ODNAP to exhibit lower replication fidelity. To obtain different error-prone orthogonal DNA polymerase (ODNAP) variants, rational design will be performed according to structure analysis and homology analysis. A mutation rate of 10⁻⁵ is expected. Also, it is expected that the genomic mutation rate will be the same in the presence of error-prone ODNAP.

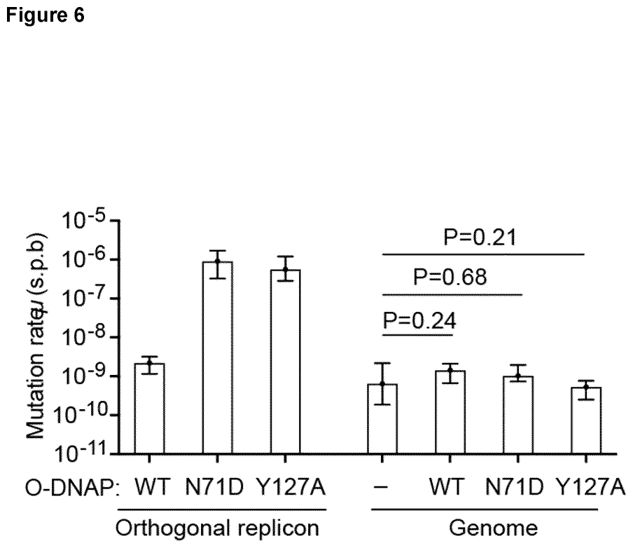

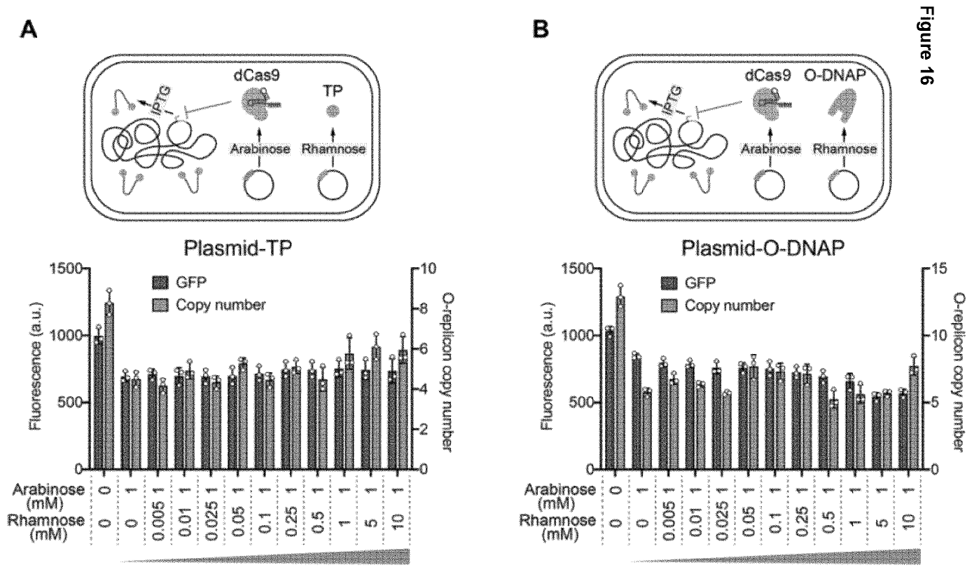

Example 9—Mutagenic DNA polymerases for orthogonal replicon To measure the mutation rate in replication of the orthogonal replicon, we created a Kan^{R}_Cm^{R}(Gln38TAG) orthogonal replicon that contains an amber stop codon (TAG) at position 38 of the chloramphenicol resistance gene (Cm^{R}). We introduced this orthogonal replicon into cells containing a synthetic replication operon with a WT DNAP. We transformed these cells with a plasmid encoding an arabinose inducible dCas9, which upon addition of arabinose represses the synthetic replication operon, and a plasmid encoding TP and a DNAP of interest under the control of a rhamnose promoter (FIG. 16). We grew cells in kanamycin, apramycin (to maintain the DNAP-encoding plasmid), arabinose and rhamnose, to switch from using the genomically encoded WT DNAP to primarily using the plasmid-encoded DNAP of interest for replicating the orthogonal replicon. After ten generations, we measured the fraction of Cm-resistant cells, resulting from point mutations that convert the TAG stop codon to sense codons; as a single copy of the intact Cm^{R} gene can confer chloramphenicol resistance we also measured the copy number of the orthogonal replicon (FIG. 17). We used this information to calculate the mutation rate per generation per base pair for the DNAP at the TAG codon in the orthogonal replicon (p1, FIG. 6A, FIG. 17). The mutation rate for the WT DNAP was 1.0×10⁻⁹ mutations per generation per base pair. We designed nine DNAPs (FIG. 17) with the goal of increasing the mutation rate of the orthogonal replicon. The mutant polymerases increased the mutation rate to between 2.6×10⁻⁸ to 8.7×10⁻⁶ mutations per generation per base pair (FIG. 6A, FIG. 17). We focused on two mutant polymerases, N71D and Y127A, with mutation rates of approximately 2.0×10⁻⁷ mutations per generation per base pair, as these mutant polymerases—unlike the more mutagenic polymerases—supported linear orthogonal replicon copy numbers comparable to the WT polymerase.

### Example 10—O-DNA Polymerases do not Copy the Genome

To measure the genomic mutation rate in cells containing each DNAP (WT, N71D, and Y127A), we introduced a Cm^{R}(Gln38TAG) gene into the genome of strains containing the synthetic replication operon, and switched from the genomically encoded WT DNAP to the plasmid encoded DNAP of interest, as described above. After ten generations, we measured the fraction of Cm-resistant cells, resulting from point mutations that convert the TAG stop codon in the genome to sense codons, and calculated the genomic mutation rate (μl) per generation per base pair at the TAG codon. The genomic mutation rates with each mutant DNAP were indistinguishable from the genomic mutation rate in unmodified WT cells (FIG. 6A). Moreover, the genomic mutation rates we measured (5.5×10⁻¹⁰ mutations per generation per base pair) were comparable to those previously reported for E. coli (24, 25). We conclude that the DNAP mutants can increase the mutation rate for replication of the orthogonal replicon without affecting the mutation rate of the genome, which is replicated by host polymerases. The mutation rate for replication of the orthogonal replicon by the N71D, and Y127A mutant polymerases is three orders of magnitude higher than the mutation rate of the genome. Overall, we conclude that the DNAP for the orthogonal replicon is an O-DNAP and the orthogonal replicon and synthetic replication operon constitute (which contains the O-DNAP) an E. coli orthogonal replication system (EcORep).

### Example 11—Mutant O-DNAPs Exceed the Error Threshold of E. coli and have Minimal Mutational Bias

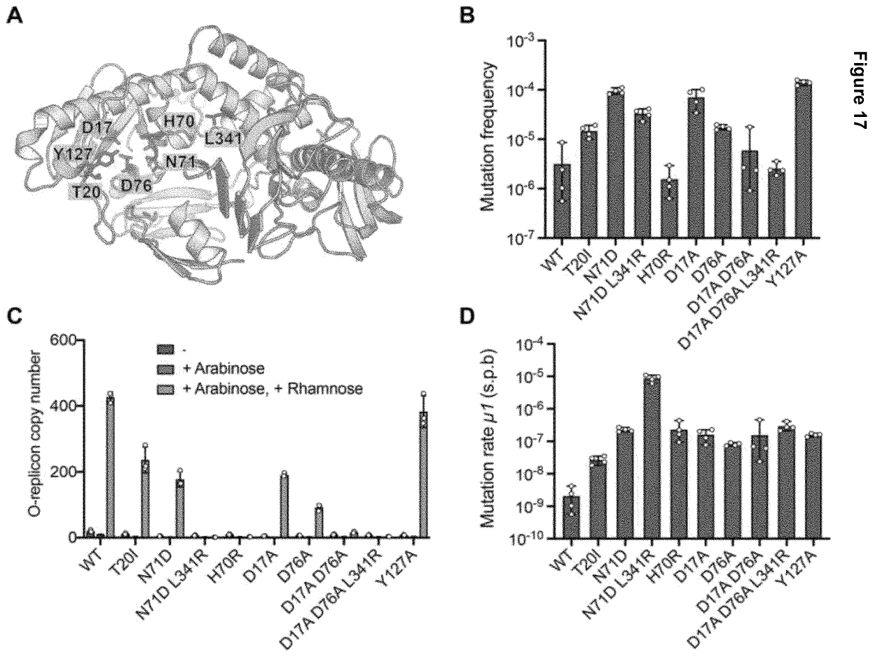

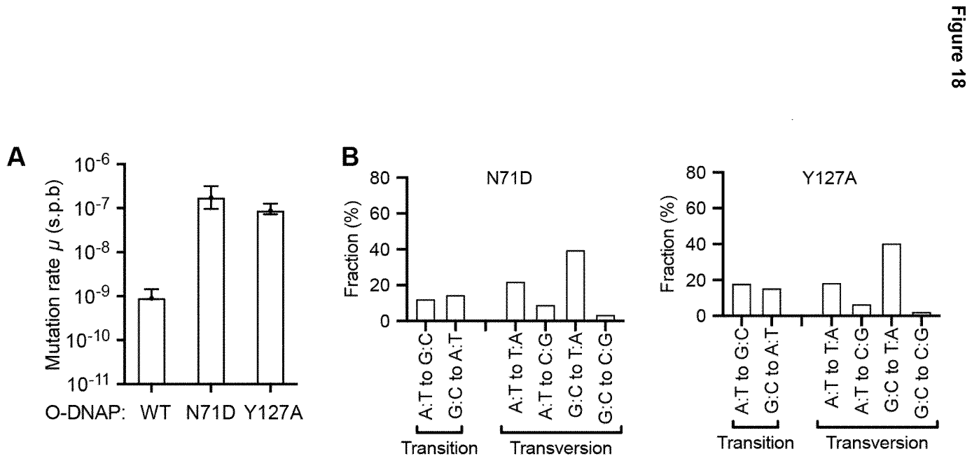

Next, we further characterized: 1) the mutation rate across the orthogonal replicon, and 2) the spectrum of mutations at each base, for the selected O-DNAPs. We transformed cells containing the synthetic replication operon with the Kan^{R}_Cm^{R}(Gln38TAG) or Kan^{R}-GFP orthogonal replicon, and switched from replicating the orthogonal replicon using the genomically encoded WT O-DNAP to primarily using the plasmid encoded O-DNAP of interest to replicate the orthogonal replicon, as described above. We plated cells after 10 generations' growth. We Sanger sequenced 96 colonies from plates (with 20 μg ml⁻¹ chloramphenicol) from the mutation rate measurement experiments for mutant N71D and Y127A, and calculated the mutational spectrum of transition (A:T to G:C) and transversion (A:T to T:A, A:T to C:G, G:C to T:A, G:C to C:G). To measure the transition (G:C to A:T) data, we designed a Cm^{R}(S145G) orthogonal replicon, a single base pair mutation from G (GGT) to S (AGT) or R (CGT) can confer resistance to 150 μg ml⁻¹ chloramphenicol. We introduced this orthogonal replicon into cells containing a synthetic replication operon with a WT DNAP. We transformed these cells with the plasmid pRT19 encoding an arabinose inducible dCas9, which upon addition of arabinose represses the synthetic replication operon, and a plasmid encoding the terminal protein and a DNA polymerase of interest (plasmid pRT3 for WT, pRT4-2 for N71D mutant and pRT4-9 for Y127A mutant) under the control of a rhamnose-inducible promoter. The two strains were inoculated into 1 ml 2XTY media with kanamycin, tetracycline and apramycin, and grown at 37° C. while shaking (220 rpm) for 12 h. Then 1 μL of the culture was inoculated into 1 mL 2XTY media with kanamycin, tetracycline, apramycin and 10 mM rhamnose, and grown at 37° C. while shaking (220 rpm) for 12 h (~2¹⁰, 10 generations). All samples were then diluted and plated on plates with 150 μg ml⁻¹ chloramphenicol. 48 colonies for each mutant (N71D and Y127A) were Sanger sequenced. The transition (G:C to A:T) rate was calculated from the ratio of transition (G:C to A:T) to transversion (G:C to C:G). (FIG. 18B).

### Example 12—Proof of Evolving Concept

As proof-of-principle, the utility of this system may be used to direct evolution of a strong promoter from a weak promoter.

### Example 13—Accelerated Continuous Evolution of Tigecycline Resistance

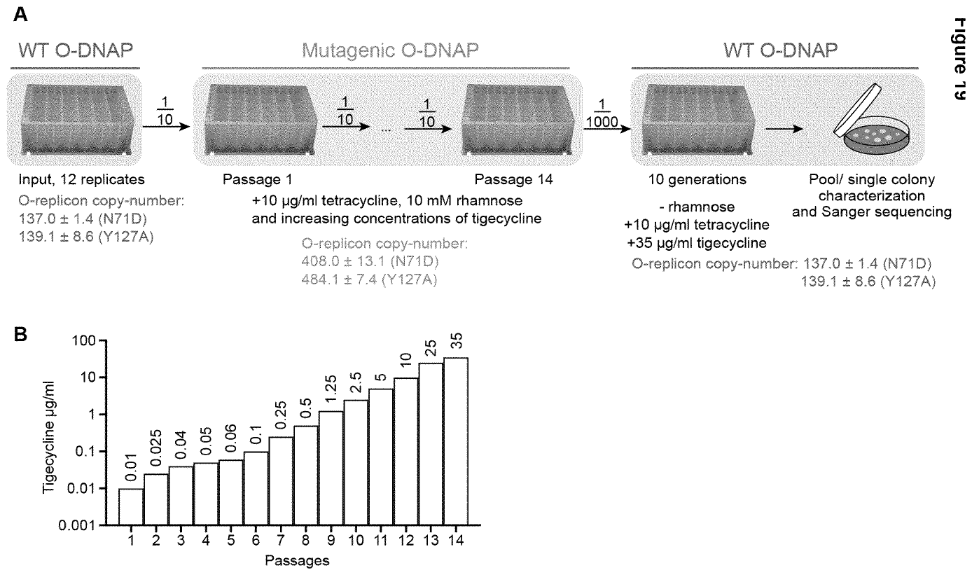

Next, we asked whether we could use the orthogonal replication system to continuously evolve new function. We first investigated converting the tetracycline resistance gene, tetA, into a gene that confers resistance to tigecycline. We created a Kan^{R}-TetA orthogonal replicon and transformed this into E. coli bearing a synthetic replication operon. To initiate mutagenesis of the replicon, we induced expression of a plasmid encoded O-DNAP via addition of rhamnose. We grew cells in kanamycin, apramycin (to maintain the O-DNAP-encoding plasmid), rhamnose, and tetracycline (10 μg ml⁻¹), with increasing concentrations of tigecycline (from 0.01 μg ml⁻¹ to 35 μg ml⁻¹, FIG. 19). We passaged cells once they had reached OD₆₀₀>0.5 and diluted them 1:10 in fresh media with a higher level of tigecycline. We completed 14 passages in 12 days. We performed 12 replicates with O-DNAP (N71D) and 12 replicates with O-DNAP (Y127A), with similar results.

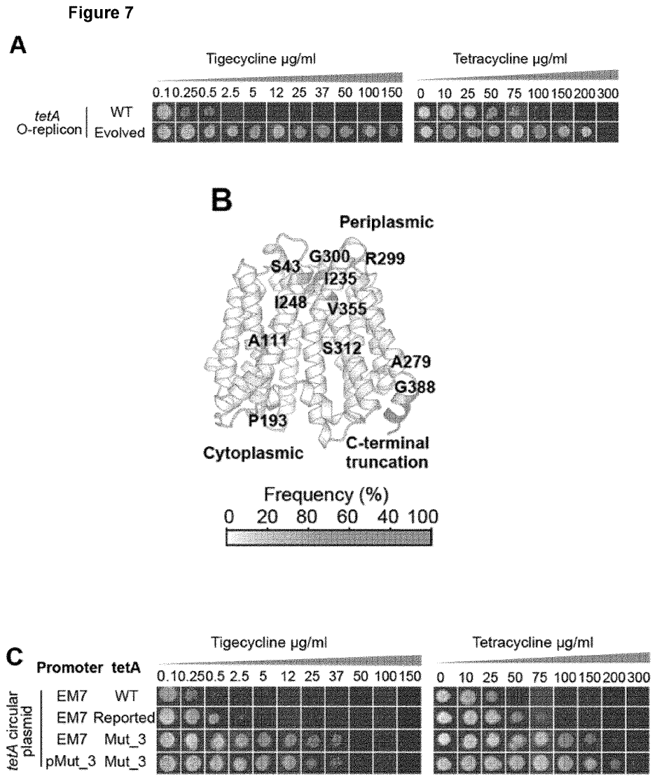

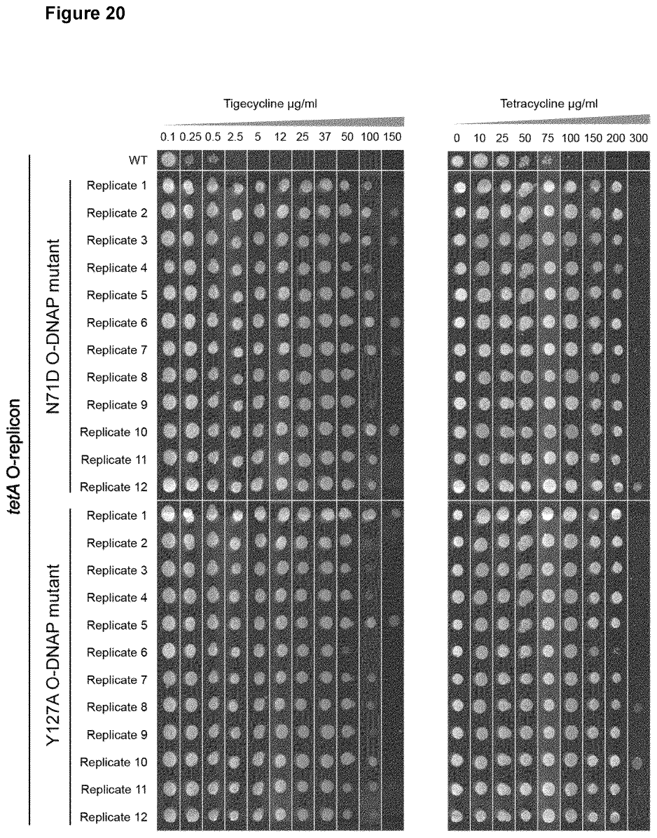

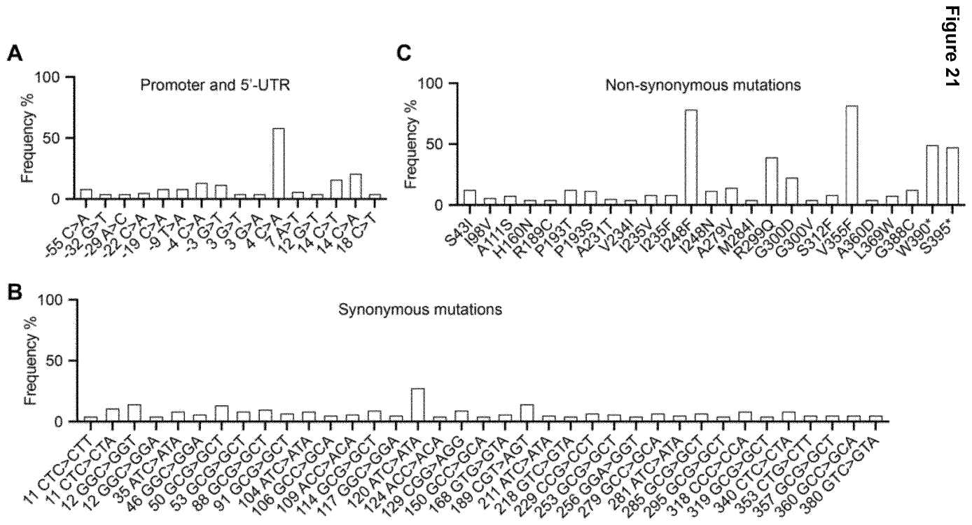

Cells containing the WT tetA gene on the orthogonal replicon grew on agar plates containing tigecycline at 0.5 μg ml⁻¹, but failed to grow on 2.5 μg ml⁻¹ tigecycline. However, after fourteen passages we obtained a pool of cells that grew on 150 μg ml⁻¹ tigecycline (FIG. 7A, FIG. 20). We sequenced five colonies from each of the 12 replicates performed with each of the two O-DNAPs (120 total colonies) and identified numerous mutations across the promoter and 5′UTR, as well as synonymous and non-synonymous mutations in the open reading frame (FIG. 20). Our experiment directly identifies mutations in tetA that have previously been implicated in tigecycline resistance, as well as a series of new mutations (FIG. 7B, FIG. 21). In contrast to a previous evolution, where enhanced tigecycline resistance culminated in reduced tetracycline resistance (10), we increased tolerance to both tigecycline and tetracycline simultaneously in all replicates (FIG. 20). Notable mutations include: the highly frequent 1248F (78.3%) and V355F (81.7%) mutations; the mutually exclusive, adjacent R299Q (39.2%) and G300D (22.5%) mutations; and C-terminal truncations which were found in 96.7% of sequenced clones across the 12 replicates for each O-DNAP (either W390* at a frequency of 49.2%, or S395* at a frequency of 47.5%).

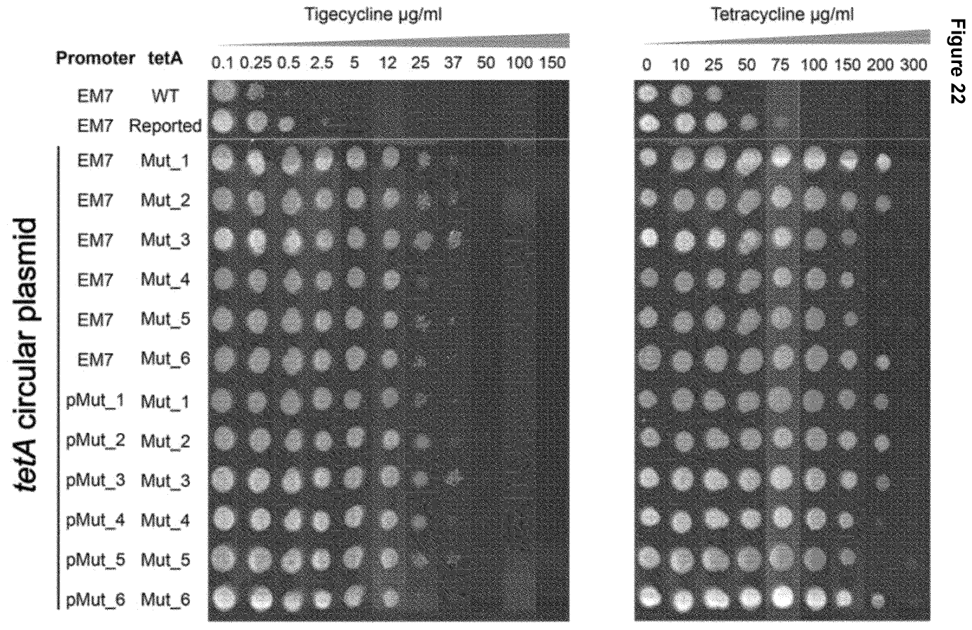

We picked colonies that grew on 150 μg ml⁻¹ tigecycline and cloned the genes into a standard circular plasmid (ColE1). The evolved tetA gene conferred tigecycline resistance to 37 μg ml⁻¹, whereas the parent tetA gene conferred resistance to 0.25 μg ml⁻¹ and a previously reported tetA gene for tigecycline resistance conferred resistance to 0.5 μg ml⁻¹ (FIG. 7C, FIG. 22). We compared the performance of our evolved tetA genes under the control of their evolved promoters or under the control of EM7. This revealed the contributions from the evolved promoters to be negligible. We thus conclude that we have evolved a tigecycline resistance gene that confers resistance to 150 times the level of the starting gene and 74 times the level of previously reported tigecycline resistance genes, in 12 days.

### Example 14—Accelerated Continuous Evolution of GFP Fluorescence

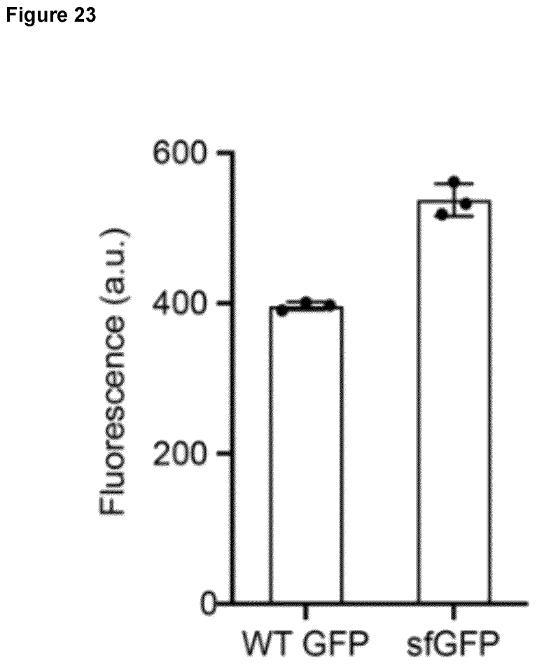

Next, we aimed to continuously evolve a GFP gene for increased fluorescence. We created a Kan^{R}-GFP orthogonal replicon, in which GFP is under control of a weak PJ23117 promoter. We started our continuous evolution with a variant of sfGFP, where the Thr₆₆-Tyr₆₇-Gly₆₈ chromophore was substituted to His₆₆-Tyr₆₇-Gly₆₈; this chromophore variant is found in the photoconvertible protein Kaedae (27), and the protein exhibits 73% of the sfGFP signal when expressed in cells (FIG. 23). For simplicity, we refer to our starting variant as ‘WT GFP’. We transformed this orthogonal replicon into E. coli bearing the synthetic replication operon. We switched to replicating the orthogonal replicon using the plasmid-encoded O-DNAP of interest (O-DNAP (N71D) or O-DNAP (Y127A), 12 replicates for each), as described above.

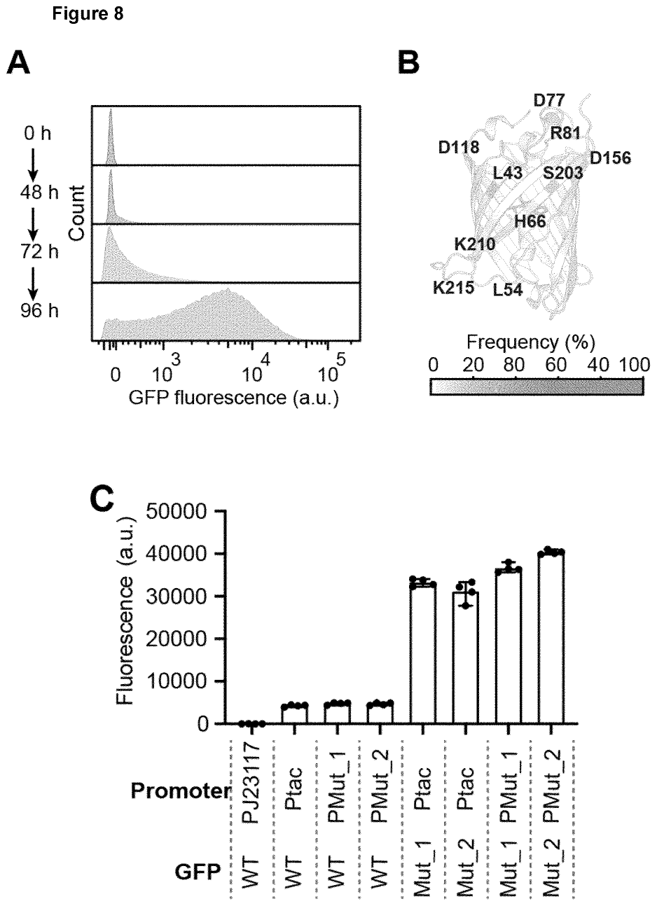

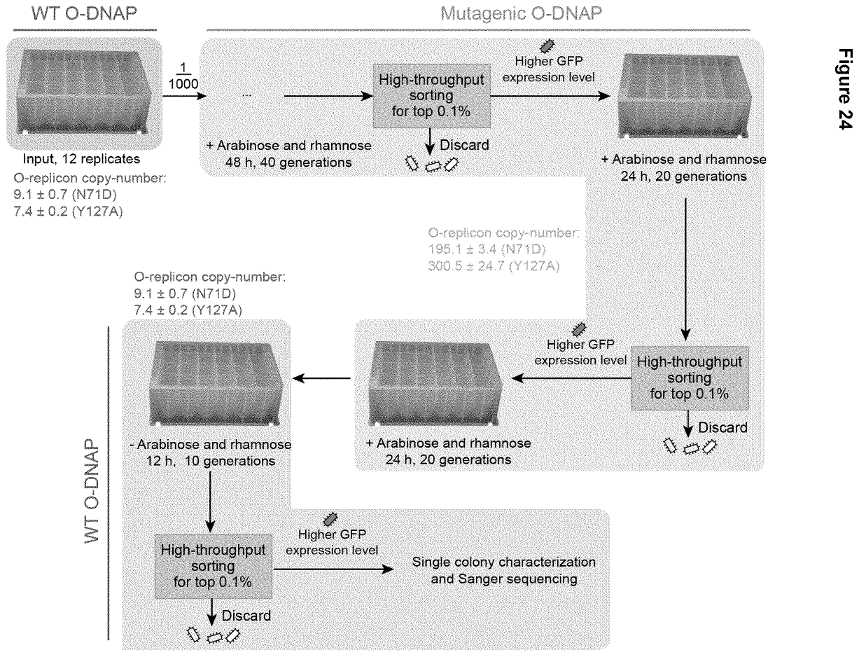

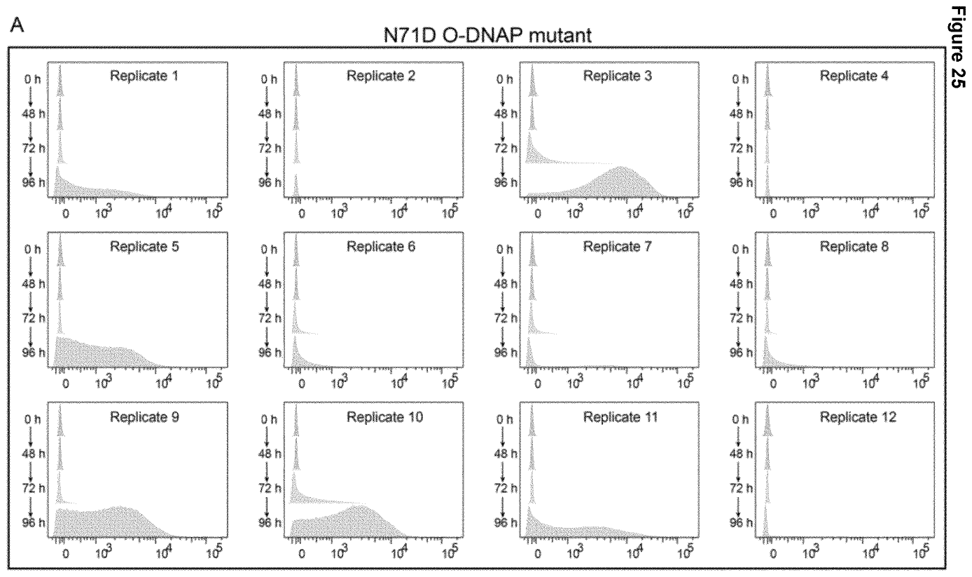

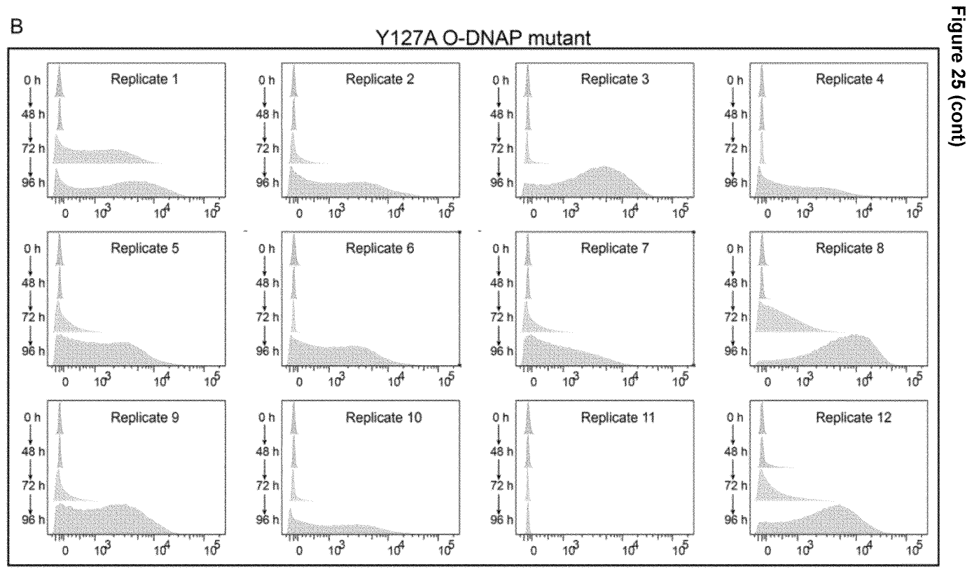

The original KanI-GFP orthogonal replicon exhibited weak fluorescence (FIG. 8A). Cells were diluted 1,000-fold from a saturated culture and grown for 12 h before 1,000-fold dilution into fresh media. This process was repeated four times, over 48 h, before cells were sorted for GFP fluorescence. The resulting cells were then grown for a further 48 h, with sorting for GFP fluorescence at 24 and 48 h; we performed all 12 replicates of this experiment for each of the two O-DNAPs in less than five days (FIG. 24). The population of cells progressively increased in fluorescence over the course of the experiment (FIG. 25).

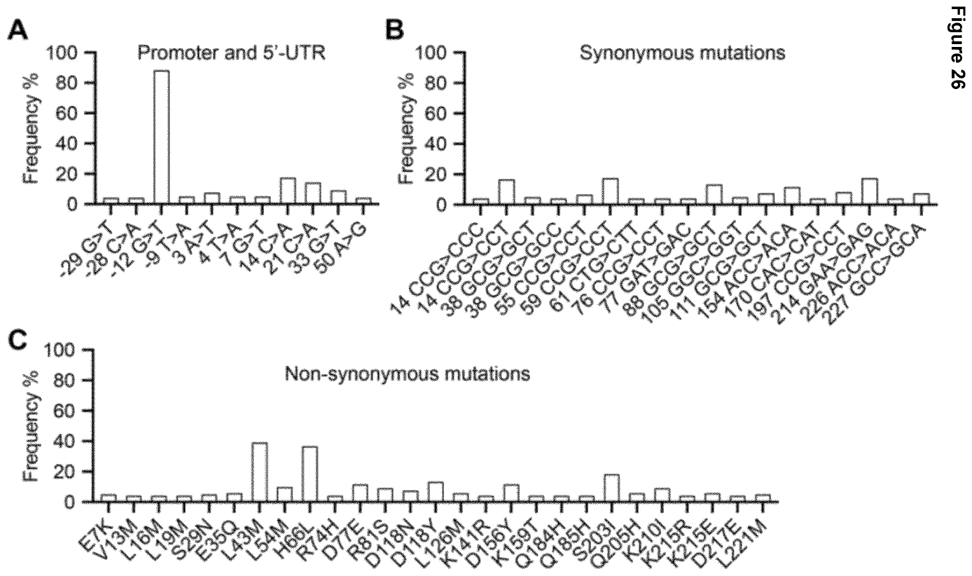

We sequenced five colonies from each of the 12 replicates performed with each of the two O-DNAP mutants (120 colonies total) and identified numerous mutations across the promoter and 5′UTR, as well as synonymous and non-synonymous mutations in the open reading frame (FIG. 8B, FIG. 26). Our experiment directly identifies mutations in the promoter that convert the −10 sequence to a consensus sequence, and we identify a number of enriched mutations in the coding sequence (FIG. 26). The two most frequent mutations were L43M (39.2%) and H66L (36.7%), and the occurrence of these mutations was mutually exclusive. Whereas the H66L mutation directly converts the chromophore to Leu₆₆-Tyr₆₇-Gly₆₈, which has previously been described to display four- to sixfold greater green fluorescence than the canonical GFP chromophore (Ser₆₆-Tyr₆₇-Gly₆₈) (28), the alternative L43M mutation has not been described and appears to be compensatory—the Leu₄₃ sidechain points into the beta barrel, towards the chromophore, and thus its substitution to Met might enable interactions that could culminate in improved chromophore maturation or stability.

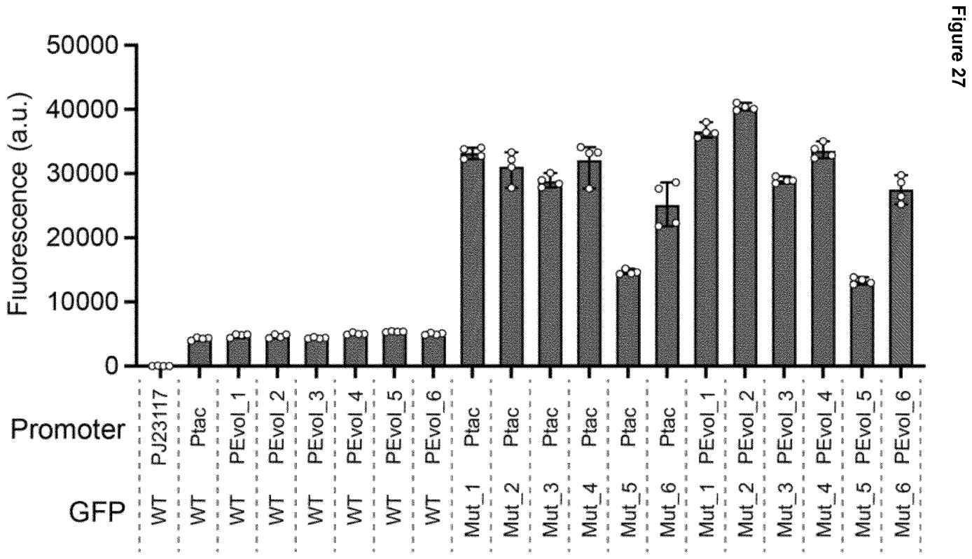

We picked colonies that exhibited strong fluorescence and cloned the corresponding gene into a standard circular plasmid (ColE1). The selected constructs (pEvol 1/GFP Mut_1 and pEvol_2/GFP Mut_2) produced 36,586±874 and 40335±442 au of fluorescence, while the WT GFP gene on a PJ23117 promoter produced 33±24 au of fluorescence (FIG. 8C, FIG. 27). Thus, selection using the orthogonal replication system increased the green fluorescence by more than 10³-fold in less than five days.

To investigate the contributions of selected promoters (PEvol_1 and PEvol_2) and the selected open reading frames (GFP Mut_1 and GFP Mut_2) to the observed increase in fluorescence, we expressed the WT GFP gene 50 from PEvol_1 and PEvol_2. PEvol_1 and PEvol_2 both contain a transversion mutation in the −10 sequence that converts the −12 base from G to T, generating a consensus −10 promoter sequence. PEvol_1 and PEvol_2 also both mutated position 14 in the 5′ UTR—PEvol_1 underwent a C to T transition, whereas in PEvol_2 this base mutated to A (transversion). GFP Mut_1 contains the H66L mutation (and D77E, D118N, S2031) and GFP Mut_2 contains the L43M mutation (and R81S, K215E).

PEvol_1/WT GFP and PEvol_2/WT GFP led to 4769±226 and 4679±262 au of fluorescence (FIG. 8C). For comparison, the WT GFP gene on a strong Ptac promoter produced 4256±200 au of fluorescence. We conclude that the selected promoters are approximately 144-fold stronger than the promoter we started with, and are comparable in strength to a strong Ptac promoter.

Comparing the fluorescence of GFP Mut_1 and GFP Mut_2 to WT-GFP, when each GFP variant is expressed from the same promoter (PEvol_, PEvol_2 or Ptac, (FIG. 8C)), suggests that the evolved coding sequences produce approximately nine times more fluorescence than the starting WT-GFP, and seven times more fluorescence than sfGFP. Thus, mutations both the promoter and the open reading frame of GFP make substantial contributions to the observed increase in cellular fluorescence.

### Example 15—Discussion

We have booted up an orthogonal replicon in a living organism for the first time; we achieved this in E. coli, the most widely used and best characterized host, by endowing cells with a rationally designed synthetic replication operon. Our work demonstrates that orthogonal replication systems can be created de novo, to enable the generation of mutagenic continuous cellular evolution systems in organisms beyond the extremely limited set where natural replicons exist and can be modified in vivo (12-14). The orthogonal linear double stranded DNA replicon simply requires 18 bp DNA sequences at each end and can carry diverse cargos, including cargos too large for viral systems. The dynamic range of our control over replicon copy number exceeds that of control systems for circular plasmid copy number (29, 30).

Our rationally designed mutant O-DNAPs enable continuous mutagenic derivatization of user-defined genes within the orthogonal replicon. These polymerases continually mutate the orthogonal replicon at a mutation rate above the critical error threshold for the E. coli genome and even above the rates achieved by toxic, unsustainable global mutagenesis methods (26). EcORep therefore provides a powerful method for massively accelerated continuous evolution without restricting evolutionary outcomes through mutational bias. We thus expect EcORep to have immediate utility as a streamlined, widely accessible platform for continuous evolution experiments in E. coli, thereby overcoming the need for low-throughput DNA transformation and extraction steps, targeted in vivo mutagenesis strategies with limited mutational windows, or custom setups for linking propagation of gene variants to the selection for a desired phenotype.

Given the extensive collection of genetic or cell-based selections available for E. coli, EcORep should facilitate the continuous evolution of genes, including entire genetic pathways, of highly diverse function. For example, EcORep will readily integrate with: FACS-based selections, as we demonstrated for GFP evolution; antibiotic-driven evolutions to discover the emergence of clinically-relevant mutations for antibiotic resistance (31), as we demonstrate for tetA; high-throughput droplet screening approaches for enzyme evolution (32); surface display and capture strategies to enrich antibody fragments of biopharmaceutical value (33); bacterial two-hybrid screens to generate new protein-protein interactions or inhibitors thereof (34, 35); and approaches that couple a desired phenotype to improved growth (14). As EcORep avoids the restrictions of library size imposed by in vitro library preparation, the approach is inherently scalable; continuous evolution experiments can be run via simple serial passaging, or at bioreactor scale. Experiments with ever expanding library sizes will enable studies of extreme genetic drift; and high-throughput parallelized evolutions will facilitate mapping evolutionary trajectories and dynamics, or the selection for multiple phenotypes simultaneously.

EcORep provides a simple, stable, and scalable platform for accelerated continuous evolution in E. coli. We anticipate that it will substantially accelerate the development of diverse research tools, biopharmaceutical leads, and strains for the production of industrial chemicals.

### Materials and Methods

### Strains

All strains used in this study are derived from E. coli DH10b. We integrated the synthetic replication operon under the control of different promoters into the genome of E. coli through CRISPR-assisted lambda red knock-in, as described before (1).

### Construction of Orthogonal Replicons

Linear replicons used for electroporation were obtained by overlap extension PCR (PrimeSTAR Max DNA Polymerase, TAKARA, Tokyo, Japan). Specifically, four DNA fragments with overlaps, including left replication origin, antibiotic resistant genes, genes of interest and right replication origin are first obtained by PCR and gel purified. Then a single primer (with or without 5′-phosphorylation; we initially used phosphorylated primers as 5′-end phosphorylation had been shown to improve replication of an in vitro reconstituted phi29 replication system which also operates via protein-primed replication, but this is not the case for EcORep) was used to amplify the whole length orthogonal replicon by binding to both inverted repeat ends of the template. The antibiotic genes used for the orthogonal replicons, and the concentrations of the corresponding antibiotic used for selections include: kanamycin resistance gene (Kan^{R}, 50 μg mL⁻¹), chloramphenicol resistance gene (Cm^{R}, 20 μg mL⁻¹), tetracycline resistance gene (tetA, 10 μg mL⁻¹), apramycin resistance gene (Apn, 50 μg mL⁻¹) and ampicillin resistance gene (Amp^{R}, 50 μg mL⁻¹).

Construction of circular plasmids To increase the orthogonal replicon transformation efficiency, three helper plasmids, pFR160 (encoding gam), pFR160SB (encoding SSB and DSB) and pFR160 GB (encoding gam, SSB and DSB) were designed based on the CloDF13 replication origin, a gentamicin resistant gene (Gen^{R}) and streptomycin sensitivity negative selection marker (rpsL).

To test the essentiality of all 4 genes encoded by the synthetic replication operon, we designed single-copy plasmids bearing the intact synthetic replication operon (plasmid pRT9) or operons in which each of the four genes in the operon (encoding the terminal protein (plasmid pRT9-dTP), DNA polymerase (plasmid pRT9-dDNAP), single strand binding protein (plasmid pRT9-dSSB) and double strand binding protein (plasmid pRT9-dDSB)) were disrupted. All plasmids were designed based on a bacterial F plasmid replication origin, a chloramphenicol resistant gene (Cm^{R}) and sucrose sensitivity negative selection marker (sacB).

To control the orthogonal replicon copy number, plasmids for downregulating expression of the synthetic replication operon via an arabinose inducible dCas9 targeted to the IPTG-responsive Ptac promoter were designed. We designed the 3 plasmids encoding dCas9 and 3 different sgRNA (named pRT17, pRT19 and pRT21, respectively) based on CloDF13 replication origin, tetracycline resistant gene (tetA) and streptomycin sensitivity negative selection marker (rpsL).

Plasmids overexpressing TP (plasmid pRT1) or DNAP (plasmid pRT2), or a combination of both (plasmid pRT3) under the control of a rhamnose inducible promoter were designed based on a p15A replication origin and an apramycin resistant gene (apmR). We also designed plasmids with the same architectures to over express TP with all other ODNAP mutants (pRT4-1 for T20I, pRT4-2 for N71D, pRT4-3 for N71D and L341R, pRT4-4 for H70R, pRT4-5 for D17A, pRT4-6 for D76A, pRT4-7 for D17A and D76A, pRT4-8 for D17A, D76A and L341R, pRT4-9 for Y127A). The plasmid pRT4-0 is a plasmid with the same architecture but does not express TP nor DNAP.

All circular plasmids were constructed using HiFi Gibson Assembly (New England Biolabs, Massachusetts, United States) from multiple fragments.

### Electroporation Protocol for Booting Up Orthogonal Replicons

First, colonies harboring the helper plasmid pFR160 GB and a genomically-integrated or plasmid-encoded synthetic replication operon were inoculated into 15 ml 2XTY media and grow at 37° C. while shaking (220 rpm) for 12 h. Second, 5 mL of the resulting culture was used to inoculate a 2 L shake flask containing 200 mL 2XTY media and grown at 37° C. while shaking (220 rpm) to and OD₆₀₀ of ~0.2. Arabinose was then added to the culture to a final concentration of 10 mM. Once cells reached an OD₆₀₀ of ~0.4, they were chilled on ice while shaking (100 rpm) for 10 min and then spun down (4500×g, 3 min, 4° C.) and washed in 50 mL pre-chilled 10% glycerol twice. After washing, cell pellets were finally resuspended in 1000 μL of ice-cold 10% glycerol and aliquots of 100 μL were used for orthogonal replicon electroporation. For electroporation, 6 μL purified orthogonal replicon PCR product (around 600 ng μL⁻¹, in water) was mixed with 100 μL competent cells. The mixture was added to an ice-cold electroporation cuvette (2 mm gap; SLS scientific), and incubated on ice for 15 min. The electroporation was performed using Eppendorf e-porator (2500 V) and 1 mL of pre-warmed SOB media was then immediately added to the cuvette. After incubating the mixture at 37° C. while shaking (220 rpm) for 2 h, all cells were plated on an agar plate with corresponding antibiotics.

### Orthogonal Replicon Extraction

After we established a Kan^{R}-GFP orthogonal replicon composed of a kanamycin resistance gene and a GFP gene (Tac promoter and T7 terminator for GFP, native promoter for Kan^{R}), flanked by 110 bp PRD1 inverted terminal repeat sequences on each end in vivo, we inoculated the cells into 5 mL 2XTY media and cultured them at 37° C. while shaking (220 rpm) for 12 h. Then 1 mL of the culture was further inoculated into a 500 mL shake flask containing 50 mL 2XTY media and grown at 37° C. while shaking (220 rpm) to and OD₆₀₀ of ~1.0.05 mM IPTG was then added to the culture and the cells were further incubated until an OD₆₀₀ of ~3. To extract the orthogonal replicon from the cells, we lysed the cells and performed a standard plasmid extraction protocol using QIAprep Spin Miniprep Kit (QIAGEN, Hilden, Germany). We added 50 μg mL⁻¹ proteinase K to buffer II of the kit to remove the covalently bound terminal protein from the ends of the orthogonal replicon, whereas for the control we did not add proteinase K. Before running the extracted replicon on an agarose gel, we added 50 mM NaCl and re-annealed the replicon in a thermocycler over a 1 h gradient from 98 to 12° C. The linear replicons extracted without proteinase K addition could also be directly transformed into cells with the synthetic replication operon.

### Orthogonal Replicon Stability Test

To test the stability of the orthogonal replicon, the cells were inoculated into a 96-well plate containing 200 μL 2XTY, with corresponding antibiotics, per well and grown at 37° C. with shaking (750 rpm) for 12 h. The culture, regarded as generation 0, was then inoculated into the next well containing 200 μL 2XTY, with or without the corresponding antibiotics, at a dilution factor of 1/1000 (10 generations) and cultured for 12 h (~2¹⁰-fold increase in cells, generation 10). The inoculation and culturing steps were repeated 10 times, until generation 100 or 300. All the intermediate samples were analyzed by flow cytometry using a BD LSRFortessa Cell Analyzer (Becton Dickinson, New Jersey, United States) to determine the fluorescence of single cells. Samples were manually gated for single bacterial cells. Autofluorescent cells were discarded from analysis. Gating of green fluorescent cells was set relative to the negative control not expressing GFP. The gating strategy is also shown in FIG. 11. The orthogonal replicon stability was calculated based on the proportion of single GFP-fluorescing cells.

### GFP Expression Measurements

To test the GFP expression levels of cells harboring orthogonal replicons or circular plasmids, we first inoculated all strains into 96-well plates containing 200 μL 2XTY media with the corresponding antibiotics per well and grew them at 37° C. while shaking (750 rpm) for 12 h. Then we inoculated 10 μL of all the cultures into 190 μL 2XTY media with all corresponding antibiotics and the different concentrations of inducers. The plates were incubated at 37° C. while shaking (750 rpm) for 12 h, and we took aliquots to determine plasmid copy number via qPCR (below). After that, we diluted 50 μL of the samples to 100 μL of PBS in 96-well flat-bottom clear plates, and measured GFP fluorescence (λₑₓ: 485 nm; λₑₘ: 520 nm) and OD₆₀₀ using a PHERAstar FS plate reader (BMG Labtech, Ortenberg, Germany). In all cases, we report the GFP fluorescence normalized by OD₆₀₀.

### Plasmid Copy Number Assay

For all samples, we first spun down and resuspended all cells in water and adjusted them to OD₆₀₀~1. Then a lysis buffer (QuickExtract DNA Extraction Solution) was used to lyse all cells, and we used the lysate as template for quantitative real-time PCR (qPCR) to test target DNA copy number. The reference gene was the dxs gene on the genome, as it has been shown to be a stable one-copy reference in E. coli. The target gene used was kanR. We used a fused DNA template containing the dxs gene and kanR gene to make sure that the copy number of the two genes was equal. We used qPCR to determine the DNA copy numbers for all samples with a Vii 7 Real-Time PCR System with 384-Well Block (Thermo Fisher, Massachusetts, United States).

Next-generation sequencing and analysis After we obtained the colonies containing the 16.5 kb orthogonal replicon, we amplified it by colony PCR (PrimeSTAR GXL DNA Polymerase, TAKARA, Tokyo, Japan) and purified it using a DNA Purification kit (QIAGEN, Hilden, Germany). Samples were then paired-end sequenced on the NextSeq 2000 (Illumina, P1 reagent kit v3 (300 cycles). The raw reads from the NextSeq2000 were then aligned against the reference FASTA file and read depth was analyzed as described before (https://github.com/JWChin-Lab/NGS-analysis).

### Mutation Rate Measurements

We used Luria-Delbrack fluctuation analysis to measure the genomic mutation rate of all strains. First, we introduced a Cm^{R}(Q38TAG) gene that contains an amber stop codon (TAG) at position 38 into the genome of E. coli DH10b. The insertion site was adjacent to the lacI gene, distal from the origin of replication (2,049,646 bp from oriC), where the copy number is expected to be approximately one. We transformed this strain with pRT4-0 or the plasmid encoding each DNAP of interest (plasmid pRT3 for WT, pRT4-2 for N71D and pRT4-9 for Y127A) under the control of a rhamnose promoter. Point mutations that convert the TAG stop codon to sense codons can confer chloramphenicol resistance. Second, we inoculated strains into 1 mL 2XTY media and grew them at 37° C. while shaking (220 rpm) for 12 h. Then 1 μL of the culture was inoculated into 1 mL 2XTY media supplemented with apramycin and 10 mM rhamnose, and the cultures were grown at 37° C. while shaking (220 rpm) for 12 h (~2¹⁰-fold increase in cells after growth, 10 generations). All samples were then diluted and plated on plates with or without 20 μg ml⁻¹ chloramphenicol, to test the proportion of chloramphenicol-resistant cells. 12 biological replicates were performed for each strain. FALCOR was used to calculate mutation frequency (m) based on cell numbers on the selection and non-selection plates for all 12 biological replicates. Mutation per generation per base pair p (s.p.b.) was calculated using μ (s.p.b.)=m/(R×C). For the parameter R, which is the number of distinct mutation sites that make the resistance gene effective, we found that 8/9 possible single base substitutions (which yield sense codons) can result in chloramphenicol resistance, as determined by Sanger sequencing of resistant clones. So, R=8/3. C is the gene copy number and equals 1 because the gene is integrated at a genomic locus where the copy number is expected to be one.

To measure the mutation rate in replication of the orthogonal replicon, we created a Kan^{R}-Cm^{R}(Q38TAG) orthogonal replicon that contains an amber stop codon (TAG) at position 38 of the chloramphenicol resistance gene. We introduced this orthogonal replicon into cells containing a synthetic replication operon with a WT DNAP. We transformed these cells with the plasmid pRT19 encoding an arabinose inducible dCas9, which upon addition of arabinose represses the synthetic replication operon, and a plasmid encoding terminal protein and a DNA polymerase of interest (plasmid pRT3 for WT, pRT4-1 to pRT4-9 for all mutants) under the control of a rhamnose promoter. All strains were inoculated into 1 ml 2XTY media with kanamycin, tetracycline and apramycin, and grown at 37° C. while shaking (220 rpm) for 12 h. Then 1 μL of the culture was inoculated into 1 mL 2XTY media with kanamycin, tetracycline, apramycin and 10 mM rhamnose, and grown at 37° C. while shaking (220 rpm) for 12 h (~2¹⁰, 10 generations). All samples were then diluted and plated on plates with or without 20 μg ml⁻¹ chloramphenicol, to test the proportion of chloramphenicol-resistant cells. For the first round of mutational measurement, 4 biological replicates were performed for each strain. Mutation frequency (m) was calculated by directly dividing the number of colonies on chloramphenicol resistance selection plates by the total number of cells. For the second round, precise mutation rate measurements of mutant N71D and Y127A, 12 biological replicates were performed and FALCOR was used to calculate the mutation frequency (m). Substitutions per bp per generation, p (s.p.b.), were calculated using p (s.p.b.)=m/(R×C), where R=8/3 and C=copy number of orthogonal replicons tested by qPCR for each sample.

To measure the mutation rate in replication of the orthogonal replicon when the cell only expresses one O-DNAP variant in the absence of the induction system described above, we transformed the helper plasmid pFR160 GB into 3 strains containing a genomically-integrated synthetic replication operon with a WT O-DNAP, mutant N71D and Y127A, respectively. We then electroporated the Kan^{R}_Cm^{R}(Q38TAG) orthogonal replicon into the 3 strains. After electroporation, 1.2 ml of the culture was incubated at 37° C. while shaking (220 rpm) for 3 h and divided into three aliquots (400 μL each). The first 400 μL of the culture was directly plated on plates with kanamycin and chloramphenicol to make sure no cells were chloramphenicol resistant at the very beginning. The second 400 μL was plated on plates with kanamycin to calculate the number of cells that have successfully established the Kan^{R}_Cm^{R}(Q38TAG) orthogonal replicon. The last 400 μL of the culture was inoculated to 2 mL 2XTY media with kanamycin and grown at 37° C. while shaking (220 rpm) for 12 h. All samples were then diluted and plated on plates with or without 20 μg ml⁻¹ chloramphenicol, to test the proportion of chloramphenicol-resistant cells. 12 biological replicates were performed and FALCOR was used to calculate the mutation frequency (m). The generations were calculated from the initial and final cell numbers.

### Mutation Spectrum Measurements

We Sanger sequenced 96 colonies from plates (with 20 μg ml⁻¹ chloramphenicol) from the mutation rate measurement experiments for mutant N71D and Y127A, and calculated the mutational spectrum of transition (A:T to G:C) and transversion (A:T to T:A, A:T to C:G, G:C to T:A, G:C to C:G). To measure the transition (G:C to A:T) data, we designed a Cm^{R}(S145G) orthogonal replicon, a single base pair mutation from G (GGT) to S (AGT) or R (CGT) can confer resistance to 150 μg ml⁻¹ chloramphenicol. We introduced this orthogonal replicon into cells containing a synthetic replication operon with a WT DNAP. We transformed these cells with the plasmid pRT19 encoding an arabinose inducible dCas9, which upon addition of arabinose represses the synthetic replication operon, and a plasmid encoding the terminal protein and a DNA polymerase of interest (plasmid pRT3 for WT, pRT4-2 for N71D mutant and pRT4-9 for Y127A mutant) under the control of a rhamnose-inducible promoter. The two strains were inoculated into 1 ml 2XTY media with kanamycin, tetracycline and apramycin, and grown at 37° C. while shaking (220 rpm) for 12 h. Then 1 μL of the culture was inoculated into 1 mL 2XTY media with kanamycin, tetracycline, apramycin and 10 mM rhamnose, and grown at 37° C. while shaking (220 rpm) for 12 h (~2¹⁰, 10 generations). All samples were then diluted and plated on plates with 150 μg ml⁻¹ chloramphenicol. 48 colonies for each mutant (N71D and Y127A) were Sanger sequenced. The transition (G:C to A:T) rate was calculated from the ratio of transition (G:C to A:T) to transversion (G:C to C:G).

### AlphaFold2 Structure Prediction

The PRD1 PolB structure and TetA structure were predicted using ColabFold version of AlphaFold2 algorithm implemented in ChimeraX (v1.5). The structures were visualized in PyMOL (v2.5.4).

Accelerated Continuous Evolution of tetA

We first created a Kan^{R}-TetA orthogonal replicon and transformed this into E. coli bearing a synthetic replication operon, cured the helper plasmid (pFR160 GB), and then transformed the strain with a plasmid encoding an O-DNAP of interest (pRT4-2 for N71D and pRT4-9 for Y127A) under the control of a rhamnose promoter. For the continuous evolution experiment, we performed 12 replicates with O-DNAP (N71D) and 12 replicates with O-DNAP (Y127A). We inoculated all strains into a 24-well plate containing 1 mL 2XTY with kanamycin, tetracycline and apramycin per well and cultured it at 37° C. while shaking (220 rpm) for 12 h. Then, 100 μL of the culture was inoculated into 1 mL 2XTY media with kanamycin, tetracycline, apramycin and 10 mM rhamnose, and grown at 37° C. while shaking (220 rpm). We passaged cells once all 24 wells had reached OD₆₀₀>0.5 and diluted them 1:10 in 1 mL fresh 2XTY medium containing kanamycin, tetracycline, apramycin, 10 mM rhamnose and increasing concentrations of tigecycline (from 0.01 μg mL⁻¹ to 35 μg mL⁻¹). We completed 14 passages in 12 days. After the final passage under continuous mutagenesis, we passaged cells into rhamnose-free medium to switch to using the WT O-DNAP, thereby switching off further mutagenesis. After plating the resultant cultures on agar plates containing 35 μg mL⁻¹ tigecycline, we sequenced individual colonies by Sanger sequencing of colony PCR products. Select variants were then cloned into a ColE1 plasmid backbone for validation.

### Accelerated Continuous Evolution of GFP

We created a Kan^{R}-GFP orthogonal replicon, in which GFP (T66H mutation in the chromophore, exhibits 73% of the sfGFP signal when expressed in cells) is under control of a weak PJ32117 promoter. We transformed this orthogonal replicon into E. coli bearing the synthetic replication operon and transformed this strain with the plasmid pRT19 encoding an arabinose inducible dCas9, which upon addition of arabinose represses the synthetic replication operon, and transformed this strain with a plasmid encoding a O-DNAP of interest (pRT4-2 for N71D and pRT4-9 for Y127A) under the control of a rhamnose promoter. For the continuous evolution experiment, we performed 12 replicates with O-DNAP (N71D) and 12 replicates with O-DNAP (Y127A). We inoculated all strains into a 24-well plate containing 1 mL 2XTY (containing kanamycin, tetracycline and apramycin) per well and grew the cultures at 37° C. while shaking (220 rpm) for 12 h. Then 1 μL of the cultures was inoculated into 1 mL 2XTY media (diluted 1,000-fold) supplemented with kanamycin, tetracycline, apramycin, 1 mM arabinose and 10 mM rhamnose, and grown at 37° C. while shaking (220 rpm) for 12 h. In this process, cells were diluted 1,000-fold from a saturated culture and grown for 12 h before 1,000-fold dilution into fresh 2XTY media. This process was repeated four times, over 48 h, before cells were sorted for GFP fluorescence. The Bigfoot Spectral Cell Sorter (Thermo Fisher, Massachusetts, United States) was used to sort for cells with the top 0.1% highest fluorescence intensities. The resulting cells were then grown for a further 48 h, sorting for GFP fluorescence at 24 and 48 h. Before the last sorting step, we inoculated 1 μL of all samples into a 24-well plate containing 1 mL 2XTY (with kanamycin, tetracycline and apramycin) per well and grew the cultures at 37° C. while shaking (220 rpm) for 12 h, to switch from the plasmid encoded O-DNAP mutants to the genomically encoded WT O-DNAP. After the final selection, cells were plated and individual colonies were sequenced via Sanger sequencing of colony PCR products. Select variants were then cloned into a ColE1 plasmid backbone for validation.

### Example 16—Linear Replication Establishment by In Vivo Cleavage of Circular DNA

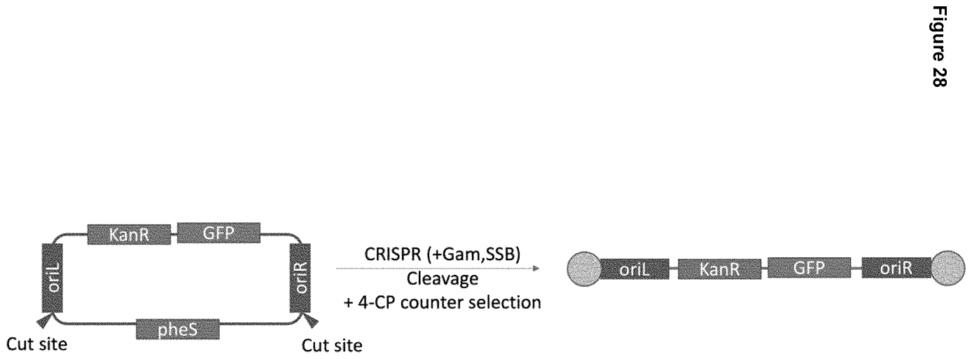

The present researchers have demonstrated that EcORep can be established by double cleavage of a circular plasmid carrying the two origin of replication sequences. Cutting sites have been introduced outside the left and right origins of replications (see FIG. 28) meaning that once the circular plasmid is cut, it will no longer use the circular replication mechanism but, instead, initiates orthogonal linear replication by EcORep. With this approach, suitable circular episomes can be converted into the linear replication system to further enable continuous evolution of the replicon.

The advantage of this approach is that whilst the size of a PCR product for EcORep can be limited by current available commercial DNA polymerases, the size of a circular plasmid can be constructed up to hundreds of kilo base pairs in size. Hence enabling orthogonal linear replication of large DNA circuits, whilst applying the approach for the direct evolution of larger and more complicated metabolic pathways using the EcORep system.

### Materials

Strain: ERP3 strain
Scissorplasmid: CloDF13-tet^{R}-pBAD_Gam_Cas9_SSB_DSB-prhaB_sgRNA1,2
Target plasmid: pSC101-pheS*(T251A/A294G)-oriL-Kan^{R}-GFP-oriR
Inducers: 10 mM arabinose and 1 mM rhamnose
Antibiotics: 10 ug/ml tetracycline and 50 ug/ml kanamycin
Counter selection substrate: 2.5 mM 4-Chloro-DL-phenylalanine(4-CP)

### Methods

A scissor plasmid was constructed by Gibson assembly. A target plasmid was constructed by Gibson assembly. E.coli DH10B strain with genomic integrated PRD1 linear replication gene cluster, referred to as ERP3 strain, was prepared as electrocompetent cells and electroporated with the Scissor plasmid of tetracycline resistance. After recovery and growth on 2XTY tetracycline agar plate, a single colony was picked and inoculated for overnight culture. The overnight culture was 1:100 diluted to fresh 2XTY media with tetracycline. When the OD₆₀₀ of the culture reach to 0.2, arabinose and rhamnose were added to induce the expression of Cas9 and sgRNAs for cleavage, plus the Gam protein to inhibit recBCD nucleases system and SSB+DSB for DNA protection. When OD₆₀₀ of the culture reaches to 0.4-0.5, electrocompetent cells were prepared and 500 ng target plasmid were 50 electroporated (can be scaled up). After recovering in SOB media for 3 h, cells were plated on 2XTY agar plates with tetracycline, kanamycin and with 4-CP as tests or without 4-CP as control. Single colonies were genotyped by PCR amplification of the Kan^{R}-GFP region and a junction region covering upstream and downstream of the oriL. The plasmids of genotype correct clones were further extracted and sequenced by Illumina NextSeq for final confirmation.

### Result

After Cas9 cleavage and counter selection, we are expecting colonies growing on the 4-CP plates should only carry the linear plasmid instead of the circular plasmid. In addition, if the linear replication is successfully established, the copy number of the plasmids will increase from 3-4 per cell to around 70 per cell, resulting in a stronger fluorescent signal. Indeed, we observed colonies with stronger fluorescent signal compared to those growing without 4-CP counter selection. The plasmids from the clones were further extracted and sequenced by next generation sequencing (NGS). The NGS result shows that only the linear replicon containing the left and right origin, Kan^{R} and GFP gene were present, which demonstrate successful transformation from circular plasmid to linear plasmid after the cleavage.

### Example 17—O-Replicon Electroporation

After establishing the replication system in the E. coli cell, the o-replicon (the linear plasmid) can be extracted from the living cells using a standard miniprep protocol (QIAprep Spin Miniprep Kit, QIAGEN) without proteinase K treatment. Thus, the terminal proteins are already bound to the replicon ends enabling it to be efficiently transformed back into new E. coli cells containing a synthetic replication operon by electroporation (more than 106 colonies per transformation) without the assistance of a helper plasmid.

### Example 18—Only TP and ODNAP are Necessary for the Maintenance of the o-Replicon

It is demonstrated herein that the ODNAP, TP, and SSB, but not DSB, are necessary for establishing the linear replicon. In this Example, these findings are extended to identify the essential proteins for maintaining the O-replicon. Using the method of Example 17, we electroporated the extracted O-replicon into cells containing a synthetic replication operon encoding only TP and ODNAP. The transformation efficiency was also very high demonstrating that only the TP and DNAP are necessary for the maintenance of the linear replicon.

### Example 19—New ODNAP Mutants with Higher Error-Rates

To obtain ODNAP mutants with higher mutation rates, we first designed three o-replicons for ODNAP library screening:
a. Kan^{R}_Cm^{R}(Q38TAG) o-replicon, that contains an amber stop codon (TAG) at position 38 of the chloramphenicol resistance gene (Cm^{R}). Chloramphenicol resistance can be conferred to the cell only when the TAG stop codon mutates to a sense codon.
b. Kan^{R}_Cm^{R}(Q38TAG K50TGA) o-replicon, that contains an amber stop codon (TAG) at position 38 of the chloramphenicol resistance gene (Cm^{R}) and an opal stop codon (TGA) at position 50. Chloramphenicol resistance can be conferred to the cell only when the 2 stop codons both mutate to sense codons.
c. Kan^{R}_Cm^{R}(H193Q) o-replicon, that contains an amber stop codon (TAG) at position 193 of the chloramphenicol resistance gene (Cm^{R}). Chloramphenicol resistance can be conferred to the cell only when the Q (glutamine, CAG) mutates to H (Histidine, CAC or CAT).

We first introduced these 3 o-replicons into cells containing a synthetic replication operon with a WT DNAP to get strains TC1, TC2, and TCB respectively. We then transformed these cells with a plasmid encoding an arabinose inducible dCas9, which upon addition of arabinose represses the synthetic replication operon, to get strains TC1-dCa9, TC2-dCas9, and TCB-dCas9.

### First Round of Screening

We generated an ODNAP random mutation library by error-prone PCR based on the plasmid encoding TP and a DNAP mutant (Y127A) under the control of a rhamnose inducible promoter, and we transformed the library into the strain TC1-dCas9. After overnight culture with 1 mM arabinose and 1 mM rhamnose to switch from using the genomically-encoded WT DNAP to using the plasmid-encoded DNAP mutants for replicating the o-replicon, we plated all cells on a plate with chloramphenicol to screen out all cells with chloramphenicol resistance.

We then washed off the cells from the plate and extracted the screened ODNAP library plasmids and transformed them into strain TC2-dCas9. After transformation, we did the same screening as the TC1-dCas9 screening, and got an enriched error-prone ODNAP mutant library.

### Second Round of Screening

We generated an ODNAP library by error prone PCR based on the ODNAP library obtained from the first round, and we screened this new library in the strains TC1-dCas9, TC2-dCas9, and TCB-dCas9 sequentially to gradually enrich the better error-prone ODNAP mutants.

After the final round of screening, we picked 96 colonies from the plate and sequenced the ODNAP genes. We found that mutations Y347C, D325V, K329R and N353K were highly enriched. We tested the mutation rates of the 5 most enriched ODNAP mutants by fluctuation analysis. The mutation rates were tested with 1 mM arabinose and 1 mM rhamnose (same condition as during the screening) and the copy number data were obtained with 1 mM arabinose and 5 mM rhamnose (same condition as testing the maximum copy number).

We obtained ODNAP mutants that have higher mutation rates and sufficiently high copy numbers.

### REFERENCES AND NOTES

1. M. Lynch, Evolution of the mutation rate. Trends Genet 26, 345-352 (2010).
2. M. S. Packer, D. R. Liu, Methods for the directed evolution of proteins. Nat Rev Genet 16, 379-394 (2015).
3. N. M. Low, P. H. Holliger, G. Winter, Mimicking somatic hypermutation: affinity maturation of antibodies displayed on bacteriophage using a bacterial mutator strain. J Mol Biol 260, 359-368 (1996).
4. C. M. Berman et al., An Adaptable Platform for Directed Evolution in Human Cells. J Am Chem Soc 140, 18093-18103 (2018).
5. J. G. English et al., VEGAS as a Platform for Facile Directed Evolution in Mammalian Cells. Cell 178, 748-761 e717 (2019).
6. C. E. Denes et al., The VEGAS Platform Is Unsuitable for Mammalian Directed Evolution. ACS Synth Biol 11, 3544-3549 (2022).
7. K. M. Esvelt, J. C. Carlson, D. R. Liu, A system for the continuous directed evolution of biomolecules. Nature 472, 499-503 (2011).
8. C. L. Moore, L. J. Papa, 3rd, M. D. Shoulders, A Processive Protein Chimera Introduces Mutations across Defined DNA Regions In Vivo. J Am Chem Soc 140, 11560-11564 (2018).
9. A. Cravens, O. K. Jamil, D. Kong, J. T. Sockolosky, C. D. Smolke, Polymerase-guided base editing enables in vivo mutagenesis and rapid protein engineering. Nat Commun 12, 1579 (2021).
10. X. Yi, J. Khey, R. J. Kazlauskas, M. Travisano, Plasmid hypermutation using a targeted artificial DNA replisome. Sci Adv 7, (2021).
11. S. O. Halperin et al., CRISPR-guided DNA polymerases enable diversification of all nucleotides in a tunable window. Nature 560, 248-252 (2018).
12. A. Ravikumar, G. A. Arzumanyan, M. K. A. Obadi, A. A. Javanpour, C. C. Liu, Scalable, Continuous Evolution of Genes at Mutation Rates above Genomic Error Thresholds. Cell 175, 1946-1957 e1913 (2018).
13. A. Ravikumar, A. Arrieta, C. C. Liu, An orthogonal DNA replication system in yeast. Nat Chem Biol 10, 175-177 (2014).
14. R. Tian et al., Engineered bacterial orthogonal DNA replication system for continuous evolution. Nat Chem 50 Biol, (2023).
15. M. Camps, J. Naukkarinen, B. P. Johnson, L. A. Loeb, Targeted gene evolution in Escherichia coli using a highly error-prone DNA polymerase I. Proc Natl Acad Sci USA 100, 9727-9732 (2003).
16. C. Fabret et al., Efficient gene targeted random mutagenesis in genetically stable Escherichia coli strains. Nucleic Acids Res 28, E95 (2000).
17. G. T. Hess et al., Directed evolution using dCas9-targeted somatic hypermutation in mammalian cells. Nat Methods 13, 1036-1042 (2016).
18. A. Wellner et al., Rapid generation of potent antibodies by autonomous hypermutation in yeast. Nat Chem Biol 17, 1057-1064 (2021).
19. Z. D. Blount, The unexhausted potential of E. coli. Elife 4, (2015).
20. M. M. Poranen et al., Global changes in cellular gene expression during bacteriophage PRD1 infection. J Virol 80, 8081-8088 (2006).
21. A. M. Grahn, J. K. Bamford, M. C. O'Neill, D. H. Bamford, Functional organization of the bacteriophage PRD1 genome. J Bacteriol 176, 3062-3068 (1994).
22. H. Savilahti, D. H. Bamford, Protein-primed DNA replication: role of inverted terminal repeats in the Escherichia coli bacteriophage PRD1 life cycle. J Virol 67, 4696-4703 (1993).
23. K. C. Murphy, Lambda Gam protein inhibits the helicase and chi-stimulated recombination activities of Escherichia coli RecBCD enzyme. J Bacteriol 173, 5808-5821 (1991).
24. J. Jee et al., Rates and mechanisms of bacterial mutagenesis from maximum-depth sequencing. Nature 534, 693-696 (2016).
25. H. Lee, E. Popodi, H. Tang, P. L. Foster, Rate and molecular spectrum of spontaneous mutations in the bacterium Escherichia coli as determined by whole-genome sequencing. Proc Natl Acad Sci USA 109, E2774-2783 (2012).
26. A. H. Badran, D. R. Liu, Development of potent in vivo mutagenesis plasmids with broad mutational spectra. Nat Commun 6, 8425 (2015).
27. R. Ando, H. Hama, M. Yamamoto-Hino, H. Mizuno, A. Miyawaki, An optical marker based on the UV-induced green-to-red photoconversion of a fluorescent protein. Proc Natl Acad Sci USA 99, 12651-12656 (2002).
28. R. Heim, A. B. Cubitt, R. Y. Tsien, Improved green fluorescence. Nature 373, 663-664 (1995).
29. S. H. Joshi, C. Yong, A. Gyorgy, Inducible plasmid copy number control for synthetic biology in commonly used E. coli strains. Nat Commun 13, 6691 (2022).
30. M. V. Rouches, Y. Xu, L. B. G. Cortes, G. Lambert, A plasmid system with tunable copy number. Nat Commun 13, 3908 (2022).
31. M. Barlow, B. G. Hall, Predicting evolutionary potential: in vitro evolution accurately reproduces natural evolution of the tem beta-lactamase. Genetics 160, 823-832 (2002).
32. M. Fischlechner et al., Evolution of enzyme catalysts caged in biomimetic gel-shell beads. Nat Chem 6, 791-796 (2014).
33. V. Salema, L. A. Fernandez, Escherichia coli surface display for the selection of nanobodies. Microb Biotechnol 10, 1468-1484 (2017).
34. J. K. Joung, E. I. Ramm, C. O. Pabo, A bacterial two-hybrid selection system for studying protein-DNA and protein-protein interactions. Proc Natl Acad Sci USA 97, 7382-7387 (2000).
35. G. Karimova, J. Pidoux, A. Ullmann, D. Ladant, A bacterial two-hybrid system based on a reconstituted signal transduction pathway. Proc Natl Acad Sci USA 95, 5752-5756 (1998).

### REFERENCES SPECIFICALLY FOR THE MATERIALS AND METHODS

1. W. Jiang, D. Bikard, D. Cox, F. Zhang, L. A. Marraffini, RNA-guided editing of bacterial genomes using CRISPR-Cas systems. Nat Biotechnol 31, 233-239 (2013).
2. J. F. Zurcher et al., Continuous synthesis of E. coli genome sections and Mb-scale human DNA assembly. Nature 619, 555-562 (2023).
3. P. L. Foster, Methods for determining spontaneous mutation rates. Methods Enzymol 409, 195-213 (2006).
4. B. M. Hall, C. X. Ma, P. Liang, K. K. Singh, Fluctuation analysis CalculatOR: a web tool for the determination of mutation rate using Luria-Delbruck fluctuation analysis. Bioinformatics 25, 1564-1565 (2009).
5. M. Mirdita et al., ColabFold: making protein folding accessible to all. Nat Methods 19, 679-682 (2022).
6. J. Jumper et al., Highly accurate protein structure prediction with AlphaFold. Nature 596, 583-589 (2021).
7. E. F. Pettersen et al., UCSF ChimeraX: Structure visualization for researchers, educators, and developers. Protein Sci 30, 70-82 (2021).

1. A cell comprising orthogonal DNA replication machinery, wherein the orthogonal DNA replication machinery is from or derived from a lytic phage of the Tectiviridea family or a lytic φ29-like virus of the Podoviridae family.

2. The cell of claim 1, wherein the cell is a bacterial cell or an E. coli cell.

3. The cell of claim 1 or claim 2, wherein
the cell comprises one or more non-orthogonal episomes that are capable of being replicated by the endogenous DNA polymerase, and the one or more non-orthogonal episomes encode all components of the orthogonal DNA replication machinery; and/or
at least one, a plurality, or all components of the orthogonal DNA replication machinery are encoded by the cell genome; and/or
the orthogonal DNA replication machinery is capable of replicating an orthogonal episome that does not comprise at least one, a plurality, or all components of the orthogonal DNA replication machinery.

4. The cell of any preceding claim, wherein the orthogonal DNA replication machinery replicates DNA with a lower fidelity than the cell's endogenous DNA replication machinery.

5. The cell of any preceding claim, wherein the orthogonal DNA replication machinery is from or derived from a Tectiviridae family member.

6. The cell of any preceding claim, wherein the orthogonal DNA replication machinery is from or derived from any of phage PRD1, phage PR3, phage PR4, phage PR5, phage L17, phage PR722, and phage BCE1.

7. The cell of any preceding claim, wherein the orthogonal DNA replication machinery comprises a gene encoding a Podoviridae or Tectiviridae terminal protein (TP) and a gene encoding an orthogonal DNA polymerase (ODNAP).

8. The cell of any preceding claim, wherein the orthogonal DNA replication machinery comprises a gene encoding a Podoviridae or Tectiviridae TP, a gene encoding an ODNAP, and a gene encoding a Podoviridae or Tectiviridae single-stranded DNA-binding protein (SSB).

9. The cell of any preceding claim, wherein the orthogonal DNA replication machinery comprises a gene encoding a Podoviridae or Tectiviridae double-stranded DNA-binding protein (DSB).

10. The cell of any preceding claim, wherein the orthogonal DNA replication machinery is from or derived from PRD1 bacteriophage.

11. The cell of any preceding claim, wherein the orthogonal DNA replication machinery comprises a gene encoding a PRD1 bacteriophage TP and a gene encoding an ODNAP.

12. The cell of any preceding claim, wherein the orthogonal DNA replication machinery comprises a gene encoding a PRD1 bacteriophage TP, a gene encoding an ODNAP, and a gene encoding a PRD1 bacteriophage SSB.

13. The cell of claim 12, wherein the orthogonal DNA replication machinery comprises a gene encoding a PRD1 bacteriophage DSB.

14. The cell of any one of claims 7, 8, 11, 12, and 13, wherein the ODNAP is a Podoviridae or Tectiviridae ODNAP or an engineered Podoviridae or Tectiviridae ODNAP.

15. The cell of claim 14, wherein the ODNAP is a PRD1 bacteriophage ODNAP or an engineered PRD1 bacteriophage ODNAP.

16. The cell of any preceding claim, wherein the cell comprises sequence encoding a protein that is at least 80%, 85%, 90%, 95%, 97%, 98%, 99%, or 100% similar or identical to any one, two, three, or four of SEQ ID NOs:
1, 2, 3, and 4.

17. The cell of any one of claims 7, 8, and 11 to 16, wherein the ODNAP replicates DNA with a lower fidelity than the cell's endogenous DNA polymerase.

18. The cell of claim 17, wherein the ODNAP comprises:
i) any one the following mutations or sets of mutations described with reference to SEQ ID NO: 2: T20I, N71D, N71D & L341R, H70R, D17A, D76A, D17A & D76A, D17A & D76A & L341R, and Y127A; or
ii) any one or any combination of the following mutations described with reference to SEQ ID NO: 2: D17A, T201, H70R, N71D, D76A, Y127A, Q275L, 1291V, D325V, K329R, L341R, Y347C, N353K, E364G, 1367T, Q420H, L437S, and Q446L; or
iii) any of the following sets of mutations described with reference to SEQ ID NO: 2: Y127A, Y347C, and 1367T; Y127A, 1291V, and D325V; Y127A, Y347C, N353K, E364G, and Q420H; Y127A, Q275L, K329R, Y347C, and L437S; or Y127A, Y347C, and Q446L; or
iv) any one or any combination of the following mutations described with reference to SEQ ID NO: 2: Y347C, D325V, K329R, and N353K.

19. The cell of any preceding claim, wherein at least one, a plurality, or all components of the orthogonal DNA replication machinery are encoded by a single operon.

20. The cell of any preceding claim, wherein the cell comprises a gene encoding Gam protein.

21. The cell of any preceding claim, comprising an episome that is capable of being replicated by the orthogonal DNA replication machinery.

22. The cell of claim 21, wherein the episome comprises a 5′ inverted terminal repeat (ITR) and a 3′ ITR, wherein the 5′ ITR and the 3′ ITR are compatible with the orthogonal DNA replication machinery.

23. The cell of claim 22, wherein the 5′ ITR and 3′ ITR are compatible with Tectiviridae DNA replication machinery.

24. The cell of claim 22, wherein the 5′ ITR and 3′ ITR are compatible with PRD1 bacteriophage DNA replication machinery.

25. The cell of any one of claims 21 to 24, wherein the episome comprises:
a 5′ ITR that is at least 80%, 85%, 90%, 95%, 97%, 98%, 99%, or 100% identical to SEQ ID NO: 7, or a truncation thereof, and/or a 3′ ITR that is at least 80%, 85%, 90%, 95%, 97%, 98%, 99%, or 100% identical to SEQ ID NO: 8, or a truncation thereof; or
a 5′ ITR that is at least 80%, 85%, 90%, 95%, 97%, 98%, 99%, or 100% identical to SEQ ID NO: 10 and/or a 3′ ITR that is at least 80%, 85%, 90%, 95%, 97%, 98%, 99%, or 100% identical to SEQ ID NO: 11.

26. The cell of any one of claims 21 to 25, wherein the episome is a linear plasmid.

27. The cell of any one of claims 21 to 26, wherein the episome comprises a sequence-of-interest.

28. The cell of any one of claims 1 to 27, wherein the cell comprises a circular nucleic acid molecule that comprises the sequence of an episome that is capable of being replicated by the orthogonal DNA replication machinery.

29. The cell of claim 28, wherein the episome that is capable of being replicated by the orthogonal DNA replication machinery is an episome according to any one of claims 21 to 27.

30. The cell of claim 28 or claim 29, wherein the circular nucleic acid molecule is configured so that the episome that is capable of being replicated by the orthogonal DNA replication machinery may be excised from the circular episome within the cell.

31. A linear plasmid comprising a 5′ ITR and a 3′ ITR, each of which is compatible with DNA replication machinery from or derived from a lytic phage of the Tectiviridea family or a lytic φ29-like virus of the Podoviridae family.

32. The linear plasmid of claim 31, wherein the 5′ ITR and 3′ ITR are compatible with PRD1 bacteriophage DNA replication machinery.

33. The linear plasmid of claim 31 or claim 32, wherein the linear plasmid comprises:
a 5′ ITR that is at least 80%, 85%, 90%, 95%, 97%, 98%, 99%, or 100% identical to SEQ ID NO: 7, or a truncation thereof and/or a 3′ ITR that is at least 80%, 85%, 90%, 95%, 97%, 98%, 99%, or 100% identical to SEQ ID NO: 8, or a truncation thereof; or
a 5′ ITR that is at least 80%, 85%, 90%, 95%, 97%, 98%, 99%, or 100% identical to SEQ ID NO: 10 and/or a 3′ ITR that is at least 80%, 85%, 90%, 95%, 97%, 98%, 99%, or 100% identical to SEQ ID NO: 11.

34. The linear plasmid of any one of claims 31 to 33, wherein the plasmid does not encode one, two, three, or four of a TP, a DNA polymerase, an SSB, and a DSB.

35. The linear plasmid of any one of claims 31 to 34, wherein the plasmid comprises a sequence-of-interest.

36. The linear plasmid of claim 35, wherein the sequence-of-interest is not a sequence endogenous to Podoviridae or Tectiviridae family bacteriophages.

37. A circular nucleic acid molecule comprising the sequence of a linear plasmid according of any one of claims 31 to 36.

38. The circular nucleic acid molecule of claim 37, wherein the circular nucleic acid molecule is a circular episome, a circular plasmid, or a bacterial artificial chromosome.

39. The circular nucleic acid molecule of claim 37 of claim 38, wherein the circular nucleic acid molecule comprises one or more sites positioned such that the linear plasmid may be excised from the circular nucleic acid molecule.

40. A method of maintaining a linear plasmid, wherein the method comprises:
i) providing a cell according to any one of claims 21 to 30, transferring a linear plasmid according to any one of claims 31 to 36 to a cell according to any one of claims 1 to 20, or excising a linear plasmid from a circular nucleic acid molecule according to any one of claims 37 to 39 within a cell according to any one of claims 1 to 20; and
ii) incubating the cell under conditions conducive to growth.

41. Use of a cell according to any one of claims 1 to 30, a linear plasmid according to any one of claims 31 to 36, or a circular nucleic acid molecule according to any one of claims 37 to 39 for evolving a sequence-of-interest.

42. A method of evolving a sequence-of-interest, wherein the method comprises:
i) providing a cell according to any one of claims 21 to 30, transferring a linear plasmid according to any one of claims 31 to 36 to a cell according to any one of claims 1 to 20, or excising a linear plasmid from a circular nucleic acid molecule according to any one of claims 37 to 39 within a cell according to any one of claims 1 to 20, wherein the episome or linear plasmid comprises a sequence-of-interest and wherein the orthogonal DNA replication machinery replicates DNA with a lower fidelity than the cell's endogenous DNA replication machinery;
ii) incubating the cell under conditions conducive to growth; and
iii) exposing the cell to a selection condition.

43. The method of claim 42, wherein the method comprises:
iv) identifying the sequence of a sequence-of-interest from a cell meeting the selection condition.

44. The method of claim 43, wherein the method comprises:
v) making a polypeptide or nucleic acid encoded by the identified sequence-of-interest.

45. A method of making a polypeptide or nucleic acid, wherein the method comprises:
i) providing a sequence-of-interest identified by the method of any one of claims 42 to 44, and
ii) producing a polypeptide or nucleic acid according to said sequence.
